SEQUENCE LISTING (201 sequences)

SEQ ID NO: 1 - protein, 6 aa, Artificial
  Synthetic construct
  DISULFID (1)..(6)
  MOD_RES (1)..(1): ACETYLATION
  MOD_RES (3)..(3): D form
  MOD_RES (6)..(6): AMIDATION
CHFRWC

SEQ ID NO: 2 - protein, 9 aa, Artificial
  Synthetic construct
  MISC_FEATURE (1)..(1): Xaa = Cysteic acid
  MOD_RES (1)..(1): ACETYLATION
  DISULFID (3)..(9)
  MOD_RES (6)..(6): D form
  MOD_RES (9)..(9): AMIDATION
XRCAHFRWC

SEQ ID NO: 3 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCAHFRWC

SEQ ID NO: 4 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCRHFRWC

SEQ ID NO: 5 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCNHFRWC

SEQ ID NO: 6 - protein, 7 aa, Artificial
  Synthetic construct
  MOD_RES (1)..(1): ACETYLATION
  DISULFID (1)..(7)
  MOD_RES (4)..(4): D form
  MOD_RES (7)..(7): AMIDATION
CDHFRWC

SEQ ID NO: 7 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCDHFRWC

SEQ ID NO: 8 - protein, 7 aa, Artificial
  Synthetic construct
  MOD_RES (1)..(1): ACETYLATION
  DISULFID (1)..(7)
  MOD_RES (4)..(4): D form
  MOD_RES (7)..(7): AMIDATION
CQHFRWC

SEQ ID NO: 9 - protein, 9 aa, Artificial
  Synthetic construct
  MOD_RES (1)..(1): ACETYLATION
  DISULFID (3)..(9)
  MOD_RES (6)..(6): D form
YRCQHFRWC

SEQ ID NO: 10 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): Methoxy substituted for OH
YRCQHFRWC

SEQ ID NO: 11 - protein, 9 aa, Artificial
  Synthetic construct
  DISULFID (3)..(9)
  MOD_RES (6)..(6): D form
  MOD_RES (9)..(9): AMIDATION
YRCGHFRWC

SEQ ID NO: 12 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCGHFRWC

SEQ ID NO: 13 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCHHFRWC

SEQ ID NO: 14 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCIHFRWC

SEQ ID NO: 15 - protein, 7 aa, Artificial
  Synthetic construct
  MOD_RES (1)..(1): ACETYLATION
  DISULFID (1)..(7)
  MOD_RES (4)..(4): D form
  MOD_RES (7)..(7): AMIDATION
CLHFRWC

SEQ ID NO: 16 - protein, 7 aa, Artificial
  Synthetic construct
  MOD_RES (1)..(1): ACETYLATION
  DISULFID (1)..(7)
  MOD_RES (4)..(4): D form
  MOD_RES (7)..(7): AMIDATION
CKHFRWC

SEQ ID NO: 17 - protein, 9 aa, Artificial
  Synthetic construct
  MOD_RES (1)..(1): METHYLATION
  DISULFID (3)..(9)
  MOD_RES (6)..(6): D form
  MOD_RES (9)..(9): AMIDATION
YRCMHFRWC

SEQ ID NO: 18 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCMHFRWC

SEQ ID NO: 19 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCFHFRWC

SEQ ID NO: 20 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCPHFRWC

SEQ ID NO: 21 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCSHFRWC

SEQ ID NO: 22 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCTHFRWC

SEQ ID NO: 23 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCWHFRWC

SEQ ID NO: 24 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCYHFRWC

SEQ ID NO: 25 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCVHFRWC

SEQ ID NO: 26 - protein, 8 aa, Artificial
  Synthetic construct
  MOD_RES (1)..(1): ACETYLATION
  DISULFID (2)..(8)
  MISC_FEATURE (3)..(3): Xaa = cysteic acid
  MOD_RES (5)..(5): D form
  MOD_RES (8)..(8): AMIDATION
RCXHFRWC

SEQ ID NO: 27 - protein, 8 aa, Artificial
  Synthetic construct
  MOD_RES (1)..(1): ACETYLATION
  MOD_RES (1)..(1): D form
  DISULFID (2)..(8)
  MISC_FEATURE (3)..(3): Xaa = cysteic acid
  MOD_RES (5)..(5): D form
  MOD_RES (8)..(8): AMIDATION
RCXHFRWC

SEQ ID NO: 28 - protein, 9 aa, Artificial
  Synthetic construct
  MOD_RES (1)..(1): ACETYLATION
  DISULFID (3)..(9)
  MISC_FEATURE (4)..(4): Xaa = cysteic acid
  MOD_RES (6)..(6): D form
  MOD_RES (9)..(9): AMIDATION
YRCXHFRWC

SEQ ID NO: 29 - protein, 7 aa, Artificial
  Synthetic construct
  DISULFID (1)..(7)
  MOD_RES (4)..(4): D form
  MOD_RES (7)..(7): AMIDATION
CEHFRWC

SEQ ID NO: 30 - protein, 7 aa, Artificial
  Synthetic construct
  MOD_RES (1)..(1): ACETYLATION
  DISULFID (1)..(7)
  MOD_RES (4)..(4): D form
  MOD_RES (7)..(7): AMIDATION
CEHFRWC

SEQ ID NO: 31 - protein, 7 aa, Artificial
  Synthetic construct
  MOD_RES (1)..(1): ACETYLATION
  DISULFID (1)..(7)
  MOD_RES (4)..(4): 4-fluoro substituted, D form
  MOD_RES (7)..(7): AMIDATION
CEHFRWC

SEQ ID NO: 32 - protein, 7 aa, Artificial
  Synthetic construct
  MOD_RES (1)..(1): ACETYLATION
  DISULFID (1)..(7)
  MOD_RES (4)..(4): 4-chloro substituted, D form
  MOD_RES (7)..(7): AMIDATION
CEHFRWC

SEQ ID NO: 33 - protein, 7 aa, Artificial
  Synthetic construct
  MOD_RES (1)..(1): ACETYLATION
  DISULFID (1)..(7)
  MOD_RES (4)..(4): 4-bromo substituted, D form
  MOD_RES (7)..(7): AMIDATION
CEHFRWC

SEQ ID NO: 34 - protein, 7 aa, Artificial
  Synthetic construct
  MOD_RES (1)..(1): ACETYLATION
  DISULFID (1)..(7)
  MOD_RES (3)..(3): 1-methyl substituted
  MOD_RES (4)..(4): D form
  MOD_RES (7)..(7): AMIDATION
CEHFRWC

SEQ ID NO: 35 - protein, 9 aa, Artificial
  Synthetic construct
  MOD_RES (1)..(1): ACETYLATION
  DISULFID (1)..(7)
  MOD_RES (4)..(4): D form
  MOD_RES (9)..(9): AMIDATION
CEHFRWCKP

SEQ ID NO: 36 - protein, 9 aa, Artificial
  Synthetic construct
  MOD_RES (1)..(1): ACETYLATION
  DISULFID (1)..(7)
  MOD_RES (4)..(4): D form
  MOD_RES (9)..(9): AMIDATION
CEHFRWCSP

SEQ ID NO: 37 - protein, 7 aa, Artificial
  Synthetic construct
  MOD_RES (1)..(1): N-propionyl substituted
  DISULFID (1)..(7)
  MOD_RES (4)..(4): D form
  MOD_RES (7)..(7): AMIDATION
CEHFRWC

SEQ ID NO: 38 - protein, 7 aa, Artificial
  Synthetic construct
  MOD_RES (1)..(1): N-butyryl substituted
  DISULFID (1)..(7)
  MOD_RES (4)..(4): D form
  MOD_RES (7)..(7): AMIDATION
CEHFRWC

SEQ ID NO: 39 - protein, 7 aa, Artificial
  Synthetic construct
  MOD_RES (1)..(1): N-valeryl substituted
  DISULFID (1)..(7)
  MOD_RES (4)..(4): D form
  MOD_RES (7)..(7): AMIDATION
CEHFRWC

SEQ ID NO: 40 - protein, 7 aa, Artificial
  Synthetic construct
  MOD_RES (1)..(1): 3-guanidinopropionyl substituted
  DISULFID (1)..(7)
  MOD_RES (4)..(4): D form
  MOD_RES (7)..(7): AMIDATION
CEHFRWC

SEQ ID NO: 41 - protein, 7 aa, Artificial
  Synthetic construct
  MOD_RES (1)..(1): 4-guanidinobutyryl substituted
  DISULFID (1)..(7)
  MOD_RES (4)..(4): D form
  MOD_RES (7)..(7): AMIDATION
CEHFRWC

SEQ ID NO: 42 - protein, 7 aa, Artificial
  Synthetic construct
  MOD_RES (1)..(1): 5-guanidinovaleryl substituted
  DISULFID (1)..(7)
  MOD_RES (4)..(4): D form
  MOD_RES (7)..(7): AMIDATION
CEHFRWC

SEQ ID NO: 43 - protein, 7 aa, Artificial
  Synthetic construct
  MOD_RES (1)..(1): acetyl-diaminopropionyl substituted
  DISULFID (1)..(7)
  MOD_RES (4)..(4): D form
  MOD_RES (7)..(7): AMIDATION
CEHFRWC

SEQ ID NO: 44 - protein, 7 aa, Artificial
  Synthetic construct
  MOD_RES (1)..(1): acetyl-diaminobutyryl substituted
  DISULFID (1)..(7)
  MOD_RES (4)..(4): D form
  MOD_RES (7)..(7): AMIDATION
CEHFRWC

SEQ ID NO: 45 - protein, 8 aa, Artificial
  Synthetic construct
  DISULFID (2)..(8)
  MOD_RES (5)..(5): D form
RCEHFRWC

SEQ ID NO: 46 - protein, 8 aa, Artificial
  Synthetic construct
  MOD_RES (1)..(1): D form
  DISULFID (2)..(8)
  MOD_RES (5)..(5): D form
  MOD_RES (8)..(8): AMIDATION
RCEHFRWC

SEQ ID NO: 47 - protein, 8 aa, Artificial
  Synthetic construct
  MOD_RES (1)..(1): ACETYLATION
  MOD_RES (1)..(1): D form
  DISULFID (2)..(8)
  MOD_RES (8)..(8): AMIDATION
RCEHFRWC

SEQ ID NO: 48 - protein, 8 aa, Artificial
  Synthetic construct
  MOD_RES (1)..(1): ACETYLATION
  DISULFID (2)..(8)
  MOD_RES (5)..(5): D form
  MOD_RES (8)..(8): AMIDATION
RCEHFRWC

SEQ ID NO: 49 - protein, 8 aa, Artificial
  Synthetic construct
  MOD_RES (1)..(1): ACETYLATION
  DISULFID (2)..(8)
  MOD_RES (5)..(5): D form
RCEHFRWC

SEQ ID NO: 50 - protein, 8 aa, Artificial
  Synthetic construct
  MOD_RES (1)..(1): ACETYLATION
  DISULFID (2)..(8)
  MOD_RES (5)..(5): 4-chloro substituted, D form
  MOD_RES (8)..(8): AMIDATION
RCEHFRWC

SEQ ID NO: 51 - protein, 8 aa, Artificial
  Synthetic construct
  MOD_RES (1)..(1): ACETYLATION
  DISULFID (2)..(8)
  MOD_RES (4)..(4): 1-methyl substituted
  MOD_RES (5)..(5): D form
  MOD_RES (8)..(8): AMIDATION
RCEHFRWC

SEQ ID NO: 52 - protein, 8 aa, Artificial
  Synthetic construct
  MOD_RES (1)..(1): ACETYLATION
  MOD_RES (1)..(1): D form
  DISULFID (2)..(8)
  MOD_RES (5)..(5): D form
  MOD_RES (8)..(8): AMIDATION
RCEHFRWC

SEQ ID NO: 53 - protein, 8 aa, Artificial
  Synthetic construct
  MOD_RES (1)..(1): ACETYLATION
  MOD_RES (1)..(1): D form
  DISULFID (2)..(8)
  MOD_RES (5)..(5): D form
RCEHFRWC

SEQ ID NO: 54 - protein, 8 aa, Artificial
  Synthetic construct
  MOD_RES (1)..(1): ACETYLATION
  MISC_FEATURE (1)..(1): Xaa = homoarginine
  DISULFID (2)..(8)
  MOD_RES (5)..(5): D form
  MOD_RES (8)..(8): AMIDATION
XCEHFRWC

SEQ ID NO: 55 - protein, 8 aa, Artificial
  Synthetic construct
  MOD_RES (1)..(1): ACETYLATION
  MISC_FEATURE (1)..(1): Xaa = citrulline
  DISULFID (2)..(8)
  MOD_RES (5)..(5): D form
  MOD_RES (8)..(8): AMIDATION
XCEHFRWC

SEQ ID NO: 56 - protein, 8 aa, Artificial
  Synthetic construct
  MOD_RES (1)..(1): ACETYLATION
  MISC_FEATURE (1)..(1): Xaa = citrulline
  DISULFID (2)..(8)
  MOD_RES (4)..(4): 1-methyl substituted
  MOD_RES (5)..(5): D form
  MOD_RES (8)..(8): AMIDATION
XCEHFRWC

SEQ ID NO: 57 - protein, 8 aa, Artificial
  Synthetic construct
  MOD_RES (1)..(1): ACETYLATION
  DISULFID (2)..(8)
  MOD_RES (5)..(5): D form
  MOD_RES (8)..(8): AMIDATION
LCEHFRWC

SEQ ID NO: 58 - protein, 8 aa, Artificial
  Synthetic construct
  MOD_RES (1)..(1): ACETYLATION
  DISULFID (2)..(8)
  MOD_RES (5)..(5): D form
  MOD_RES (8)..(8): AMIDATION
KCEHFRWC

SEQ ID NO: 59 - protein, 8 aa, Artificial
  Synthetic construct
  MOD_RES (1)..(1): ACETYLATION
  MISC_FEATURE (1)..(1): Xaa = N(epsilon)-isopropyl lysine
  DISULFID (2)..(8)
  MOD_RES (5)..(5): D form
  MOD_RES (8)..(8): AMIDATION
XCEHFRWC

SEQ ID NO: 60 - protein, 8 aa, Artificial
  Synthetic construct
  MOD_RES (1)..(1): ACETYLATION
  MISC_FEATURE (1)..(1): Xaa = norleucine
  DISULFID (2)..(8)
  MOD_RES (5)..(5): D form
  MOD_RES (8)..(8): AMIDATION
XCEHFRWC

SEQ ID NO: 61 - protein, 10 aa, Artificial
  Synthetic construct
  MOD_RES (1)..(1): ACETYLATION
  MISC_FEATURE (1)..(1): Xaa = norleucine
  DISULFID (2)..(8)
  MOD_RES (5)..(5): D form
  MOD_RES (10)..(10): AMIDATION
XCEHFRWCSP

SEQ ID NO: 62 - protein, 8 aa, Artificial
  Synthetic construct
  MOD_RES (1)..(1): ACETYLATION
  MISC_FEATURE (1)..(1): Xaa = Ornithine
  DISULFID (2)..(8)
  MOD_RES (5)..(5): D form
  MOD_RES (8)..(8): AMIDATION
XCEHFRWC

SEQ ID NO: 63 - protein, 8 aa, Artificial
  Synthetic construct
  MOD_RES (1)..(1): ACETYLATION
  DISULFID (2)..(8)
  MOD_RES (5)..(5): D form
  MOD_RES (8)..(8): AMIDATION
VCEHFRWC

SEQ ID NO: 64 - protein, 8 aa, Artificial
  Synthetic construct
  MOD_RES (1)..(1): N-(2-naphthalenesulfonyl) substituted, D form
  DISULFID (2)..(8)
  MOD_RES (5)..(5): D form
  MOD_RES (8)..(8): AMIDATION
RCEHFRWC

SEQ ID NO: 65 - protein, 8 aa, Artificial
  Synthetic construct
  MOD_RES (1)..(1): N-(2-naphthalenesulfonylamino-4-oxo-butyryl) substituted
  MOD_RES (1)..(1): D form
  DISULFID (2)..(8)
  MOD_RES (5)..(5): D form
  MOD_RES (8)..(8): AMIDATION
RCEHFRWC

SEQ ID NO: 66 - protein, 8 aa, Artificial
  Synthetic construct
  MOD_RES (1)..(1): 3-(4-hydroxyphenyl)propionyl substituted
  DISULFID (2)..(8)
  MOD_RES (5)..(5): D form
  MOD_RES (8)..(8): AMIDATION
RCEHFRWC

SEQ ID NO: 67 - protein, 8 aa, Artificial
  Synthetic construct
  MOD_RES (1)..(1): 3-(4-methylbenzoyl)propionyl) substituted
  DISULFID (2)..(8)
  MOD_RES (5)..(5): D form
  MOD_RES (8)..(8): AMIDATION
RCEHFRWC

SEQ ID NO: 68 - protein, 9 aa, Artificial
  Synthetic construct
  DISULFID (3)..(9)
  MOD_RES (6)..(6): D form
  MOD_RES (9)..(9): AMIDATION
YRCEHFRWC

SEQ ID NO: 69 - protein, 9 aa, Artificial
  Synthetic construct
  DISULFID (3)..(9)
  MOD_RES (6)..(6): D form
YRCEHFRWC

SEQ ID NO: 70 - protein, 9 aa, Artificial
  Synthetic construct
  DISULFID (3)..(9)
  MOD_RES (6)..(6): D form
  MOD_RES (9)..(9): NH-(CH2)6-NH2 substituted
YRCEHFRWC

SEQ ID NO: 71 - protein, 10 aa, Artificial
  Synthetic construct
  DISULFID (3)..(9)
  MOD_RES (6)..(6): D form
  MOD_RES (10)..(10): AMIDATION
YRCEHFRWCE

SEQ ID NO: 72 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCEHFRWC

SEQ ID NO: 73 - protein, 9 aa, Artificial
  Synthetic construct
  MOD_RES (1)..(1): ACETYLATION
  DISULFID (3)..(9)
  MOD_RES (6)..(6): D form
YRCEHFRWC

SEQ ID NO: 74 - protein, 9 aa, Artificial
  Synthetic construct
  MOD_RES (1)..(1): N-succinyl substituted
  DISULFID (3)..(9)
  MOD_RES (6)..(6): D form
  MOD_RES (9)..(9): AMIDATION
YRCEHFRWC

SEQ ID NO: 75 - protein, 9 aa, Artificial
  Synthetic construct
  MOD_RES (1)..(1): N-glutaryl substituted
  DISULFID (3)..(9)
  MOD_RES (6)..(6): D form
  MOD_RES (9)..(9): AMIDATION
YRCEHFRWC

SEQ ID NO: 76 - protein, 9 aa, Artificial
  Synthetic construct
  MOD_RES (1)..(1): N-glutaryl substituted
  DISULFID (3)..(9)
  MOD_RES (6)..(6): D form
YRCEHFRWC

SEQ ID NO: 77 - protein, 9 aa, Artificial
  Synthetic construct
  MOD_RES (1)..(1): gluconoyl substituted
  DISULFID (3)..(9)
  MOD_RES (6)..(6): D form
  MOD_RES (9)..(9): AMIDATION
YRCEHFRWC

SEQ ID NO: 78 - protein, 9 aa, Artificial
  Synthetic construct
  MOD_RES (1)..(1): ACETYLATION
  DISULFID (3)..(9)
  MOD_RES (6)..(6): D form
  MISC_FEATURE (9)..(9): Xaa = Cys reduced from amino acid to amino alcohol
YRCEHFRWX

SEQ ID NO: 79 - protein, 9 aa, Artificial
  Synthetic construct
  MOD_RES (1)..(1): ACETYLATION
  MOD_RES (2)..(2): D form
  DISULFID (3)..(9)
  MOD_RES (6)..(6): D form
  MOD_RES (9)..(9): AMIDATION
YRCEHFRWC

SEQ ID NO: 80 - protein, 9 aa, Artificial
  Synthetic construct
  MOD_RES (1)..(1): ACETYLATION
  DISULFID (3)..(9)
  MOD_RES (3)..(3): D form
  MOD_RES (6)..(6): D form
  MOD_RES (9)..(9): AMIDATION
YRCEHFRWC

SEQ ID NO: 81 - protein, 9 aa, Artificial
  Synthetic construct
  MOD_RES (1)..(1): ACETYLATION
  DISULFID (3)..(9)
  MOD_RES (5)..(5): 1-methyl substituted
  MOD_RES (6)..(6): D form
  MOD_RES (9)..(9): AMIDATION
YRCEHFRWC

SEQ ID NO: 82 - protein, 9 aa, Artificial
  Synthetic construct
  MOD_RES (1)..(1): ACETYLATION
  DISULFID (3)..(9)
  MOD_RES (5)..(5): 1-methyl substituted, D form
  MOD_RES (6)..(6): D form
  MOD_RES (9)..(9): AMIDATION
YRCEHFRWC

SEQ ID NO: 83 - protein, 9 aa, Artificial
  Synthetic construct
  MOD_RES (1)..(1): ACETYLATION
  DISULFID (3)..(9)
  MOD_RES (6)..(6): 4-fluoro substituted, D form
  MOD_RES (9)..(9): AMIDATION
YRCEHFRWC

SEQ ID NO: 84 - protein, 9 aa, Artificial
  Synthetic construct
  MOD_RES (1)..(1): ACETYLATION
  DISULFID (3)..(9)
  MOD_RES (5)..(5): 1-methyl substituted
  MOD_RES (6)..(6): 4-fluoro substituted, D form
  MOD_RES (9)..(9): AMIDATION
YRCEHFRWC

SEQ ID NO: 85 - protein, 9 aa, Artificial
  Synthetic construct
  MOD_RES (1)..(1): ACETYLATION
  DISULFID (3)..(9)
  MOD_RES (5)..(5): 1-methyl substituted, D form
  MOD_RES (6)..(6): 4-fluoro substituted, D form
  MOD_RES (9)..(9): AMIDATION
YRCEHFRWC

SEQ ID NO: 86 - protein, 9 aa, Artificial
  Synthetic construct
  MOD_RES (1)..(1): ACETYLATION
  DISULFID (3)..(9)
  MOD_RES (6)..(6): 4-chloro substituted, D form
  MOD_RES (9)..(9): AMIDATION
YRCEHFRWC

SEQ ID NO: 87 - protein, 8 aa, Artificial
  Synthetic construct
  MOD_RES (1)..(1): ACETYLATION
  DISULFID (2)..(8)
  MOD_RES (4)..(4): 1-methyl substituted
  MOD_RES (5)..(5): 4-chloro substituted, D form
  MOD_RES (8)..(8): AMIDATION
RCEHFRWC

SEQ ID NO: 88 - protein, 9 aa, Artificial
  Synthetic construct
  MOD_RES (1)..(1): ACETYLATION
  DISULFID (3)..(9)
  MOD_RES (5)..(5): 1-methyl substituted, D form
  MOD_RES (6)..(6): 4-chloro substituted, D form
  MOD_RES (9)..(9): AMIDATION
YRCEHFRWC

SEQ ID NO: 89 - protein, 9 aa, Artificial
  Synthetic construct
  MOD_RES (1)..(1): ACETYLATION
  DISULFID (3)..(9)
  MOD_RES (6)..(6): 4-bromo substituted, D form
  MOD_RES (9)..(9): AMIDATION
YRCEHFRWC

SEQ ID NO: 90 - protein, 9 aa, Artificial
  Synthetic construct
  MOD_RES (1)..(1): ACETYLATION
  DISULFID (3)..(9)
  MOD_RES (5)..(5): 1-methyl substituted
  MOD_RES (6)..(6): 4-bromo substituted, D form
  MOD_RES (9)..(9): AMIDATION
YRCEHFRWC

SEQ ID NO: 91 - protein, 9 aa, Artificial
  Synthetic construct
  MOD_RES (1)..(1): ACETYLATION
  DISULFID (3)..(9)
  MOD_RES (5)..(5): 1-methyl substituted, D form
  MOD_RES (6)..(6): 4-bromo substituted, D form
  MOD_RES (9)..(9): AMIDATION
YRCEHFRWC

SEQ ID NO: 92 - protein, 9 aa, Artificial
  Synthetic construct
  MOD_RES (1)..(1): ACETYLATION
  DISULFID (3)..(9)
  MOD_RES (6)..(6): 4-methyl substituted, D form
  MOD_RES (9)..(9): AMIDATION
YRCEHFRWC

SEQ ID NO: 93 - protein, 9 aa, Artificial
  Synthetic construct
  MOD_RES (1)..(1): ACETYLATION
  DISULFID (3)..(9)
  MOD_RES (6)..(6): 4-methoxy substituted, D form
  MOD_RES (9)..(9): AMIDATION
YRCEHFRWC

SEQ ID NO: 94 - protein, 9 aa, Artificial
  Synthetic construct
  MOD_RES (1)..(1): ACETYLATION
  DISULFID (3)..(9)
  MOD_RES (5)..(5): 1-methyl substituted
  MOD_RES (6)..(6): 4-methoxy substituted, D form
  MOD_RES (9)..(9): AMIDATION
YRCEHFRWC

SEQ ID NO: 95 - protein, 9 aa, Artificial
  Synthetic construct
  MOD_RES (1)..(1): ACETYLATION
  DISULFID (3)..(9)
  MOD_RES (5)..(5): 1-methyl substituted, D form
  MOD_RES (6)..(6): 4-methoxy substituted, D form
  MOD_RES (9)..(9): AMIDATION
YRCEHFRWC

SEQ ID NO: 96 - protein, 9 aa, Artificial
  Synthetic construct
  MOD_RES (1)..(1): ACETYLATION
  DISULFID (3)..(9)
  MOD_RES (5)..(5): 3-methyl substituted
  MOD_RES (6)..(6): D form
  MOD_RES (9)..(9): AMIDATION
YRCEHFRWC

SEQ ID NO: 97 - protein, 9 aa, Artificial
  Synthetic construct
  MOD_RES (1)..(1): ACETYLATION
  DISULFID (3)..(9)
  MOD_RES (5)..(5): 5-methyl substituted
  MOD_RES (6)..(6): D form
  MOD_RES (9)..(9): AMIDATION
YRCEHFRWC

SEQ ID NO: 98 - protein, 9 aa, Artificial
  Synthetic construct
  MOD_RES (1)..(1): ACETYLATION
  DISULFID (3)..(9)
  MOD_RES (5)..(5): 5-methyl substituted, D form
  MOD_RES (6)..(6): D form
  MOD_RES (9)..(9): AMIDATION
YRCEHFRWC

SEQ ID NO: 99 - protein, 9 aa, Artificial
  Synthetic construct
  MOD_RES (1)..(1): ACETYLATION
  DISULFID (3)..(9)
  MOD_RES (5)..(5): 1-benzyl substituted
  MOD_RES (6)..(6): D form
  MOD_RES (9)..(9): AMIDATION
YRCEHFRWC

SEQ ID NO: 100 - protein, 9 aa, Artificial
  Synthetic construct
  MOD_RES (1)..(1): ACETYLATION
  DISULFID (3)..(9)
  MOD_RES (5)..(5): 1-benzyl substituted, D form
  MOD_RES (6)..(6): D form
  MOD_RES (9)..(9): AMIDATION
YRCEHFRWC

SEQ ID NO: 101 - protein, 9 aa, Artificial
  Synthetic construct
  MOD_RES (1)..(1): ACETYLATION
  DISULFID (3)..(9)
  MOD_RES (5)..(5): 1-benzyloxymethyl substituted
  MOD_RES (6)..(6): D form
  MOD_RES (9)..(9): AMIDATION
YRCEHFRWC

SEQ ID NO: 102 - protein, 9 aa, Artificial
  Synthetic construct
  MOD_RES (1)..(1): ACETYLATION
  DISULFID (3)..(9)
  MOD_RES (5)..(5): 1-pyrazolyl substituted
  MOD_RES (6)..(6): D form
  MOD_RES (9)..(9): AMIDATION
YRCEAFRWC

SEQ ID NO: 103 - protein, 9 aa, Artificial
  Synthetic construct
  MOD_RES (1)..(1): ACETYLATION
  DISULFID (3)..(9)
  MOD_RES (5)..(5): 4-phenyl-1H-imidazol-2-yl substituted
  MOD_RES (6)..(6): D form
  MOD_RES (9)..(9): AMIDATION
YRCEAFRWC

SEQ ID NO: 104 - protein, 9 aa, Artificial
  Synthetic construct
  MOD_RES (1)..(1): ACETYLATION
  DISULFID (3)..(9)
  MOD_RES (5)..(5): 4-phenyl-1H-imidazol-2-yl substituted, D form
  MOD_RES (6)..(6): D form
  MOD_RES (9)..(9): AMIDATION
YRCEAFRWC

SEQ ID NO: 105 - protein, 9 aa, Artificial
  Synthetic construct
  MOD_RES (1)..(1): ACETYLATION
  DISULFID (3)..(9)
  MOD_RES (5)..(5): 2-pyrazine substituted
  MOD_RES (6)..(6): D form
  MOD_RES (9)..(9): AMIDATION
YRCEAFRWC

SEQ ID NO: 106 - protein, 9 aa, Artificial
  Synthetic construct
  MOD_RES (1)..(1): ACETYLATION
  DISULFID (3)..(9)
  MOD_RES (5)..(5): beta-(1,2,4-triazol-3-yl) substituted
  MOD_RES (6)..(6): D form
  MOD_RES (9)..(9): AMIDATION
YRCEAFRWC

SEQ ID NO: 107 - protein, 9 aa, Artificial
  Synthetic construct
  MOD_RES (1)..(1): ACETYLATION
  DISULFID (3)..(9)
  MOD_RES (5)..(5): beta-(1,2,4-triazol-3-yl) substituted, D form
  MOD_RES (6)..(6): D form
  MOD_RES (9)..(9): AMIDATION
YRCEAFRWC

SEQ ID NO: 108 - protein, 9 aa, Artificial
  Synthetic construct
  MOD_RES (1)..(1): ACETYLATION
  DISULFID (3)..(9)
  MOD_RES (5)..(5): beta-((1-benzyl)-1,2,4-triazol-3-yl) substituted
  MOD_RES (6)..(6): D form
  MOD_RES (9)..(9): AMIDATION
YRCEAFRWC

SEQ ID NO: 109 - protein, 9 aa, Artificial
  Synthetic construct
  MOD_RES (1)..(1): ACETYLATION
  DISULFID (3)..(9)
  MOD_RES (5)..(5): beta-((1-benzyl)-1,2,4-triazol-3-yl) substituted, D form
  MOD_RES (6)..(6): D form
  MOD_RES (9)..(9): AMIDATION
YRCEAFRWC

SEQ ID NO: 110 - protein, 9 aa, Artificial
  Synthetic construct
  MOD_RES (1)..(1): ACETYLATION
  DISULFID (3)..(9)
  MOD_RES (5)..(5): beta-(2-furyl) substituted
  MOD_RES (6)..(6): D form
  MOD_RES (9)..(9): AMIDATION
YRCEAFRWC

SEQ ID NO: 111 - protein, 9 aa, Artificial
  Synthetic construct
  MOD_RES (1)..(1): ACETYLATION
  DISULFID (3)..(9)
  MOD_RES (5)..(5): beta-(thien-2-yl) substituted
  MOD_RES (6)..(6): D form
  MOD_RES (9)..(9): AMIDATION
YRCEAFRWC

SEQ ID NO: 112 - protein, 9 aa, Artificial
  Synthetic construct
  MOD_RES (1)..(1): ACETYLATION
  DISULFID (3)..(9)
  MOD_RES (5)..(5): beta-(1,3-thiazol-4-yl) substituted
  MOD_RES (6)..(6): D form
  MOD_RES (9)..(9): AMIDATION
YRCEAFRWC

SEQ ID NO: 113 - protein, 9 aa, Artificial
  Synthetic construct
  MOD_RES (1)..(1): ACETYLATION
  DISULFID (3)..(9)
  MOD_RES (5)..(5): beta-(pyridin-4-yl) substituted
  MOD_RES (6)..(6): D form
  MOD_RES (9)..(9): AMIDATION
YRCEAFRWC

SEQ ID NO: 114 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): glycinol substituted
YRCEHFRWC

SEQ ID NO: 115 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): 2-(2-aminoethoxy)ethanol substituted
YRCEHFRWC

SEQ ID NO: 116 - protein, 10 aa, Artificial
  Synthetic construct
  MOD_RES (1)..(1): ACETYLATION
  DISULFID (3)..(9)
  MOD_RES (6)..(6): D form
  MISC_FEATURE (10)..(10): Xaa = Ser reduced from amino acid to amino alcohol
YRCEHFRWCX

SEQ ID NO: 117 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): NH-(CH2)6-NH2 substituted
YRCEHFRWC

SEQ ID NO: 118 - protein, 10 aa, Artificial
  Synthetic construct
  MOD_RES (1)..(1): ACETYLATION
  DISULFID (3)..(9)
  MOD_RES (6)..(6): D form
  MOD_RES (10)..(10): AMIDATION
YRCEHFRWCE

SEQ ID NO: 119 - protein, 11 aa, Artificial
  Synthetic construct
  MOD_RES (1)..(1): ACETYLATION
  DISULFID (3)..(9)
  MOD_RES (6)..(6): D form
  MOD_RES (11)..(11): AMIDATION
YRCEHFRWCSP

SEQ ID NO: 120 - protein, 11 aa, Artificial
  Synthetic construct
  MOD_RES (1)..(1): ACETYLATION
  DISULFID (3)..(9)
  MOD_RES (6)..(6): D form
  MISC_FEATURE (11)..(11): Xaa = Pro reduced from amino acid to amino alcohol
YRCEHFRWCSX

SEQ ID NO: 121 - protein, 11 aa, Artificial
  Synthetic construct
  MOD_RES (1)..(1): ACETYLATION
  DISULFID (3)..(9)
  MOD_RES (6)..(6): D form
  MOD_RES (11)..(11): AMIDATION
YRCEHFRWCKP

SEQ ID NO: 122 - protein, 11 aa, Artificial
  Synthetic construct
  MOD_RES (1)..(1): ACETYLATION
  DISULFID (3)..(9)
  MOD_RES (6)..(6): D form
  MISC_FEATURE (11)..(11): Xaa = Pro reduced from amino acid to amino alcohol
YRCEHFRWCKX

SEQ ID NO: 123 - protein, 11 aa, Artificial
  Synthetic construct
  MOD_RES (1)..(1): ACETYLATION
  DISULFID (3)..(9)
  MOD_RES (6)..(6): D form
  MOD_RES (11)..(11): AMIDATION
YRCEHFRWCRF

SEQ ID NO: 124 - protein, 9 aa, Artificial
  Synthetic construct
  MOD_RES (1)..(1): ACETYLATION
  MISC_FEATURE (2)..(2): Xaa = citrulline
  DISULFID (3)..(9)
  MOD_RES (6)..(6): D form
  MOD_RES (9)..(9): AMIDATION
YXCEHFRWC

SEQ ID NO: 125 - protein, 9 aa, Artificial
  Synthetic construct
  MOD_RES (1)..(1): ACETYLATION
  MISC_FEATURE (2)..(2): Xaa = citrulline
  DISULFID (3)..(9)
  MOD_RES (5)..(5): 1-methyl substituted
  MOD_RES (6)..(6): D form
  MOD_RES (9)..(9): AMIDATION
YXCEHFRWC

SEQ ID NO: 126 - protein, 9 aa, Artificial
  Synthetic construct
  MOD_RES (1)..(1): ACETYLATION
  MISC_FEATURE (2)..(2): Xaa = homoarginine
  DISULFID (3)..(9)
  MOD_RES (6)..(6): D form
  MOD_RES (9)..(9): AMIDATION
YXCEHFRWC

SEQ ID NO: 127 - protein, 9 aa, Artificial
  Synthetic construct
  MOD_RES (1)..(1): ACETYLATION
  MISC_FEATURE (2)..(2): Xaa = 1-beta-homoarginine
  DISULFID (3)..(9)
  MOD_RES (6)..(6): D form
  MOD_RES (9)..(9): AMIDATION
YXCEHFRWC

SEQ ID NO: 128 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YKCEHFRWC

SEQ ID NO: 129 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YSCEHFRWC

SEQ ID NO: 130 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YVCEHFRWC

SEQ ID NO: 131 - protein, 9 aa, Artificial
  Synthetic construct
  MOD_RES (1)..(1): N-succinyl substituted
  DISULFID (3)..(9)
  MOD_RES (6)..(6): D form
YRCEHFRWC

SEQ ID NO: 132 - protein, 6 aa, Artificial
  Synthetic construct
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 133 - protein, 6 aa, Artificial
  Synthetic construct
  DISULFID (1)..(6)
  MISC_FEATURE (1)..(1): Xaa = homocysteine
  MOD_RES (3)..(3): D form
XHFRWC

SEQ ID NO: 134 - protein, 6 aa, Artificial
  Synthetic construct
  DISULFID (1)..(6)
  MISC_FEATURE (1)..(1): Xaa = homocysteine
  MOD_RES (3)..(3): 4-fluoro substituted, D form
  MOD_RES (6)..(6): AMIDATION
XHFRWC

SEQ ID NO: 135 - protein, 6 aa, Artificial
  Synthetic construct
  DISULFID (1)..(6)
  MISC_FEATURE (1)..(1): Xaa = homocysteine
  MOD_RES (3)..(3): 4-chloro substituted, D form
  MOD_RES (6)..(6): AMIDATION
XHFRWC

SEQ ID NO: 136 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (6)..(6): AMIDATION
XHFRWC

SEQ ID NO: 137 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 138 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): D form
XHFRWC

SEQ ID NO: 139 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): 4-fluoro substituted, D form
  MOD_RES (6)..(6): AMIDATION
XHFRWC

SEQ ID NO: 140 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): 4-chloro substituted, D form
  MOD_RES (6)..(6): AMIDATION
XHFRWC

SEQ ID NO: 141 - protein, 6 aa, Artificial
  Synthetic construct
  MOD_RES (1)..(1): N-cyclopropanecarbonyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 142 - protein, 6 aa, Artificial
  Synthetic construct
  MOD_RES (1)..(1): N-cyclobutanecarbonyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 143 - protein, 6 aa, Artificial
  Synthetic construct
  MOD_RES (1)..(1): N-cyclopentanecarbonyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 144 - protein, 6 aa, Artificial
  Synthetic construct
  MOD_RES (1)..(1): N-cyclohexanecarbonyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 145 - protein, 6 aa, Artificial
  Synthetic construct
  MOD_RES (1)..(1): N-hexanoyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 146 - protein, 6 aa, Artificial
  Synthetic construct
  MOD_RES (1)..(1): N-benzoyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 147 - protein, 6 aa, Artificial
  Synthetic construct
  MOD_RES (1)..(1): 4-phenylbutyrylsubstituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 148 - protein, 6 aa, Artificial
  Synthetic construct
  MOD_RES (1)..(1): 3-guanidinopropionyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 149 - protein, 6 aa, Artificial
  Synthetic construct
  MOD_RES (1)..(1): 5-guanidinovaleryl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 150 - protein, 6 aa, Artificial
  Synthetic construct
  MOD_RES (1)..(1): N-phenylsulfonyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 151 - protein, 6 aa, Artificial
  Synthetic construct
  MOD_RES (1)..(1): N-(2-naphthalenesulfonyl) substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 152 - protein, 6 aa, Artificial
  Synthetic construct
  MOD_RES (1)..(1): N-(4-phenylsulfonamido-4-oxo-butyryl) substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 153 - protein, 7 aa, Artificial
  Synthetic construct
  DISULFID (2)..(7)
  MISC_FEATURE (2)..(2): Xaa = homocysteine
  MOD_RES (4)..(4): D form
  MOD_RES (7)..(7): AMIDATION
RXHFRWC

SEQ ID NO: 154 - protein, 7 aa, Artificial
  Synthetic construct
  MOD_RES (1)..(1): D form
  DISULFID (2)..(7)
  MISC_FEATURE (2)..(2): Xaa = homocysteine
  MOD_RES (4)..(4): D form
  MOD_RES (7)..(7): AMIDATION
RXHFRWC

SEQ ID NO: 155 - protein, 7 aa, Artificial
  Synthetic construct
  DISULFID (2)..(7)
  MISC_FEATURE (2)..(2): Xaa = homocysteine
  MOD_RES (4)..(4): D form
RXHFRWC

SEQ ID NO: 156 - protein, 7 aa, Artificial
  Synthetic construct
  DISULFID (2)..(7)
  MISC_FEATURE (2)..(2): Xaa = homocysteine
  MISC_FEATURE (3)..(3): 1-methyl substituted
  MOD_RES (4)..(4): D form
  MOD_RES (7)..(7): AMIDATION
RXHFRWC

SEQ ID NO: 157 - protein, 7 aa, Artificial
  Synthetic construct
  DISULFID (2)..(7)
  MISC_FEATURE (2)..(2): Xaa = homocysteine
  MOD_RES (3)..(3): 1-methyl substituted, D form
  MOD_RES (4)..(4): D form
  MOD_RES (7)..(7): AMIDATION
RXHFRWC

SEQ ID NO: 158 - protein, 7 aa, Artificial
  Synthetic construct
  MOD_RES (1)..(1): ACETYLATION
  DISULFID (2)..(7)
  MISC_FEATURE (2)..(2): Xaa = homocysteine
  MOD_RES (4)..(4): D form
  MOD_RES (7)..(7): AMIDATION
RXHFRWC

SEQ ID NO: 159 - protein, 7 aa, Artificial
  Synthetic construct
  MOD_RES (1)..(1): ACETYLATION
  DISULFID (2)..(7)
  MISC_FEATURE (2)..(2): Xaa = homocysteine
  MOD_RES (4)..(4): D form
RXHFRWC

SEQ ID NO: 160 - protein, 7 aa, Artificial
  Synthetic construct
  MOD_RES (1)..(1): ACETYLATION
  MISC_FEATURE (1)..(1): Xaa = norleucine
  DISULFID (2)..(7)
  MISC_FEATURE (2)..(2): Xaa = homocysteine
  MOD_RES (4)..(4): D form
  MOD_RES (7)..(7): AMIDATION
XXHFRWC

SEQ ID NO: 161 - protein, 7 aa, Artificial
  Synthetic construct
  MOD_RES (1)..(1): phenylsulfonyl substituted
  DISULFID (2)..(7)
  MISC_FEATURE (2)..(2): Xaa = homocysteine
  MOD_RES (4)..(4): D form
  MOD_RES (7)..(7): AMIDATION
GXHFRWC

SEQ ID NO: 162 - protein, 8 aa, Artificial
  Synthetic construct
  DISULFID (3)..(8)
  MISC_FEATURE (3)..(3): Xaa = homocysteine
  MOD_RES (5)..(5): D form
  MOD_RES (8)..(8): AMIDATION
YRXHFRWC

SEQ ID NO: 163 - protein, 8 aa, Artificial
  Synthetic construct
  DISULFID (3)..(8)
  MISC_FEATURE (3)..(3): Xaa = homocysteine
  MOD_RES (5)..(5): D form
YRXHFRWC

SEQ ID NO: 164 - protein, 8 aa, Artificial
  Synthetic construct
  MOD_RES (1)..(1): ACETYLATION
  DISULFID (3)..(8)
  MISC_FEATURE (3)..(3): Xaa = homocysteine
  MOD_RES (5)..(5): D form
  MOD_RES (8)..(8): AMIDATION
YRXHFRWC

SEQ ID NO: 165 - protein, 8 aa, Artificial
  Synthetic construct
  MOD_RES (1)..(1): ACETYLATION
  DISULFID (3)..(8)
  MISC_FEATURE (3)..(3): Xaa = homocysteine
  MOD_RES (5)..(5): D form
YRXHFRWC

SEQ ID NO: 166 - protein, 9 aa, Artificial
  Synthetic construct
  MOD_RES (1)..(1): ACETYLATION
  DISULFID (3)..(9)
  MISC_FEATURE (3)..(3): Xaa = homocysteine
  MOD_RES (6)..(6): D form
  MOD_RES (9)..(9): AMIDATION
YRXEHFRWC

SEQ ID NO: 167 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): beta-cyclohexyl substituted, D form
  MOD_RES (6)..(6): AMIDATION
XHARWC

SEQ ID NO: 168 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 169 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): 4-chloro substituted, D form
  MOD_RES (6)..(6): AMIDATION
  MISC_FEATURE (6)..(6): Xaa = penicillamine
XHFRWX

SEQ ID NO: 170 - protein, 6 aa, Artificial
  Synthetic construct
  MOD_RES (1)..(1): N-hexanoyl substituted
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 171 - protein, 6 aa, Artificial
  Synthetic construct
  MOD_RES (1)..(1): N-cyclopentanecarbonyl substituted
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 172 - protein, 6 aa, Artificial
  Synthetic construct
  MOD_RES (1)..(1): N-cyclohexanecarbonyl substituted
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 173 - protein, 6 aa, Artificial
  Synthetic construct
  MOD_RES (1)..(1): N-benzoyl substituted
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 174 - protein, 6 aa, Artificial
  Synthetic construct
  MOD_RES (1)..(1): 4-phenylbutyryl substituted
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 175 - protein, 6 aa, Artificial
  Synthetic construct
  MOD_RES (1)..(1): N-phenylsulfonyl substituted
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 176 - protein, 6 aa, Artificial
  Synthetic construct
  MOD_RES (1)..(1): (4-benzenesulfonamide)butyryl substituted
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 177 - protein, 7 aa, Artificial
  Synthetic construct
  MOD_RES (1)..(1): ACETYLATION
  MISC_FEATURE (1)..(1): Xaa = norleucine
  DISULFID (2)..(7)
  MISC_FEATURE (2)..(2): Xaa = homocysteine
  MOD_RES (4)..(4): D form
  MOD_RES (7)..(7): AMIDATION
  MISC_FEATURE (7)..(7): Xaa = penicillamine
XXHFRWX

SEQ ID NO: 178 - protein, 7 aa, Artificial
  Synthetic construct
  MOD_RES (1)..(1): N-phenylsulfonyl substituted
  DISULFID (2)..(7)
  MISC_FEATURE (2)..(2): Xaa = homocysteine
  MOD_RES (4)..(4): D form
  MOD_RES (7)..(7): AMIDATION
  MISC_FEATURE (7)..(7): Xaa = penicillamine
GXHFRWX

SEQ ID NO: 179 - protein, 6 aa, Artificial
  Synthetic construct
  MISC_FEATURE (1)..(1): Xaa = desamino Cys
  DISULFID (1)..(6)
  MOD_RES (3)..(3): D form
  MOD_RES (6)..(6): AMIDATION
  MISC_FEATURE (6)..(6): Xaa = homocysteine
XHFRWX

SEQ ID NO: 180 - protein, 6 aa, Artificial
  Synthetic construct
  DISULFID (1)..(6)
  MOD_RES (3)..(3): D form
  MOD_RES (6)..(6): AMIDATION
  MISC_FEATURE (6)..(6): Xaa = homocysteine
CHFRWX

SEQ ID NO: 181 - protein, 6 aa, Artificial
  Synthetic construct
  DISULFID (1)..(6)
  MOD_RES (3)..(3): 4-fluoro substituted, D form
  MOD_RES (6)..(6): AMIDATION
  MISC_FEATURE (6)..(6): Xaa = homocysteine
CHFRWX

SEQ ID NO: 182 - protein, 6 aa, Artificial
  Synthetic construct
  DISULFID (1)..(6)
  MOD_RES (3)..(3): 4-chloro substituted, D form
  MOD_RES (6)..(6): AMIDATION
  MISC_FEATURE (6)..(6): Xaa = homocysteine
CHFRWX

SEQ ID NO: 183 - protein, 6 aa, Artificial
  Synthetic construct
  MOD_RES (1)..(1): ACETYLATION
  DISULFID (1)..(6)
  MOD_RES (3)..(3): D form
  MOD_RES (6)..(6): AMIDATION
  MISC_FEATURE (6)..(6): Xaa = homocysteine
CHFRWX

SEQ ID NO: 184 - protein, 6 aa, Artificial
  Synthetic construct
  MOD_RES (1)..(1): ACETYLATION
  DISULFID (1)..(6)
  MOD_RES (3)..(3): 4-fluoro substituted, D form
  MOD_RES (6)..(6): AMIDATION
  MISC_FEATURE (6)..(6): Xaa = homocysteine
CHFRWX

SEQ ID NO: 185 - protein, 6 aa, Artificial
  Synthetic construct
  MOD_RES (1)..(1): ACETYLATION
  DISULFID (1)..(6)
  MOD_RES (3)..(3): 4-chloro substituted, D form
  MOD_RES (6)..(6): AMIDATION
  MISC_FEATURE (6)..(6): Xaa = homocysteine
CHFRWX

SEQ ID NO: 186 - protein, 7 aa, Artificial
  Synthetic construct
  DISULFID (2)..(7)
  MOD_RES (4)..(4): D form
  MOD_RES (7)..(7): AMIDATION
  MISC_FEATURE (7)..(7): Xaa = homocysteine
RCHFRWX

SEQ ID NO: 187 - protein, 7 aa, Artificial
  Synthetic construct
  DISULFID (2)..(7)
  MOD_RES (4)..(4): 4-fluoro substituted, D form
  MOD_RES (7)..(7): AMIDATION
  MISC_FEATURE (7)..(7): Xaa = homocysteine
RCHFRWX

SEQ ID NO: 188 - protein, 7 aa, Artificial
  Synthetic construct
  DISULFID (2)..(7)
  MOD_RES (4)..(4): 4-chloro substituted, D form
  MOD_RES (7)..(7): AMIDATION
  MISC_FEATURE (7)..(7): Xaa = homocysteine
RCHFRWX

SEQ ID NO: 189 - protein, 7 aa, Artificial
  Synthetic construct
  MOD_RES (1)..(1): ACETYLATION
  DISULFID (2)..(7)
  MOD_RES (4)..(4): D form
  MOD_RES (7)..(7): AMIDATION
  MISC_FEATURE (7)..(7): Xaa = homocysteine
RCHFRWX

SEQ ID NO: 190 - protein, 7 aa, Artificial
  Synthetic construct
  MOD_RES (1)..(1): ACETYLATION
  DISULFID (2)..(7)
  MOD_RES (4)..(4): 4-fluoro substituted, D form
  MOD_RES (7)..(7): AMIDATION
  MISC_FEATURE (7)..(7): Xaa = homocysteine
RCHFRWX

SEQ ID NO: 191 - protein, 7 aa, Artificial
  Synthetic construct
  MOD_RES (1)..(1): ACETYLATION
  DISULFID (2)..(7)
  MOD_RES (4)..(4): 4-chloro substituted, D form
  MOD_RES (7)..(7): AMIDATION
  MISC_FEATURE (7)..(7): Xaa = homocysteine
RCHFRWX

SEQ ID NO: 192 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
  MISC_FEATURE (9)..(9): Xaa = homocysteine
YRCEHFRWX

SEQ ID NO: 193 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = homocysteine
XHFRWX

SEQ ID NO: 194 - protein, 7 aa, Artificial
  Synthetic construct
  DISULFID (2)..(7)
  MISC_FEATURE (2)..(2): Xaa = homocysteine
  MOD_RES (4)..(4): D form
  MOD_RES (7)..(7): AMIDATION
  MISC_FEATURE (7)..(7): Xaa = homocysteine
RXHFRWX

SEQ ID NO: 195 - protein, 7 aa, Artificial
  Synthetic construct
  MOD_RES (1)..(1): ACETYLATION
  DISULFID (2)..(7)
  MISC_FEATURE (2)..(2): Xaa = homocysteine
  MOD_RES (4)..(4): D form
  MOD_RES (7)..(7): AMIDATION
  MISC_FEATURE (7)..(7): Xaa = homocysteine
RXHFRWX

SEQ ID NO: 196 - protein, 8 aa, Artificial
  Synthetic construct
  MOD_RES (1)..(1): ACETYLATION
  DISULFID (3)..(8)
  MISC_FEATURE (3)..(3): Xaa = homocysteine
  MOD_RES (5)..(5): D form
  MOD_RES (8)..(8): AMIDATION
  MISC_FEATURE (8)..(8): Xaa = homocysteine
YRXHFRWX

SEQ ID NO: 197 - protein, 9 aa, Artificial
  Synthetic construct
  MOD_RES (1)..(1): ACETYLATION
  DISULFID (3)..(9)
  MISC_FEATURE (3)..(3): Xaa = homocysteine
  MOD_RES (6)..(6): D form
  MOD_RES (9)..(9): AMIDATION
  MISC_FEATURE (9)..(9): Xaa = homocysteine
YRXEHFRWX

SEQ ID NO: 198 - protein, 6 aa, Artificial
  Synthetic construct
  MOD_RES (1)..(1): ACETYLATION
  DISULFID (1)..(6): S-CH2-S linkage
  MOD_RES (3)..(3): D form
  MOD_RES (6)..(6): AMIDATION
CHFRWC

SEQ ID NO: 199 - protein, 9 aa, Artificial
  Synthetic construct
  MISC_FEATURE (1)..(1): Xaa = Arg, Tyr-Arg, Tyr-beta-Arg, or is absent
  MISC_FEATURE (1)..(1): Xaa = a modified amino acid including Arg, citrulline, homoarginine, Leu, Lys, N-isopropyl-Lys, norleucine, ornithine, or Val
  MISC_FEATURE (1)..(1): Xaa = a modified group including Tyr-Arg, Tyr-citrulline, Cya-Arg, Tyr-homoarginine, Tyr-1-beta- homoarginine, Tyr-Lys, Tyr-Ser, or Tyr-Val
  DISULFID (2)..(8): S-S or S-CH2-S disulfide bridge
  MISC_FEATURE (2)..(2): Xaa = Cys, homocysteine, or desamino-cysteine; may be D or L form
  MISC_FEATURE (3)..(3): Xaa = Glu, Gln, Asp, Asn, Ala, Gly, Thr, Ser, Pro, Met, Ile, Val, Arg, His, Tyr, Trp, Phe, Lys, Leu, cysteic acid, or is absent
  MISC_FEATURE (4)..(4): Xaa = His, modified His, or modified Ala; D or L form
  MISC_FEATURE (5)..(5): Xaa = Phe, modified Phe, or modified Ala; D or L form
  MISC_FEATURE (6)..(6): Xaa = Arg or modified Arg; D or L form
  MISC_FEATURE (8)..(8): Xaa = Cys, homocysteine, or modified cysteine or homocysteine (such as amide, alcohol, or penicillamine)
  MISC_FEATURE (9)..(9): Xaa = Ser-Pro-NH2, Lys-Pro-NH2, Ser-OH, Ser-Pro-OH, Lys-OH, Ser alcohol, Ser-Pro alcohol, Arg-Phe-NH2, Glu-NH2, or is absent
XXXXXXWXX

SEQ ID NO: 200 - protein, 8 aa, Artificial
  Synthetic construct
  MISC_FEATURE (1)..(1): Xaa = Arg, Tyr-Arg, Tyr-beta-Arg, or is absent
  MISC_FEATURE (1)..(1): Xaa = a modified amino acid including Arg, citrulline, homoarginine, Leu, Lys, N-isopropyl-Lys, norleucine, ornithine, or Val
  MISC_FEATURE (1)..(1): Xaa = a modified group including Tyr-Arg, Tyr-citrulline, Cya-Arg, Tyr-homoarginine, Tyr-1-beta- homoarginine, Tyr-Lys, Tyr-Ser, or Tyr-Val
  DISULFID (2)..(8)
  MISC_FEATURE (2)..(2): Xaa = Cys or homocysteine
  MISC_FEATURE (3)..(3): Xaa = Glu, Gln, Asp, Asn, Ala, Gly, Thr, Ser, Pro, Met, Ile, Val, Arg, His, Tyr, Trp, Phe, Lys, Leu, cysteic acid, or is absent
  MOD_RES (4)..(4): His may be optionally substituted, D or L form
  MOD_RES (5)..(5): Phe may be optionally substituted, D or L form
  MISC_FEATURE (8)..(8): Xaa = Cys, homocysteine, or modified cysteine or homocysteine such as amide
  MISC_FEATURE (9)..(9): Xaa = Ser-Pro-NH2, Lys-Pro-NH2, Ser-OH, Ser-Pro-OH, Lys-OH, Ser alcohol, Ser-Pro alcohol, Arg-Phe-NH2, Glu-NH2, or is absent
XXXHFRXX

SEQ ID NO: 201 - protein, 9 aa, Artificial
  Synthetic construct
  MISC_FEATURE (1)..(1): Xaa = Arg, Tyr-Arg, Tyr-beta-Arg, or is absent
  MISC_FEATURE (1)..(1): Xaa = a modified amino acid including Arg, citrulline, homoarginine, Leu, Lys, N-isopropyl-Lys, norleucine, ornithine, or Val
  MISC_FEATURE (1)..(1): Xaa = a modified group including Tyr-Arg, Tyr-citrulline, Tyr-homoarginine, Tyr-1-beta-homoarginine, Tyr-Lys, Tyr-Ser, or Tyr-Val
  DISULFID (2)..(8)
  MISC_FEATURE (2)..(2): Xaa = Cys or homocysteine
  MISC_FEATURE (3)..(3): Xaa = Glu, Gln, Asp, Asn, Ala, Gly, Thr, Ser, Pro, Met, Ile, Val, Arg, His, Tyr, Trp, Phe, or is absent
  MOD_RES (4)..(4): His may be optionally substituted, D or L form
  MOD_RES (5)..(5): Phe may be optionally substituted, D or L form
  MISC_FEATURE (8)..(8): Xaa = Cys, homocysteine, or modified cysteine or homocysteine such as amide
  MISC_FEATURE (9)..(9): Xaa = Ser-Pro-NH2, Lys-Pro-NH2, Ser-OH, Ser-Pro-OH, Lys-Pro-OH, Arg-Phe-NH2, Glu-NH2, or is absent
XXXHFRWXX

(19) United States
(12) Patent Application Publication
(10) Pub. No.: US 2007/0105759 A1
(43) Pub. Date: May 10, 2007

(54) Melanocortin receptor 4 (mc4) agonists and their uses

(75) Inventors: David Benjamin Flora, Greenfield, IN (US); Mark Louis Heiman, Indianapolis, IN (US); JeAnne L. Hertel, Indianapolis, IN (US); Hansen M. Hsiung, Indianapolis, IN (US); John P. Mayer, Indianapolis, IN (US); David L. Smiley, Greenfield, IN (US); Liang Zeng Yan, Carmel, IN (US); Lianshan Zhang, Carmel, IN (US)

Correspondence Address: ELI LILLY & COMPANY, PATENT DIVISION, P.O. BOX 6288, INDIANAPOLIS, IN, 46206-6288, US

(73) Assignee: ELI LILLY AND COMPANY, INDIANAPOLIS, IN (US)

(21) Appl. No.: 10/556,689

(22) PCT Filed: Jun. 17, 2004
(86) PCT No.: PCT/US04/16625

§ 371 Date: Nov. 15, 2005

Related U.S. Application Data
(60) Provisional application No. 60/479,740, filed on Jun. 19, 2003.
(60) Provisional application No. 60/557,347, filed on Mar. 29, 2004.
(60) Provisional application No. 60/570,676, filed on May 13, 2004.
(60) Provisional application No. 60/570,737, filed on May 13, 2004.

(51) Int. Cl. A61K 38/12; C07K 7/64

(52) U.S. Cl. 514/9; 530/317

(57) ABSTRACT

The present invention relates to peptide agonists of the MC4 receptor, and as such are useful in the treatment of disorders responsive to the activation of this receptor, such as obesity, diabetes mellitus and male and/or female sexual dysfunction.

The present invention relates to peptide agonists of the MC4 receptor and as such are useful in the treatment of disorders responsive to the activation of this receptor, such as obesity, diabetes mellitus, and male and/or female sexual dysfunction.

The proopiomelanocortin (POMC) gene encodes a 31-36 kDa pre-prohormone, from which seven mature peptide hormones are derived. POMC processing occurs in a tissue specific manner yielding four distinct melanocortin peptides: adrenocorticotropic hormone (ACTH), α-melanocyte stimulating hormone (α-MSH), β-MSH, and γ-MSH.

Five melanocortin receptors have thus far been identified and are referred to herein as MC1, MC2, MC3, MC4, and MC5. MC1, whose primary endogenous ligand is α-MSH, is associated with pigmentation. MC2, whose primary endogenous ligand is ACTH, is associated with steroidogenesis. MC2 is distinctly different from the other melanocortin receptors and is not expected to interact with endogenous or synthetic MSHs other than ACTH or analogues thereof (Schiöth et al., Life Sciences 59(10):797-801, 1996). MC5 is believed to have two primary ligands, α-MSH and ACTH, and is associated with exocrine Amenand sebaceous gland lipid secretion.

Diverse lines of evidence, including genetic and pharmacological data obtained in rodents and humans, support a role for the MC4 receptor in the regulation of energy homeostasis, specifically regulating food intake and metabolism. The distribution of MC4 receptors in the brain correlates well with the areas in the brain which show high sensitivity to melanocortin-mediated feeding behavior (MacNeil et al., Eur. J. Pharm. 440(2-3):141-57, 2002). In addition, the MC4 receptor is believed to be significantly involved in regulating body weight as evidenced by the fact that Mc4r−/−mice are obese, and humans with mutations in the melanocortin MC4 receptor gene are obese. Thus, MC4 receptor agonists may be beneficial for the treatment of obesity.

The development of selective peptide agonists for melanocortin receptors has closely followed the identification of the various melanocortin receptor subtypes and their perceived primary ligands. Id. α-MSH, a 13-amino acid peptide, is a non-selective agonist at four melanocortin receptors, MC1 and MC3-MC5. NDP-αMSH is a more potent, protease resistant, but still non-selective analogue of α-MSH.

The lactam derived from the 4-10 fragment of NDP-αMSH, known as MTII, is even more potent in vivo than NDP-α-MSH but is non-selective. Replacement of the D-Phe with D-(2′)Na1 in MTII, yielded a high affinity antagonist for MC3 and MC4 that is an agonist for the MC1 and MC5 receptors. This peptide is known as SHU9119.

Although many peptides cyclized via disulfide bridges are MC4 receptor agonists, several are MC4 receptor antagonists with moderate selectivity over the MC3 receptor. The peptide HS014 is a partial agonist at the MC1 and MC5 receptors, while the peptide HS024 does not display agonist activity at the MC1 and MC3 receptors. In addition, PCT Publication No. WO 00/35952 discloses certain peptides cyclized via disulfide bridges having utility as MC4 agonists.

Despite the progress discussed above and elsewhere, there continues to be a need for MC4 agonists with pharmaceutically desirable selectivity, potency and efficacy, for use as a pharmaceutical, in particular, for the treatment of obesity. Especially desired are MC4 agonists with a clinically desirable pharmacology and safety profile.

Obesity

Obesity, and especially upper body obesity, is a common and very serious public health problem in the United States and throughout the world. According to recent statistics, more than 25% of the United States population and 27% of the Canadian population are overweight. Kuczmarski, Amer. J. of Clin. Nutr. 55:495S-502S, 1992; Reeder et al., Can. Med. Assn. J., 23:226-33, 1992. Upper body obesity is the strongest risk factor known for type II diabetes mellitus, and is a strong risk factor for cardiovascular disease and cancer as well. Recent estimates for the medical cost of obesity are $150,000,000,000 worldwide. The problem has become serious enough that the surgeon general has begun an initiative to combat the ever-increasing adiposity rampant in American society.

Male and/or Female Sexual Dysfunction

The MC4 receptor appears to play role in other physiological functions as well, namely controlling grooming behavior, erection, and blood pressure. “Female sexual dysfunction” encompasses, without limitation, conditions such as a lack of sexual desire and related arousal disorders, inhibited orgasm, lubrication difficulties, and vaginismus.

“Erectile dysfunction” is a disorder involving the failure of a male mammal to achieve erection, ejaculation, or both. Symptoms of erectile dysfunction include an inability to achieve or maintain an erection, ejaculatory failure, premature ejaculation, and inability to achieve an orgasm. An increase in erectile dysfunction is often associated with age and is generally caused by a physical disease or as a side effect of drug treatment. The term “impotence” is often times employed to describe this prevalent condition. Synthetic melanocortin receptor agonists have been found to initiate erections in men with psychogenic erectile dysfunction (Wessells et al., “Synthetic Melanotropic Peptide Initiates Erections in Men With Psychogenic Erectile Dysfunction: Double-Blind, Placebo Controlled Crossover Study,” J. Urol., 160:389-93, 1998). Activation of melanocortin receptors of the brain appears to cause normal stimulation of sexual arousal. Evidence for the involvement of the MC4 receptor in male and/or female sexual dysfunction is detailed in WO 00/74670.

Diabetes

Diabetes is a disease in which a mammal's ability to regulate glucose levels in the blood is impaired because the mammal has a reduced ability to convert glucose to glycogen for storage in muscle and liver cells. In Type I diabetes, this reduced ability to store glucose is caused by reduced insulin production. “Type II Diabetes” or “non-insulin dependent diabetes mellitus” (NIDDM) is a form of diabetes which is due to a profound resistance to insulin stimulating or regulatory effect on glucose and lipid metabolism in the main insulin-sensitive tissues: muscle, liver, and adipose tissue. This resistance to insulin responsiveness results in insufficient insulin activation of glucose uptake, oxidation, and storage in muscle and inadequate insulin repression of lipolysis in adipose tissue and of glucose production and secretion in liver. When these cells become desensitized to insulin, the body tries to compensate by producing abnormally high levels of insulin, and hyperinsulemia results. Hyperinsulemia is associated with hypertension and elevated body weight. Since insulin is involved in promoting the cellular uptake of glucose, amino acids, and triglycerides from the blood by insulin sensitive cells, insulin insensitivity can result in elevated levels of triglycerides and LDL which are risk factors in cardiovascular diseases. The constellation of symptoms, which includes hyperinsulemia, combined with hypertension, elevated body weight, elevated triglycerides and elevated LDL, is known as Syndrome X.

Applicants have discovered compounds that have an unexpectedly high affinity for the MC4 receptor and are selective for the MC4 receptor over other melanocortin receptor subtypes.

The present invention is directed to compounds represented by the following Structural Formula I (SEQ ID NO:199): 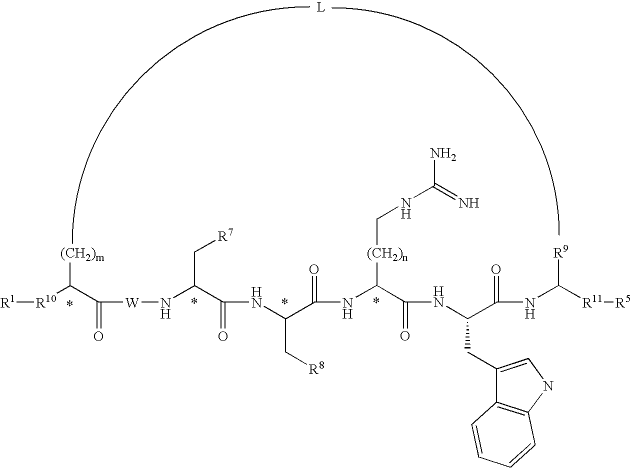
and pharmaceutically acceptable salts thereof, wherein
W is Glu, Gln, Asp, Asn, Ala, Gly, Thr, Ser, Pro, Met, Ile, Val, Arg, His, Tyr, Trp, Phe, Lys, Leu, Cya, or is absent;
R¹ is —H, —C(O)CH₃, —C(O)(CH₂)₁₋₄CH₃, —C(O)(CH₂)₁₋₄NHC(NH)NH₂, Tyr-βArg-, Ac-Tyr-β-hArg-, gluconoyl-Tyr-Arg-, Ac-diaminobutyryl-, Ac-diaminopropionyl-, N-propionyl-, N-butyryl-, N-valeryl-, N-methyl-Tyr-Arg-, N-glutaryl-Tyr-Arg-, N-succinyl-Tyr-Arg-, R⁶—SO₂NHC(O)CH₂CH₂C(O)—, R⁶—SO₂NHC(O)CH₂CH₂C(O)Arg-, R⁶—SO₂NHCH₂CH₂CH₂C(O)—, C₃-C₇ cycloalkylcarbonyl, phenylsulfonyl, C₈-C₁₄ bicyclic arylsulfonyl, phenyl-(CH₂)_{q}C(O)—, C₈-C₁₄ bicyclic aryl-(CH₂)_{q}C(O)—, 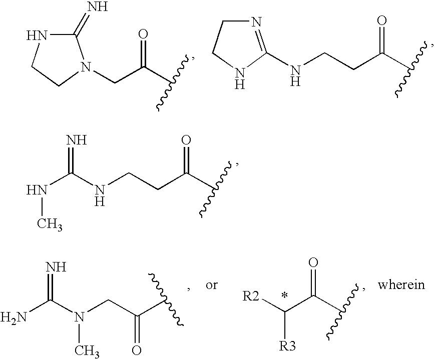
R² is —H, —NH₂, —NHC(O)CH₃, —NHC(O)(CH₂)₁₋₄CH₃, —NH-TyrC(O)CH₃, R⁶SO₂NH—, Ac-Cya-NH—, Tyr-NH—, HO—(C₆H₅)—CH₂CH₂C(O)NH—, or CH₃—(C₆H₅)—C(O)CH₂CH₂C(O)NH—;
R³ is C₁-C₄ straight or branched alkyl, NH₂—CH₂—(CH₂)_{q}—, HO—CH₂—, (CH₃)₂CHNH(CH₂)₄—, R⁶(CH₂)_{q}—, R⁶SO₂NH—, Ser, Ile, 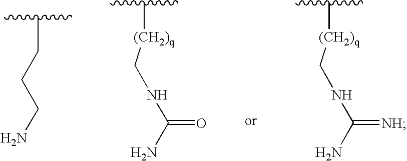
q is 0, 1, 2, or 3;
R⁶ is a phenyl or C₈-C₁₄ bicyclic aryl;
m is 1 or 2;
n is 1, 2, 3, or 4;
R⁹ is (CH₂)ₚ or (CH₃)₂C—;
p is 1 or 2;
R¹⁰ is NH— or is absent;
R⁷ is a 5- or 6-membered heteroaryl or a 5- or 6-membered heteroaryl ring optionally substituted with R⁴;
R⁴ is H, C₁-C₄ straight or branched alkyl, phenyl, benzyl, or (C₆H₅)—CH₂—O—CH₂—;
R⁸ is phenyl, a phenyl ring optionally substituted with X, or cyclohexyl;
X is H, Cl, F, Br, methyl, or methoxy;
R¹¹ is —C(O) or —CH₂;
R⁵ is —NH₂, —OH, glycinol, NH₂-Pro-Ser-, NH₂-Pro-Lys-, HO-Ser-, HO-Pro-Ser-, HO-Lys-, -Ser alcohol, -Ser-Pro alcohol, -Lys-Pro alcohol, HOCH₂CH₂—O—CH₂CH₂NH—, NH₂-Phe-Arg-, NH₂-Glu-, NH₂CH₂RCH₂NH—, RHN—, or RO— where R is a C₁-C₄ straight or branched alkyl; and
L is —S—S— or —S—CH₂—S—.

In a preferred embodiment, the invention is directed to compounds represented by the following Structural Formula II (SEQ ID NO:200): 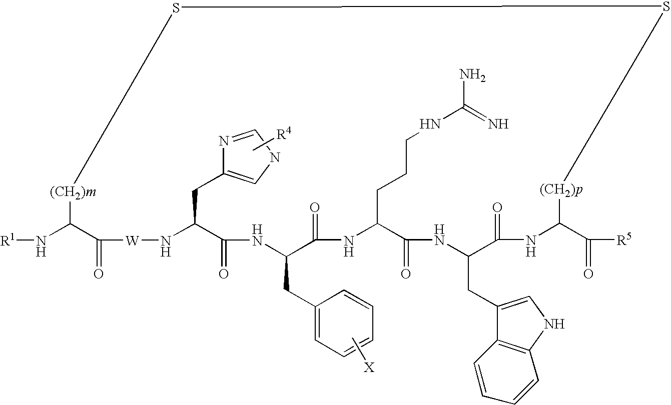
and pharmaceutically acceptable salts thereof, wherein
W is a single bond, Glu, Gln, Asp, Asn, Ala, Gly, Thr, Ser, Pro, Met, Ile, Val, Arg, His, Tyr, Trp, or Phe;
R¹ is —H, —C(O)CH₃, —C(O)(CH₂)₁₋₄CH₃, —C(O)(CH₂)₁₋₄—NHC(NH)NH₂, Tyr-βArg, gluconoyl-Tyr-Arg, Ac-Dab, Ac-Dap, N-succinyl-Tyr-Arg, N-propionyl, N-valeryl, N-glutaryl-Tyr-Arg, N-butyryl, 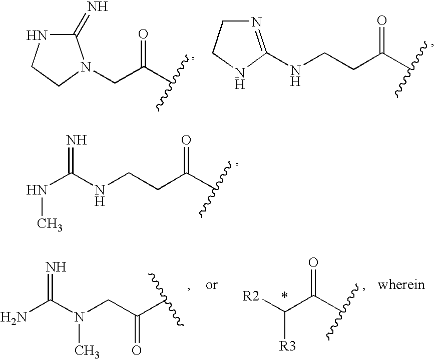
R² is —H, —NH₂, —NHC(O)CH₃, —NHC(O)(CH₂)₁₋₄CH₃, or —NH-TyrC(O)CH₃;
R³ is C₁-C₄ straight or branched alkyl, Ser, Ile, 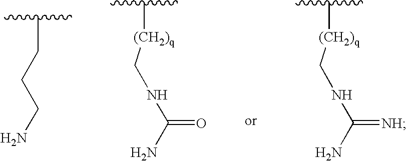
q is 0, 1, 2, or 3;
m is 1 or 2;
p is 1 or 2;
R⁴ is H or C₁-C₄ straight or branched alkyl;
X is H, Cl, F, Br, methyl, or methoxy; and
R⁵ is —NH₂, —OH, glycinol, -Ser-Pro-NH₂, -Lys-Pro-NH₂, -Ser-OH, -Ser-Pro-OH, -Lys-Pro-OH-Arg-Phe-NH₂, -Glu-NH₂, —NHR, or —OR, where R is a C₁-C₄ straight or branched alkyl.

In another embodiment, the present invention is directed to compounds represented by Structural Formula II with the proviso that the combination of R₂=Tyr, R₃=Arg, W=Glu, R₄=H, X=H, m=1, p=1, and R₅=NH₂ is specifically excluded.

Another preferred embodiment of the present invention includes compounds of Structural Formula III (SEQ ID NO:201):

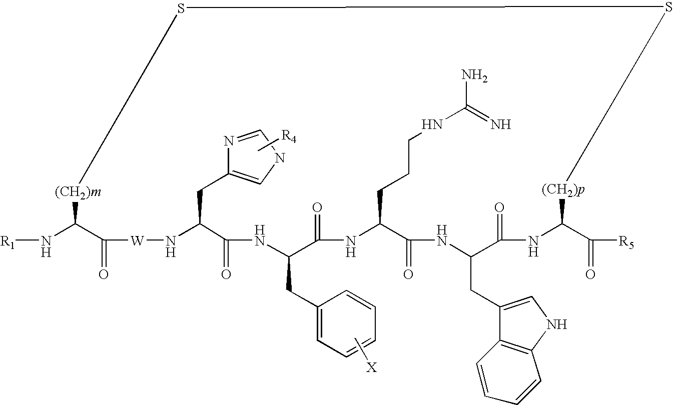

and pharmaceutically acceptable salts thereof, wherein
W is Glu, Gln, Asp, Asn, Ala, Gly, Thr, Ser, Pro, Met, Ile, Val, Arg, His, Tyr, Trp, Phe, Lys, Leu, Cya, or is absent;
R¹ is —H, —C(O)CH₃, —C(O)(CH₂)₁₋₄CH₃, —C(O)(CH₂)₁₋₄NHC(NH)NH₂, Tyr-βArg-, Ac-Tyr-β-hArg-, gluconoyl-Tyr-Arg-, Ac-diaminobutyryl-, Ac-diaminopropionyl-, N-propionyl-, N-butyryl-, N-valeryl-, N-methyl-Tyr-Arg-, N-glutaryl-Tyr-Arg-, N-succinyl-Tyr-Arg-, R⁶—SO₂NHC(O)CH₂CH₂C(O)—, R⁶—SO₂NHC(O)CH₂CH₂C(O)Arg-, R⁶—SO₂NHCH₂CH₂CH₂C(O)—, C₃-C₇ cycloalkylcarbonyl, phenylsulfonyl, C₈-C₁₄ bicyclic arylsulfonyl, phenyl-(CH₂)_{q}C(O)—, C₈-C₁₄ bicyclic aryl-(CH₂)_{q}C(O)—, 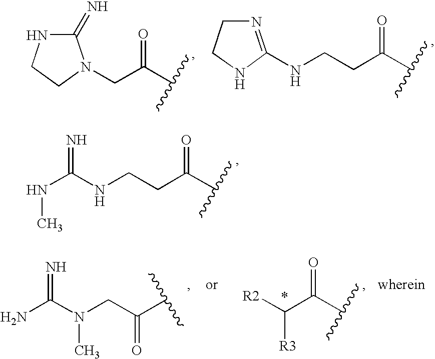
R² is —H, —NH₂, —NHC(O)CH₃, —NHC(O)(CH₂)₁₋₄CH₃, —NH-TyrC(O)CH₃, R⁶SO₂NH—, Ac-Cya-NH—, Tyr-NH—, HO—(C₆H₅)—CH₂CH₂C(O)NH—, or CH₃—(C₆H₅)—C(O)CH₂CH₂C(O)NH—;
R³ is C₁-C₄ straight or branched alkyl, NH₂—CH₂—(CH₂)_{q}—, HO—CH₂—, (CH₃)₂CHNH(CH₂)₄—, R⁶(CH₂)_{q}—, R⁶SO₂NH—, Ser, Ile, 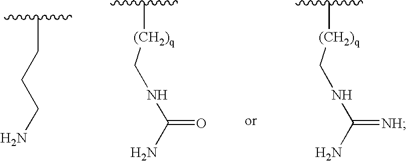
q is 0, 1, 2, or 3;
R⁶ is a phenyl or C₈-C₁₄ bicyclic aryl;
m is 1 or 2;
p is 1 or 2;
R⁴ is H, C₁-C₄ straight or branched alkyl, phenyl, benzyl, or (C₆H₅)—CH₂—O—CH₂—;
X is H, Cl, F, Br, methyl, or methoxy; and
R⁵ is —NH₂, —OH, glycinol, NH₂-Pro-Ser-, NH₂-Pro-Lys-, HO-Ser-, HO-Pro-Ser-, HO-Lys-, -Ser alcohol, -Ser-Pro alcohol, -Lys-Pro alcohol, HOCH₂CH₂—O—CH₂CH₂NH—, NH₂-Phe-Arg-, NH₂-Glu-, NH₂CH₂RCH₂NH—, RHN—, or RO— where R is a C₁-C₄ straight or branched alkyl.

In another preferred embodiment of the present invention are compounds of the Structural Formula III, wherein W is Glu or a single bond (viz., is absent); R₄ is H or CH₃; X is H, Cl, F, or Br; and R₅ is NH₂ or OH.

A preferred embodiment includes compounds of Structural Formula III wherein W is Glu or is absent; R¹ is H—, Ac—, Arg-, Ac-Arg-, or Ac-D-Arg-; m is 1 or 2; p is 1; and R⁵ is NH₂ or OH.

Another preferred embodiment of the invention includes a compound of Structural Formula III wherein W is absent; R¹ is Ac—; m is 2; p is 1; and R⁵ is NH₂.

Another preferred embodiment of the invention includes a compound of Structural Formula III wherein W is Glu; R¹ is Ac-Arg-; m is 1; p is 1; and R¹ is NH₂.

Another preferred embodiment of the invention includes a compound of Structural Formula III wherein W is absent; R¹ is H; m is 2; p is 1; and R⁵ is NH₂.

Another preferred embodiment of the invention includes a compound of Structural Formula III wherein W is absent; R¹ is Arg-; m is 2; p is 1; and R is OH.

A most preferred embodiment of the present invention includes a compound of Structural Formula III wherein W is Glu; R¹ is Ac-D-Arg-; m is 1; p is 1; and R⁵ is NH₂.

The present invention includes, but is not limited to, those compounds listed in the following table:

TABLE 1
Specific compounds within the present invention.

| No. | Name |
|---|---|
| 1 | Ac-cyclo[Cys-His-D-Phe-Arg-Trp-Cys]-NH₂ |
| 2 | Ac-Cya-Arg-cyclo[Cys-Ala-His-D-Phe-Arg-Trp-Cys]-NH₂ |
| 3 | Ac-Tyr-Arg-cyclo[Cys-Ala-His-D-Phe-Arg-Trp-Cys]-NH₂ |
| 4 | Ac-Tyr-Arg-cyclo[Cys-Arg-His-D-Phe-Arg-Trp-Cys]-NH₂ |
| 5 | Ac-Tyr-Arg-cyclo[Cys-Asn-His-D-Phe-Arg-Trp-Cys]-NH₂ |
| 6 | Ac-cyclo[Cys-Asp-His-D-Phe-Arg-Trp-Cys]-NH₂ |
| 7 | Ac-Tyr-Arg-cyclo[Cys-Asp-His-D-Phe-Arg-Trp-Cys]-NH₂ |
| 8 | Ac-cyclo[Cys-Gln-His-D-Phe-Arg-Trp-Cys]-NH₂ |
| 9 | Ac-Tyr-Arg-cyclo[Cys-Gln-His-D-Phe-Arg-Trp-Cys]-OH |
| 10 | Ac-Tyr-Arg-cyclo[Cys-Gln-His-D-Phe-Arg-Trp-Cys]-OMe |
| 11 | Tyr-Arg-cyclo[Cys-Gly-His-D-Phe-Arg-Trp-Cys]-NH₂ |
| 12 | Ac-Tyr-Arg-cyclo[Cys-Gly-His-D-Phe-Arg-Trp-Cys]-NH₂ |
| 13 | Ac-Tyr-Arg-cyclo[Cys-His-His-D-Phe-Arg-Trp-Cys]-NH₂ |
| 14 | Ac-Tyr-Arg-cyclo[Cys-Ile-His-D-Phe-Arg-Trp-Cys]-NH₂ |
| 15 | Ac-cyclo[Cys-Leu-His-D-Phe-Arg-Trp-Cys]-NH₂ |
| 16 | Ac-cyclo[Cys-Lys-His-D-Phe-Arg-Trp-Cys]-NH₂ |
| 17 | N-methyl-Tyr-Arg-cyclo[Cys-Met-His-D-Phe-Arg-Trp-Cys]-NH₂ |
| 18 | Ac-Tyr-Arg-cyclo[Cys-Met-His-D-Phe-Arg-Trp-Cys]-NH₂ |
| 19 | Ac-Tyr-Arg-cyclo[Cys-Phe-His-D-Phe-Arg-Trp-Cys]-NH₂ |
| 20 | Ac-Tyr-Arg-cyclo[Cys-Pro-His-D-Phe-Arg-Trp-Cys]-NH₂ |
| 21 | Ac-Tyr-Arg-cyclo[Cys-Ser-His-D-Phe-Arg-Trp-Cys]-NH₂ |
| 22 | Ac-Tyr-Arg-cyclo[Cys-Thr-His-D-Phe-Arg-Trp-Cys]-NH₂ |
| 23 | Ac-Tyr-Arg-cyclo[Cys-Trp-His-D-Phe-Arg-Trp-Cys]-NH₂ |
| 24 | Ac-Tyr-Arg-cyclo[Cys-Tyr-His-D-Phe-Arg-Trp-Cys]-NH₂ |
| 25 | Ac-Tyr-Arg-cyclo[Cys-Val-His-D-Phe-Arg-Trp-Cys]-NH₂ |
| 26 | Ac-Arg-cyclo[Cys-Cya-His-D-Phe-Arg-Trp-Cys]-NH₂ |
| 27 | Ac-D-Arg-cyclo[Cys-Cya-His-D-Phe-Arg-Trp-Cys]-NH₂ |
| 28 | Ac-Tyr-Arg-cyclo[Cys-Cya-His-D-Phe-Arg-Trp-Cys]-NH₂ |
| 29 | cyclo[Cys-Glu-His-D-Phe-Arg-Trp-Cys]-NH₂ |
| 30 | Ac-cyclo[Cys-Glu-His-D-Phe-Arg-Trp-Cys]-NH₂ |
| 31 | Ac-cyclo[Cys-Glu-His-(4-F-D-Phe)-Arg-Trp-Cys]-NH₂ |
| 32 | Ac-cyclo[Cys-Glu-His-(4-Cl-D-Phe)-Arg-Trp-Cys]-NH₂ |
| 33 | Ac-cyclo[Cys-Glu-His-(4-Br-D-Phe)-Arg-Trp-Cys]-NH₂ |
| 34 | Ac-cyclo[Cys-Glu-(1-Me-His)-D-Phe-Arg-Trp-Cys]-NH₂ |
| 35 | Ac-cyclo[Cys-Glu-His-D-Phe-Arg-Trp-Cys]-Lys-Pro-NH₂ |
| 36 | Ac-cyclo[Cys-Glu-His-D-Phe-Arg-Trp-Cys]-Ser-Pro-NH₂ |
| 37 | N-propionyl-cyclo[Cys-Glu-His-D-Phe-Arg-Trp-Cys]-NH₂ |
| 38 | N-butyryl-cyclo[Cys-Glu-His-D-Phe-Arg-Trp-Cys]-NH₂ |
| 39 | N-valeryl-cyclo[Cys-Glu-His-D-Phe-Arg-Trp-Cys]-NH₂ |
| 40 | 3-guanidinopropionyl-cyclo[Cys-Glu-His-D-Phe-Arg-Trp-Cys]-NH₂ |
| 41 | 4-guanidinobutyryl-cyclo[Cys-Glu-His-D-Phe-Arg-Trp-Cys]-NH₂ |
| 42 | 5-guanidinovaleryl-cyclo[Cys-Glu-His-D-Phe-Arg-Trp-Cys]-NH₂ |
| 43 | Ac-diaminopropionyl-cyclo[Cys-Glu-His-D-Phe-Arg-Trp-Cys]-NH₂ |
| 44 | Ac-diaminobutyryl-cyclo[Cys-Glu-His-D-Phe-Arg-Trp-Cys]-NH₂ |
| 45 | Arg-cyclo[Cys-Glu-His-D-Phe-Arg-Trp-Cys]-OH |
| 46 | D-Arg-cyclo[Cys-Glu-His-D-Phe-Arg-Trp-Cys]-NH₂ |
| 47 | Ac-D-Arg-cyclo[Cys-Glu-His-Phe-Arg-Trp-Cys]-NH₂ |
| 48 | Ac-Arg-cyclo[Cys-Glu-His-D-Phe-Arg-Trp-Cys]-NH₂ |
| 49 | Ac-Arg-cyclo[Cys-Glu-His-D-Phe-Arg-Trp-Cys]-OH |
| 50 | Ac-Arg-cyclo[Cys-Glu-His-(4-Cl-D-Phe)-Arg-Trp-Cys]-NH₂ |
| 51 | Ac-Arg-cyclo[Cys-Glu-(1-Me-His)-D-Phe-Arg-Trp-Cys]-NH₂ |
| 52 | Ac-D-Arg-cyclo[Cys-Glu-His-D-Phe-Arg-Trp-Cys]-NH₂ |
| 53 | Ac-D-Arg-cyclo[Cys-Glu-His-D-Phe-Arg-Trp-Cys]-OH |
| 54 | Ac-hArg-cyclo[Cys-Glu-His-D-Phe-Arg-Trp-Cys]-NH₂ |
| 55 | Ac-Cit-cyclo[Cys-Glu-His-D-Phe-Arg-Trp-Cys]-NH₂ |
| 56 | Ac-Cit-cyclo[Cys-Glu-(1-Me-His)-D-Phe-Arg-Trp-Cys]-NH₂ |
| 57 | Ac-Leu-cyclo[Cys-Glu-His-D-Phe-Arg-Trp-Cys]-NH₂ |
| 58 | Ac-Lys-cyclo[Cys-Glu-His-D-Phe-Arg-Trp-Cys]-NH₂ |
| 59 | Ac-Lys(ipr)-cyclo[Cys-Glu-His-D-Phe-Arg-Trp-Cys]-NH₂ |
| 60 | Ac-nLeu-cyclo[Cys-Glu-His-D-Phe-Arg-Trp-Cys]-NH₂ |
| 61 | Ac-nLeu-cyclo[Cys-Glu-His-D-Phe-Arg-Trp-Cys]-Ser-Pro-NH₂ |
| 62 | Ac-Orn-cyclo[Cys-Glu-His-D-Phe-Arg-Trp-Cys]-NH₂ |
| 63 | Ac-Val-cyclo[Cys-Glu-His-D-Phe-Arg-Trp-Cys]-NH₂ |
| 64 | N-(2-naphthalenesulfonyl)-D-Arg-cyclo[Cys-Glu-His-D-Phe-Arg-Trp-Cys]-NH₂ |
| 65 | N-(2-naphthalenesulfonylamino-4-oxo-butyryl)-D-Arg-cyclo[Cys-Glu-His-D-Phe-Arg-Trp-Cys]- |
| | NH₂ |
| 66 | 3-(4-hydroxyphenyl)propionyl-Arg-cyclo[Cys-Glu-His-D-Phe-Arg-Trp-Cys]-NH₂ |
| 67 | 3-(4-methylbenzoyl)propionyl-Arg-cyclo[Cys-Glu-His-D-Phe-Arg-Trp-Cys]-NH₂ |
| 68 | Tyr-Arg-cyclo[Cys-Glu-His-D-Phe-Arg-Trp-Cys]-NH₂ |
| 69 | Tyr-Arg-cyclo[Cys-Glu-His-D-Phe-Arg-Trp-Cys]-OH |
| 70 | Tyr-Arg-cyclo[Cys-Glu-His-D-Phe-Arg-Trp-Cys]-NH-(CH₂)₆-NH₂ |
| 71 | Tyr-Arg-cyclo[Cys-Glu-His-D-Phe-Arg-Trp-Cys]-Glu-NH₂ |
| 72 | Ac-Tyr-Arg-cyclo[Cys-Glu-His-D-Phe-Arg-Trp-Cys]-NH₂ |
| 73 | Ac-Tyr-Arg-cyclo[Cys-Glu-His-D-Phe-Arg-Trp-Cys]-OH |
| 74 | N-succinyl-Tyr-Arg-cyclo[Cys-Glu-His-D-Phe-Arg-Trp-Cys]-NH₂ |
| 75 | N-glutaryl-Tyr-Arg-cyclo[Cys-Glu-His-D-Phe-Arg-Trp-Cys]-NH₂ |
| 76 | N-glutaryl-Tyr-Arg-cyclo[Cys-Glu-His-D-Phe-Arg-Trp-Cys]-OH |
| 77 | gluconoyl-Tyr-Arg-cyclo[Cys-Glu-His-D-Phe-Arg-Trp-Cys]-NH₂ |
| 78 | Ac-Tyr-Arg-cyclo[Cys-Glu-His-D-Phe-Arg-Trp-Cys] alcohol |
| 79 | Ac-Tyr-D-Arg-cyclo[Cys-Glu-His-D-Phe-Arg-Trp-Cys]-NH₂ |
| 80 | Ac-Tyr-Arg-cyclo[D-Cys-Glu-His-D-Phe-Arg-Trp-Cys]-NH₂ |
| 81 | Ac-Tyr-Arg-cyclo[Cys-Glu-(1-Me-His)-D-Phe-Arg-Trp-Cys]-NH₂ |
| 82 | Ac-Tyr-Arg-cyclo[Cys-Glu-(1-Me-D-His)-D-Phe-Arg-Trp-Cys]-NH₂ |
| 83 | Ac-Tyr-Arg-cyclo[Cys-Glu-His-(4-F-D-Phe)-Arg-Trp-Cys]-NH₂ |
| 84 | Ac-Tyr-Arg-cyclo[Cys-Glu-(1-Me-His)-(4-F-D-Phe)-Arg-Trp-Cys]-NH₂ |
| 85 | Ac-Tyr-Arg-cyclo[Cys-Glu-(1-Me-D-His)-(4-F-D-Phe)-Arg-Trp-Cys]-NH₂ |
| 86 | Ac-Tyr-Arg-cyclo[Cys-Glu-His-(4-Cl-D-Phe)-Arg-Trp-Cys]-NH₂ |
| 87 | Ac-Arg-cyclo[Cys-Glu-(1-Me-His)-(4-Cl-D-Phe)-Arg-Trp-Cys]-NH₂ |
| 88 | Ac-Tyr-Arg-cyclo[Cys-Glu-(1-Me-D-His)-(4-Cl-D-Phe)-Arg-Trp-Cys]-NH₂ |
| 89 | Ac-Tyr-Arg-cyclo[Cys-Glu-His-(4-Br-D-Phe)-Arg-Trp-Cys]-NH₂ |
| 90 | Ac-Tyr-Arg-cyclo[Cys-Glu-(1-Me-His)-(4-Br-D-Phe)-Arg-Trp-Cys]-NH₂ |
| 91 | Ac-Tyr-Arg-cyclo[Cys-Glu-(1-Me-D-His)-(4-Br-D-Phe)-Arg-Trp-Cys]-NH₂ |
| 92 | Ac-Tyr-Arg-cyclo[Cys-Glu-His-(4-Me-D-Phe)-Arg-Trp-Cys]-NH₂ |
| 93 | Ac-Tyr-Arg-cyclo[Cys-Glu-His-(4-OMe-D-Phe)-Arg-Trp-Cys]-NH₂ |
| 94 | Ac-Tyr-Arg-cyclo[Cys-Glu-(1-Me-His)-(4-OMe-D-Phe)-Arg-Trp-Cys]-NH₂ |
| 95 | Ac-Tyr-Arg-cyclo[Cys-Glu-(1-Me-D-His)-(4-OMe-D-Phe)-Arg-Trp-Cys]-NH₂ |
| 96 | Ac-Tyr-Arg-cyclo[Cys-Glu-(3-Me-His)-D-Phe-Arg-Trp-Cys]-NH₂ |
| 97 | Ac-Tyr-Arg-cyclo[Cys-Glu-(5-Me-His)-D-Phe-Arg-Trp-Cys]-NH₂ |
| 98 | Ac-Tyr-Arg-cyclo[Cys-Glu-(5-Me-D-His)-D-Phe-Arg-Trp-Cys]-NH₂ |
| 99 | Ac-Tyr-Arg-cyclo[Cys-Glu-(1-benzyl-His)-D-Phe-Arg-Trp-Cys]-NH₂ |
| 100 | Ac-Tyr-Arg-cyclo[Cys-Glu-(1-benzyl-D-His)-D-Phe-Arg-Trp-Cys]-NH₂ |
| 101 | Ac-Tyr-Arg-cyclo[Cys-Glu-(1-Bom-His)-D-Phe-Arg-Trp-Cys]-NH₂ |
| 102 | Ac-Tyr-Arg-cyclo[Cys-Glu-(1-pyrazolyl-Ala)-D-Phe-Arg-Trp-Cys]-NH₂ |
| 103 | Ac-Tyr-Arg-cyclo[Cys-Glu-(4-phenyl-1H-imidazol-2-yl-Ala)-D-Phe-Arg-Trp-Cys]-NH₂ |
| 104 | Ac-Tyr-Arg-cyclo[Cys-Glu-(4-phenyl-1H-imidazol-2-yl-D-Ala)-D-Phe-Arg-Trp-Cys]-NH₂ |
| 105 | Ac-Tyr-Arg-cyclo[Cys-Glu-(2-pyrazine-Ala)-D-Phe-Arg-Trp-Cys]-NH₂ |
| 106 | Ac-Tyr-Arg-cyclo[Cys-Glu-(β-(1,2,4-triazol-3-yl))-Ala)-D-Phe-Arg-Trp-Cys]-NH₂ |
| 107 | Ac-Tyr-Arg-cyclo[Cys-Glu-(β-(1,2,4-triazol-3-yl))-D-Ala)-D-Phe-Arg-Trp-Cys]-NH₂ |
| 108 | Ac-Tyr-Arg-cyclo[Cys-Glu-(β-((1-benzyl)-1,2,4-triazol-3-yl))-Ala)-D-Phe-Arg-Trp-Cys]-NH₂ |
| 109 | Ac-Tyr-Arg-cyclo[Cys-Glu-(β-((1-benzyl)-1,2,4-triazol-3-yl))-D-Ala)-D-Phe-Arg-Trp-Cys]-NH₂ |
| 110 | Ac-Tyr-Arg-cyclo[Cys-Glu-(β-(2-furyl)-Ala)-D-Phe-Arg-Trp-Cys]-NH₂ |
| 111 | Ac-Tyr-Arg-cyclo[Cys-Glu-(β-(thien-2-yl)-Ala)-D-Phe-Arg-Trp-Cys]-NH₂ |
| 112 | Ac-Tyr-Arg-cyclo[Cys-Glu-(β-(1,3-thiazol-4-yl)-Ala)-D-Phe-Arg-Trp-Cys]-NH₂ |
| 113 | Ac-Tyr-Arg-cyclo[Cys-Glu-(β-(pyridin-4-yl)-Ala)-D-Phe-Arg-Trp-Cys]-NH₂ |
| 114 | Ac-Tyr-Arg-cyclo[Cys-Glu-His-D-Phe-Arg-Trp-Cys]-glycinol |
| 115 | Ac-Tyr-Arg-cyclo[Cys-Glu-His-D-Phe-Arg-Trp-Cys]-2-(2-aminoethoxy)ethanol |
| 116 | Ac-Tyr-Arg-cyclo[Cys-Glu-His-D-Phe-Arg-Trp-Cys]-Ser alcohol |
| 117 | Ac-Tyr-Arg-cyclo[Cys-Glu-His-D-Phe-Arg-Trp-Cys]-NH-(CH₂)₆-NH₂ |
| 118 | Ac-Tyr-Arg-cyclo[Cys-Glu-His-D-Phe-Arg-Trp-Cys]-Glu-NH₂ |
| 119 | Ac-Tyr-Arg-cyclo[Cys-Glu-His-D-Phe-Arg-Trp-Cys]-Ser-Pro-NH₂ |
| 120 | Ac-Tyr-Arg-cyclo[Cys-Glu-His-D-Phe-Arg-Trp-Cys]-Ser-Pro alcohol |
| 121 | Ac-Tyr-Arg-cyclo[Cys-Glu-His-D-Phe-Arg-Trp-Cys]-Lys-Pro-NH₂ |
| 122 | Ac-Tyr-Arg-cyclo[Cys-Glu-His-D-Phe-Arg-Trp-Cys]-Lys-Pro alcohol |
| 123 | Ac-Tyr-Arg-cyclo[Cys-Glu-His-D-Phe-Arg-Trp-Cys]-Arg-Phe-NH₂ |
| 124 | Ac-Tyr-Cit-cyclo[Cys-Glu-His-D-Phe-Arg-Trp-Cys]-NH₂ |
| 125 | Ac-Tyr-Cit-cyclo[Cys-Glu-(1-Me-His)-D-Phe-Arg-Trp-Cys]-NH₂ |
| 126 | Ac-Tyr-hArg-cyclo[Cys-Glu-His-D-Phe-Arg-Trp-Cys]-NH₂ |
| 127 | Ac-Tyr-(1-β-hArg)-cyclo[Cys-Glu-His-D-Phe-Arg-Trp-Cys]-NH₂ |
| 128 | Ac-Tyr-Lys-cyclo[Cys-Glu-His-D-Phe-Arg-Trp-Cys]-NH₂ |
| 129 | Ac-Tyr-Ser-cyclo[Cys-Glu-His-D-Phe-Arg-Trp-Cys]-NH₂ |
| 130 | Ac-Tyr-Val-cyclo[Cys-Glu-His-D-Phe-Arg-Trp-Cys]-NH₂ |
| 131 | N-succinyl-Tyr-Arg-cyclo[Cys-Glu-His-D-Phe-Arg-Trp-Cys]-OH |
| 132 | cyclo[hCys-His-D-Phe-Arg-Trp-Cys]-NH₂ |
| 133 | cyclo[hCys-His-D-Phe-Arg-Trp-Cys]-OH |
| 134 | cyclo[hCys-His-(4-F-D-Phe)-Arg-Trp-Cys]-NH₂ |
| 135 | cyclo[hCys-His-(4-Cl-D-Phe)-Arg-Trp-Cys]-NH₂ |
| 136 | Ac-cyclo[hCys-His-Phe-Arg-Trp-Cys]-NH₂ |
| 137 | Ac-cyclo[hCys-His-D-Phe-Arg-Trp-Cys]-NH₂ |
| 138 | Ac-cyclo[hCys-His-D-Phe-Arg-Trp-Cys]-OH |
| 139 | Ac-cyclo[hCys-His-(4-F-D-Phe)-Arg-Trp-Cys]-NH₂ |
| 140 | Ac-cyclo[hCys-His-(4-Cl-D-Phe)-Arg-Trp-Cys]-NH₂ |
| 141 | N-cyclopropanecarbonyl-cyclo[hCys-His-D-Phe-Arg-Trp-Cys]-NH₂ |
| 142 | N-cyclobutanecarbonyl-cyclo[hCys-His-D-Phe-Arg-Trp-Cys]-NH₂ |
| 143 | N-cyclopentanecarbonyl-cyclo[hCys-His-D-Phe-Arg-Trp-Cys]-NH₂ |
| 144 | N-cyclohexanecarbonyl-cyclo[hCys-His-D-Phe-Arg-Trp-Cys]-NH₂ |
| 145 | N-hexanoyl-cyclo[hCys-His-D-Phe-Arg-Trp-Cys]-NH₂ |
| 146 | N-benzoyl-cyclo[hCys-His-D-Phe-Arg-Trp-Cys]-NH₂ |
| 147 | 4-phenylbutyryl-cyclo[hCys-His-D-Phe-Arg-Trp-Cys]-NH₂ |
| 148 | 3-guanidinopropionyl-cyclo[hCys-His-D-Phe-Arg-Trp-Cys]-NH₂ |
| 149 | 5-guanidinovaleryl-cyclo[hCys-His-D-Phe-Arg-Trp-Cys]-NH₂ |
| 150 | N-phenylsulfonyl-cyclo[hCys-His-D-Phe-Arg-Trp-Cys]-NH₂ |
| 151 | N-(2-naphthalenesulfonyl)-cyclo[hCys-His-D-Phe-Arg-Trp-Cys]-NH₂ |
| 152 | N-(4-phenylsulfonamido-4-oxo-butyryl)-cyclo[hCys-His-D-Phe-Arg-Trp-Cys]-NH₂ |
| 153 | Arg-cyclo[hCys-His-D-Phe-Arg-Trp-Cys]-NH₂ |
| 154 | D-Arg-cyclo[hCys-His-D-Phe-Arg-Trp-Cys]-NH₂ |
| 155 | Arg-cyclo[hCys-His-D-Phe-Arg-Trp-Cys]-OH |
| 156 | Arg-cyclo[hCys-(1-Me-His)-D-Phe-Arg-Trp-Cys]-NH₂ |
| 157 | Arg-cyclo[hCys-(1-Me-D-His)-D-Phe-Arg-Trp-Cys]-NH₂ |
| 158 | Ac-Arg-cyclo[hCys-His-D-Phe-Arg-Trp-Cys]-NH₂ |
| 159 | Ac-Arg-cyclo[hCys-His-D-Phe-Arg-Trp-Cys]-OH |
| 160 | Ac-nLeu-cyclo[hCys-His-D-Phe-Arg-Trp-Cys]-NH₂ |
| 161 | phenylsulfonyl-Gly-cyclo[hCys-His-D-Phe-Arg-Trp-Cys]-NH₂ |
| 162 | Tyr-Arg-cyclo[hCys-His-D-Phe-Arg-Trp-Cys]-NH₂ |
| 163 | Tyr-Arg-cyclo[hCys-His-D-Phe-Arg-Trp-Cys]-OH |
| 164 | Ac-Tyr-Arg-cyclo[hCys-His-D-Phe-Arg-Trp-Cys]-NH₂ |
| 165 | Ac-Tyr-Arg-cyclo[hCys-His-D-Phe-Arg-Trp-Cys]-OH |
| 166 | Ac-Tyr-Arg-cyclo[hCys-Glu-His-D-Phe-Arg-Trp-Cys]-NH₂ |
| 167 | Ac-cyclo[hCys-His-(β-cyclohexyl-D-Ala)-Arg-Trp-Cys]-NH₂ |
| 168 | Ac-cyclo[hCys-His-D-Phe-Arg-Trp-penicillamine]-NH₂ |
| 169 | Ac-cyclo[hCys-His-(4-Cl-D-Phe)-Arg-Trp-penicillamine]-NH₂ |
| 170 | N-hexanoyl-cyclo[hCys-His-D-Phe-Arg-Trp-penicillamine]-NH₂ |
| 171 | N-cyclopentanecarbonyl-cyclo[hCys-His-D-Phe-Arg-Trp-penicillamine]-NH₂ |
| 172 | N-cyclohexanecarbonyl-cyclo[hCys-His-D-Phe-Arg-Trp-penicillamine]-NH₂ |
| 173 | N-benzoyl-cyclo[hCys-His-D-Phe-Arg-Trp-penicillamine]-NH₂ |
| 174 | 4-phenylbutyryl-cyclo[hCys-His-D-Phe-Arg-Trp-penicillamine]-NH₂ |
| 175 | N-phenylsulfonyl-cyclo[hCys-His-D-Phe-Arg-Trp-penicillamine]-NH₂ |
| 176 | (4-benzenesulfonamide)butyryl-cyclo[hCys-His-D-Phe-Arg-Trp-penicillamine]-NH₂ |
| 177 | Ac-nLeu-cyclo[hCys-His-D-Phe-Arg-Trp-penicillamine]-NH₂ |
| 178 | N-phenylsulfonyl-Gly-cyclo[hCys-His-D-Phe-Arg-Trp-penicillamine]-NH₂ |
| 179 | cyclo[3-thiopropionyl-His-D-Phe-Arg-Trp-hCys]-NH₂ |
| 180 | cyclo[Cys-His-D-Phe-Arg-Trp-hCys]-NH₂ |
| 181 | cyclo[Cys-His-(4-F-D-Phe)-Arg-Trp-hCys]-NH₂ |
| 182 | cyclo[Cys-His-(4-Cl-D-Phe)-Arg-Trp-hCys]-NH₂ |
| 183 | Ac-cyclo[Cys-His-D-Phe-Arg-Trp-hCys]-NH₂ |
| 184 | Ac-cyclo[Cys-His-(4-F-D-Phe)-Arg-Trp-hCys]-NH₂ |
| 185 | Ac-cyclo[Cys-His-(4-Cl-D-Phe)-Arg-Trp-hCys]-NH₂ |
| 186 | Arg-cyclo[Cys-His-D-Phe-Arg-Trp-hCys]-NH₂ |
| 187 | Arg-cyclo[Cys-His-(4-F-D-Phe)-Arg-Trp-hCys]-NH₂ |
| 188 | Arg-cyclo[Cys-His-(4-Cl-D-Phe)-Arg-Trp-hCys]-NH₂ |
| 189 | Ac-Arg-cyclo[Cys-His-D-Phe-Arg-Trp-hCys]-NH₂ |
| 190 | Ac-Arg-cyclo[Cys-His-(4-F-D-Phe)-Arg-Trp-hCys]-NH₂ |
| 191 | Ac-Arg-cyclo[Cys-His-(4-Cl-D-Phe)-Arg-Trp-hCys]-NH₂ |
| 192 | Ac-Tyr-Arg-cyclo[Cys-Glu-His-D-Phe-Arg-Trp-hCys]-NH₂ |
| 193 | Ac-cyclo[hCys-His-D-Phe-Arg-Trp-hCys]-NH₂ |
| 194 | Arg-cyclo[hCys-His-D-Phe-Arg-Trp-hCys]-NH₂ |
| 195 | Ac-Arg-cyclo[hCys-His-D-Phe-Arg-Trp-hCys]-NH₂ |
| 196 | Ac-Tyr-Arg-cyclo[hCys-His-D-Phe-Arg-Trp-hCys]-NH₂ |
| 197 | Ac-Tyr-Arg-cyclo[hCys-Glu-His-D-Phe-Arg-Trp-hCys]-NH₂ |
| 198 | Ac-cyclo(S-CH₂-S)[Cys-His-D-Phe-Arg-Trp-Cys]-NH₂ |

A preferred embodiment of the invention includes Compound Nos. 48, 52, 132, 137, and 155. More preferred is a group consisting of Compound Numbers 52 and 137. Another more preferred embodiment includes Compound Number 137, denoted by the name Ac-cyclo[hCys-His-D-Phe-Arg-Trp-Cys]-NH₂. A most preferred embodiment of the present invention includes Compound Number 52, denoted by the name Ac-D-Arg-cyclo[Cys-Glu-His-D-Phe-Arg-Trp-Cys]-NH₂.

In one embodiment, the present invention relates to pharmaceutical compositions comprising at least one compound of the present invention, or a pharmaceutically acceptable salt thereof, and a pharmaceutically acceptable carrier.

In another embodiment, the present invention relates to a method for agonizing the MC4 receptor, which comprises administering to a patient in need thereof an effective amount of a compound represented by Structural Formula I, Structural Formula II, or Structural Formula III, or a pharmaceutical salt thereof.

In another embodiment, the present invention relates to a method of treating obesity in a mammal, comprising the step of administering to the mammal in need thereof a pharmaceutically effective amount of at least one compound of Structural Formula I, Structural Formula II, or Structural Formula III, or a pharmaceutical salt thereof.

In another embodiment, the present invention relates to a method of treating diabetes mellitus in a mammal, comprising the step of administering to the mammal in need thereof a pharmaceutically effective amount of at least one compound of Structural Formula I, Structural Formula II, or Structural Formula III, or a pharmaceutical salt thereof.

In another embodiment, the present invention relates to a method of treating male and/or female sexual dysfunction in a mammal, comprising the step of administering to the mammal in need thereof a pharmaceutically effective amount of at least one compound of Structural Formula I, Structural Formula II, or Structural Formula III, or a pharmaceutical salt thereof.

In another embodiment, the present invention is further related to the use of the compound of Structural Formula I, Structural Formula II, or Structural Formula III, or a pharmaceutical salt thereof, as a medicament.

In another embodiment, the present invention is further related to the use of the compound of Structural Formula I, Structural Formula II, or Structural Formula III, or a pharmaceutical salt thereof, in the manufacture of a medicament for treating obesity.

In another embodiment, the present invention is further related to the use of the compound of Structural Formula I, Structural Formula II, or Structural Formula III, or a pharmaceutical salt thereof, in the manufacture of a medicament for treating diabetes mellitus.

In another embodiment, the present invention is further related to the use of the compound of Structural Formula I, Structural Formula II, or Structural Formula III, or a pharmaceutical salt thereof, in the manufacture of a medicament for treating sexual dysfunction.

The compounds of the present invention also can be effective in treating and preventing diabetes mellitus, and male and female sexual dysfunction. In addition, the compounds can be associated with a more favorable safety profile than compounds currently used to treat these conditions.

The terms used to describe the instant invention have the following meanings herein.

When a compound represented by Structural Formula I, Structural Formula II, or Structural Formula III has more than one chiral substituent, it may exist in diastereoisomeric forms. The diastereoisomeric pairs may be separated by methods known to those skilled in the art (for example, chromatography or crystallization), and the individual enantiomers within each pair may be separated using methods familiar to the skilled artisan. The present invention includes each diastereoisomer of compounds of Structural Formula I, Structural Formula II, and Structural Formula III, and mixtures thereof.

Certain compounds of Structural Formula I, Structural Formula II, and Structural Formula III may exist in different stable conformational forms, which may be separable. Torsional asymmetry due to restricted rotation about an asymmetric single bond, for example because of steric hindrance or ring strain, may permit separation of different conformers. The present invention includes each conformational isomer of compounds of Structural Formula I, Structural Formula II, and Structural Formula III, and mixtures thereof.

Certain compounds of Structural Formula I, Structural Formula II, and Structural Formula III may exist in zwitterionic form, and the present invention includes each zwitterionic form of compounds of Structural Formula I, Structural Formula II, or Structural Formula III, and mixtures thereof.

As used herein, “C₁-C₄ straight or branched alkyl” means a straight chained or branched hydrocarbon having 1 to 4 carbon atoms, which is completely saturated and unsubstituted. “C₃-C₇ cycloalkyl” refers to a saturated, unsubstituted hydrocarbon ring having 3 to 7 carbon atoms. A “C₁-C₄ straight or branched heteroalkyl” refers to a straight chained or branched hydrocarbon having 1 to 4 carbon atoms, which is completely saturated and unsubstituted, that also contains at least one “heteroatom.” A “heteroatom” is nitrogen, oxygen, or sulfur. “C₃-C₇ heterocycloalkyl” refers to a saturated, unsubstituted hydrocarbon ring having 3 to 7 carbon atoms, which also contains at least one “heteroatom.” C₁-C₄ straight or branched alkyl, C₃-C₇ cycloalkyl, C₁-C₄ straight or branched heteroalkyl, and C₃-C₇ heterocycloalkyl may be used as generic modifiers to describe a genus of substituents on another functional group such as a carbonyl, sulfonyl, or sulfonamide. For example, a “C₃-C₇ cycloalkylcarbonyl” refers to a genus of saturated, unsubstituted hydrocarbon rings having 3 to 7 carbon atoms that are bonded to a carbonyl group.

A “C₈-C₁₄ bicyclic aryl” refers to two or three hydrocarbon rings fused together, having 8 to 14 carbon atoms, such as naphthalene. A C₈-C₁₄ bicyclic aryl ring system has at least one aromatic ring. A “5- or 6-membered heteroaryl” refers to a monocyclic aromatic ring having 5 or 6 atoms, of which 1-4 atoms are heteroatoms. An “8- to 14-membered bicyclic heteroaryl” ring refers to two or three hydrocarbon rings fused together, having 8 to 14 atoms, at least one aromatic ring, and 1-4 heteroatoms.

A phenyl, benzyl, benzoyl, C₈-C₁₄ bicyclic aryl, 5- or 6-membered heteroaryl, or 8- to 14-membered bicyclic heteroaryl may be unsubstituted or substituted with C₁-C₄ straight or branched alkyl, F, Cl, Br, —OH, methoxy, phenyl, benzyl, benzoyl, or benzyloxymethyl. Furthermore, phenyl, benzyl, benzoyl, C₈-C₁₄ bicyclic aryl, 5- or 6-membered heteroaryl, and 8- to 14-membered bicyclic heteroaryl may be used as generic modifiers to describe a genus of substituents on another functional group such as a carbonyl, sulfonyl, or sulfonamide. For example, a “C₈-C₁₄ bicyclic arylsulfonyl” refers to a genus of bicyclic aryl rings having 8 to 14 carbon atoms that are bonded to a sulfonyl group.

Modified amino acids are indicated by parentheses around the amino acid and the modification thereto (e.g., (4-Cl-D-Phe) is a 4-chloro modification on the D-isomer of phenylalanine). With respect to moieties depicted in Structural Formula I, Structural Formula II, and Structural Formula III, the single letter designations are as defined and do not refer to single letter amino acids corresponding to those letters.

The letter “D” preceding the above-mentioned 3-letter abbreviations, e.g., “D-Phe,” means the D-form of the amino acid. When the single letter abbreviation is used for an amino acid, a “d” will precede the letter to designate the D-form of the amino acid (e.g., dF=D-Phe).

An “amino alcohol” is an amino acid that has been modified by reducing the carbonyl group of the C-terminus to a methylene group. Amino alcohols are denoted by the general nomenclature “Xaa alcohol,” wherein Xaa is the specific amino acid, from which the carbonyl group has been removed. To illustrate, “Ser alcohol” has the structure H₂N—CH(CH₂OH)—CH₂OH as opposed to the Ser amino acid structure of H₂N—CH(CH₂OH)—COOH.

“Single bond,” as used herein, refers to a structure that does not contain an amino acid at the specified position. It is used to signify that an amino acid is absent from that position such that the carbonyl adjacent to that position on one side and the amine adjacent to that position on the other side form a peptide bond with each other.

“*” means that both the D- and L-isomers are possible.

“Ac” refers to acetyl (i.e., —C(O)CH₃).

“Orn” refers to omithine.

“hCys” refers to homocysteine.

“hArg” refers to homoarginine.

“Lys(ipr)” refers to lysine(N-isopropyl).

“Cit” refers to citrulline.

“nLeu” refers to norleucine.

“Me” refers to methyl.

“OMe” refers to methoxy.

“Cya” refers to cysteic acid.

“Dap” refers to diaminopropionyl.

“Dab” refers to diaminobutyryl.

“MC4 agonist” refers to a compound that has affinity for the MC4 receptor and results in measurable biological activity in cells, tissues, and organisms containing the MC4 receptor. Assays measuring such activity are well known in the art.

The term “selective” means having an activation preference for a certain receptor over other receptors which can be quantified based on whole cell, tissue, or organism assays which demonstrate receptor activity. Selectivity is ascertained by comparison of EC₅₀ values at the relevant receptors referenced.

“Pharmaceutically-acceptable salt” refers to salts of the compounds of the Structural Formula I, Structural Formula II, or Structural Formula III that are substantially non-toxic to mammals. Typical pharmaceutically acceptable salts include those salts prepared by reaction of the compounds of the present invention with a mineral or organic acid or an organic or inorganic base. Such salts are known as acid addition and base addition salts, respectively. It should be recognized that the particular counterion forming a part of any salt of this invention is not of a critical nature, so long as the salt as a whole is pharmaceutically acceptable and as long as the counterion does not contribute undesired qualities to the salt as a whole.

A pharmaceutical “acid addition salt” is a salt formed by reaction of the free base form of a compound of formula I with a pharmaceutical acid, such as described in the Encyclopedia of Pharmaceutical Technology, editors James Swarbrick and James C. Boylan, Vol. 13 (1996), “Preservation of Pharmaceutical Products to Salt Forms of Drugs and Absorption.” Specific salt forms include, but are not limited to the: acetate, benzoate, benzenesulfonate, 4-chlorobenzenesulfonate; citrate; ethanesulfonate; fumarate; d-gluconate; d-glucuronate; glutarate; glycolate; hippurate; hydrochloride; 2-hydroxyethanesulfonate; dl-lactate; maleate; d-malate; l-malate; malonate; d-mandelate; l-mandelate; methanesulfonate; 1,5-napthalenedisulfonate; 2-naphthalenesulfonate; phosphate; salicylate; succinate; sulfate; d-tartrate; l-tartrate; and p-toluenesulfonate.

A pharmaceutical “base addition” salt is a salt formed by reaction of the free acid form of a compound of formula I with a pharmaceutical base, such as described in the Encyclopedia of Pharmaceutical Technology, supra. Specific salt forms include, but are not limited to the: calcium, diethanolamine, diethylamine, ethylenediamine, lysine, magnesium, piperazine, potassium, sodium, and tromethamine (Tris, Trizma) salts.

The term “active ingredient” means the compounds generically described by Structural Formula I, Structural Formula II, or Structural Formula III, as well as the salts of such compounds.

The term “pharmaceutically acceptable” means that the carrier, diluent, excipients, and salt must be compatible with the other ingredients of the composition and not clinically deleterious to the recipient thereof. Pharmaceutical compositions of the present invention are prepared by procedures known in the art using well-known and readily available ingredients.

The terms “treating” and “treat”, as used herein, include their generally accepted meanings, i.e., alleviating, ameliorating, managing, preventing, prohibiting, restraining, slowing, stopping, or reversing the progression or severity of a pathological condition, or sequela thereof, described herein.

The diseases, disorders or conditions for which compounds of the present invention are useful in treating include (1) obesity, (2) diabetes mellitus, and (3) male and/or female sexual dysfunction.

“Preventing” refers to reducing the likelihood that the recipient will incur or develop any of the pathological conditions described herein. The term “preventing” is particularly applicable to a patient that is susceptible to the particular pathological condition as determined by medical diagnosis.

“Pharmaceutically effective amount” means that amount of a compound, or salt thereof, that will elicit the biological or medical response of a tissue, system, or mammal and/or is capable of treating the conditions described herein, or that is capable of agonizing the MC3 and/or MC4 receptors. An “effective amount” of the peptide administered to a subject will also depend on the type and severity of the disease or condition and on the characteristics of the subject, such as general health, age, sex, body weight and tolerance to drugs. The recipient patient's physician should determine the therapeutic dose administered in light of the relevant circumstances.

A pharmaceutically effective amount can be administered prophylactically to a patient thought to be susceptible to development of a disease or condition. Such amount, when administered prophylactically to a patient, can also be effective to prevent or lessen the severity of the mediated condition. The dosage regimen utilizing the compounds of the present invention is selected by one of ordinary skill in the medical or veterinary arts, in view of a variety of factors, including, without limitation, the route of administration, the prior medical history of the recipient, the pathological condition or symptom being treated, the severity of the condition/symptom being treated, and the age and sex of the recipient patient. However, it will be understood that the therapeutic dose administered will be determined by the attending physician in the light of the relevant circumstances.

Generally, an effective minimum daily dose of a compound of the present invention will exceed about 0.01 mg. Typically, an effective maximum daily dose will not exceed about 1000 mg. More preferably, an effective minimum daily dose will be between about 0.05 mg and 50 mg, more preferably between. 0.1 mg and 10 mg. Most preferably, an effective minimum daily dose of an MC4R agonist peptide in the present invention will exceed about 2 μg/kg and will not exceed about 20 μg/kg. The exact dose may be determined, in accordance with the standard practice in the medical arts of “dose titrating” the recipient; that is, initially administering a low dose of the compound, and gradually increasing the does until the desired therapeutic effect is observed. The desired dose may be presented in a single dose or as divided doses administered at appropriate intervals.

A “mammal” is an individual animal that is a member of the taxonomic class Mammalia. The class Mammalia includes humans, monkeys, chimpanzees, gorillas, cattle, swine, horses, sheep, dogs, cats, mice, and rats. The attending physician of ordinary skill can identify humans who will benefit from administration of the compounds and compositions of the present invention.

The term “patient” includes human and non-human animals such as companion animals (dogs and cats and the like), farm animals, and laboratory animals.

The term “pharmaceutical” when used herein as an adjective means substantially non-deleterious to the recipient patient.

A pharmaceutically effective amount of a compound of Structural Formula I, Structural Formula II, or Structural Formula III can be used for the preparation of a medicament useful for treating weight loss, obesity, diabetes and male and female sexual dysfunction.

Formulation:

The present pharmaceutical compositions are prepared by known procedures using well-known and readily available ingredients. Such procedures may include, e.g., conventional mixing, dissolving, granulating, dragee-making, levigating, emulsifying, encapsulating, entrapping or lyophilizing processes.

Because compounds of the invention contain an acidic moiety (i.e., carboxy), the compounds of the invention may be formulated as a pharmaceutical base addition salt thereof, e.g., as the sodium salt. Similarly, because compounds of the invention contain a basic moiety (i.e., amino), the compounds can be formulated as a pharmaceutical acid addition salt, e.g., as the acetate salt.

In making the compositions of the present invention, the active ingredient (a compound of the present invention) will usually be mixed with a carrier, or diluted by a carrier, or enclosed within a carrier. When the carrier serves as a diluent, it may be a solid, semisolid, or liquid material that acts as a vehicle, excipient, or medium for the active ingredient. Thus, the compositions can be in the form of, e.g., a suspension, solution, or sterile injectable solution.

An injectable formulation, for example, a sterile injectable aqueous or oleaginous suspension, can be prepared using suitable dispersing or wetting agents and suspending agents. The sterile injectable formulation may be a solution or suspension in a nontoxic parenterally acceptable diluent or solvent, for example, as a solution in 1,3-butanediol. Among the acceptable vehicles and solvents that may be employed are water, sterile water for injection (WFI), bacteriostatic water for injection (BWFI), Ringer's solution, and isotonic sodium chloride solution. In addition, sterile fixed oils are conventionally employed as a solvent or suspending medium. Fixed oils and fatty acids, such as oleic acid, may be employed in the preparation of an injectable formulation.

The compounds of the present invention, and the pharmaceutically acceptable salts, have valuable pharmacological properties and can be used in pharmaceutical compositions containing a pharmaceutically effective amount of a compound of the present invention, or pharmaceutically acceptable salts thereof, in combination with one or more pharmaceutically acceptable excipients. Excipients may include substances such as carriers, diluents, fillers, flavoring agents, sweeteners, lubricants, solubilizers, suspending agents, wetting agents, binders, disintegrating agents, encapsulating material, antimicrobial agents, and other conventional adjuvants. Proper formulation is dependent upon the route of administration chosen as well as any interactions between excipients. Pharmaceutical compositions typically contain from about 1 to about 99 weight percent of the active ingredient, which is a compound of the present invention.

Solid form formulations may include powders, tablets, and capsules. A solid carrier can be one or more substance that may also act as flavoring agents, lubricants, solubilizers, suspending agents, binders, tablet disintegrating agents, and encapsulating material.

Sterile liquid formulations may include suspensions, emulsions, syrups, and elixirs. The active ingredient may be dissolved or suspended in a pharmaceutically acceptable carrier, such as sterile water, sterile organic solvent, or a mixture of both sterile water and sterile organic solvent. The injectable formulation may be sterilized, for example, by filtration through a bacteria- or virus-retaining filter, by radiation, or by incorporating sterilizing agents in the form of sterile solid compositions which can be dissolved or dispersed in sterile water or other sterile injectable media just prior to use.

The compounds of the present invention may be formulated in a unit dosage form prior to administration to the recipient patient. A “unit dosage form” is a physically discrete unit containing a unit dose, suitable for administration in human subjects or other mammals. For example, a unit dosage form can be a capsule or tablet, or a number of capsules or tablets. A “unit dose” is a predetermined quantity of the active compound of the present invention, calculated to produce the desired therapeutic effect, generally in association with one or more pharmaceutically acceptable excipients. The quantity of active ingredient in a unit dose may be varied or adjusted from about 0.01 to about 1000 milligrams according to the particular treatment involved.

The compounds of the present invention can be administered in a single daily dose, or the total daily dose may be administered in divided doses, two, three, or more times per day, or by continuous infusion. Where delivery is via transdermal forms, of course, administration is continuous.

The compounds of the present invention can be administered by a variety of routes, including the oral, subcutaneous, topical, parenteral (e.g., intravenous and intramuscular), bronchial, or intranasal routes.

“Continuous infusion” of a compound of the present invention refers to controlled parenteral delivery of the peptide to a patient for an extended period of time. Administration via continuous infusion may be accomplished by, but is not limited to, delivery via pump, depot, suppository, pessary, transdermal patch or other topical administration (such as buccal, sublingual, spray, ointment, creme, or gel) using, for example, subcutaneous, intramuscular, intraperitoneal, intravenous, intracerebral, or intraarterial administration.

A pump delivering a compound of the present invention into the body may be implanted in the patient's body. Alternatively, the patient may wear a pump externally, being attached to the patient's body via catheter, needle, or some other connective means. Any pump that is suitable for the delivery of pharmaceuticals to a patient may be used. Examples include pumps such as those disclosed in U.S. Pat. No. 6,659,982.

A depot is a biocompatible polymer system containing a compound of the present invention and delivering the peptide over time. Examples include microspheres, microcapsules, nanoparticles, liposomes, a hydrogel, or other polymeric implants. Preferred periods for delivery of agonist by depot include one week, two weeks, and one month periods. If needed, another depot will be delivered to the patient for continued delivery of peptide.

Engineering a compound of the present invention to have a prolonged half-life will also result in continuous delivery of the MC4 receptor agonist to the receptor. Such modifications include conjugations with larger proteins such as albumin, antibody and antigen or chemical modifications that may increase half-life by linking fatty acids, polyethylene glycol (PEG) polymers, and other agents.

The compounds of the instant invention may be used effectively alone or in combination with one or more additional active agents depending on the desired target therapy. Combination therapy includes administration of a single pharmaceutical dosage composition which contains a compound of Structural Formula I, Structural Formula II, or Structural Formula III, and one or more additional active agents, as well as administration of a compound of Structural Formula I, Structural Formula II, or Structural Formula III, and each active agent in its own separate pharmaceutical dosage formulation. Where separate dosage formulations are used, a compound of Structural Formula I, Structural Formula II, or Structural Formula III, and one or more additional active agents can be administered at essentially the same time, i.e., concurrently, or at separately staggered times, i.e., sequentially; combination therapy is understood to include all of these regimens.

A preferred combination therapy for the treatment of obesity is the use of a compound of the present invention in combination with sibutramine (or active metabolites of sibutramine, e.g., desmethyl sibutramine and di-desmethyl sibutramine), preferably with sibutramine hydrochloride monohydrate. Another preferred combination is the use of a compound of the present invention in combination with orlistat.

A preferred combination therapy for the treatment of sexual dysfunction (erectile dysfunction) is the use of a compound of the present invention in combination with sildenafil citrate. Another preferred combination is the use of a compound of the present invention in combination with tadalafil. Yet another preferred combination is the use of a compound of the present invention in combination with vardenafil, preferably vardenafil hydrochloride.

The following examples are not intended to limit the invention in any way. All peptides of the present invention can be synthesized by solid-phase synthesis methods (Merrifield, J. Am. Chem. Soc. 85:2149-54, 1963) either by manual or automated synthesis techniques. The automated assembly can be carried out using either as ABI 431A or 433A synthesizer.

### EXAMPLE 1

### Synthesis of Compound No. 48: Ac-Arg-cyclo[Cys-Glu-His-D-Phe-Arg-Trp-Cys]-NH₂

The sequence Arg-Cys-Glu-His-D-Phe-Arg-Trp-Cys is assembled by standard Fmoc chemistry utilizing an ABI 431 instrument, according to Scheme A outlined below. The automated assembly is carried out by using the standard Applied Biosystems single 1.5 hour dicyclohexylcarbodiimide/hydroxybenzotriazole (DCC/HOBt) activation protocol. The solid support utilized is Rink MBHA resin (Rink, Tet. Lett. 28:3787-90, 1987) and the side chain protecting group scheme is: Arg(Pbf), Cys(Trt), Qlu(OtBu), Gln(Trt), His(Trt), Trp(Boc), Tyr(tBu). The protected amino acids and Rink resin can be purchased from Nova Biochem or Midwest Biotech. Acetylation of the cc-amino group, after the chain assembly, is carried out off-line with 5 equivalents acetic anhydride, 10 equivalents DIEA in dry DMF or NMP, 1 h at room temperature. The finished peptide is simultaneously deprotected and cleaved from the resin using a scavenger cocktail of TFA/H₂O/TIS/EDT (95/2/1/2, v/v), or TFA/H₂O/TIS/anisole (92/2/4/2, v/v) 2 hours at room temperature. The solvents are then evaporated under vacuum, and the peptide is precipitated and washed three times with cold diethyl ether to remove the scavengers. The crude product is used directly in the cyclization reaction.

Cyclization Protocol

The oxidation of the free cysteine sulfhydryl groups is accomplished by either air oxidation in 0.2 M ammonium acetate buffer containing 20% dimethyl sulfoxide (DMSO) at pH 7.0, or by treatment with 2,2′-pyridyldisulfide in 2.7 M guanidine buffer containing 30% DMSO. In each case, the final product is isolated by high performance liquid chromatography.

Purification

Purification is accomplished using standard preparative HPLC techniques. Immediately following the cyclization, the peptide is diluted and loaded onto an HPLC column and eluted with an aqueous 0.1% trifluoroacetic acid/acetonitrile gradient while monitoring at 214 nm. The appropriate fractions are pooled and lyophilized. Further characterization of the final product is performed using analytical HPLC and mass spectral analysis known in the art, and the data are summarized in Table 2 below.

Conversion to Acetate Salt

The peptide is adsorbed onto a 2.1×25 cm Zorbax C18 preparative column, which is equilibrated with 0.1% TFA/H₂O. The column is then washed with 2 volumes of 0.1 M ammonium acetate/5% acetonitrile followed by 2 column volumes of water. The peptide is eluted using 2% acetic acid and lyophilized.

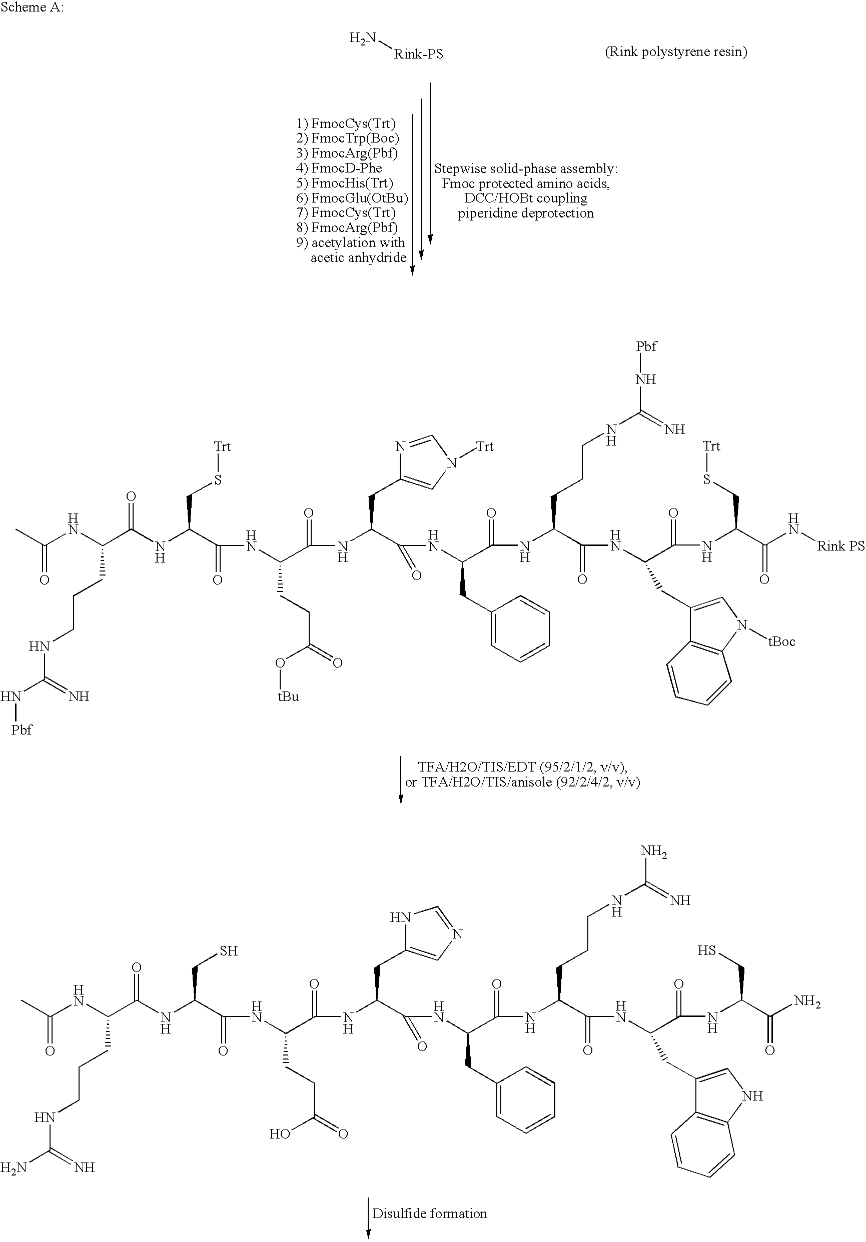

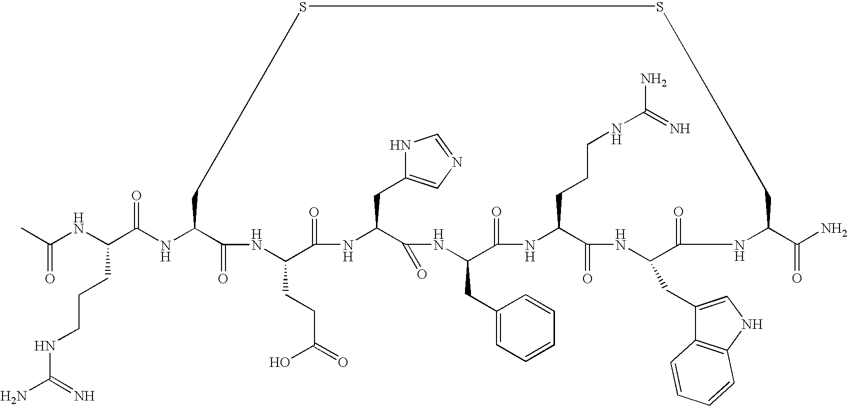

The following compounds are exemplified only for the purpose of illustration and should not be considered to limit the invention in any way.

### EXAMPLE 2

### Synthesis of Compound No. 1: Ac-cyclo[CVS-His-D-Phe-Arg-Trp-CYS]-NH₂

Can be prepared according to Example 1, with the exception that Emoc-Glu(OtBu) and Fmoc-Arg(pbf) in steps 6 and 8, respectively, are not used.

### EXAMPLE 3

### Synthesis of Compound No. 2: Ac-Cya-Arg-cyclo[Cys-Ala-Ris-D-Phe-Arg-Trp-Cys]-NH₂

Can be prepared according to Example 1, with the exception that Fmoc-Glu(OtBu) in step 6 is replaced with Fmoc-Ala. Between steps 8 and 9, oneyextra step of Fmoc-Cya (Fmoc-cysteic acid) is added. In addition, peptide cyclization (forming the disulfide bond) is carried out on resin using 10 equivalents of iodine in DMF for 2 h at room temperature.

### EXAMPLE 4

### Synthesis of Compound No. 3: Ac-Tyr-Arg-cyclo[Cys-Ala-His-D-Phe-Arg-Trp-Cys]-NH₂

Can be prepared according to Example 1, with the exception that Fmoc-Ala is used instead of Fmoc-Glu(OtBu) in step 6. Fmoc-Tyr(tBu) is added between steps 8 and 9.

### EXAMPLE 5

### Synthesis of Compound No. 4: Ac-Tyr-Arg-cyclo[Cys-Arg-His-D-Phe-Arg-Trp-Cys]-NH₂

Can be prepared according to Example 1, with the exception that Fmoc-Arg(Pbf) is used instead of Fmoc-Glu(OtBu) in step 6. Fmoc-Tyr(tBu) is used between steps 8 and 9.

### EXAMPLE 6

### Synthesis of Compound No. 5: Ac-Tyr-Arg-cyclo[Cys-Asn-His-D-Phe-Arg-Trp-Cys]-NH₂

Can be prepared according to Example 1, with the exception that Fmoc-Asn is used instead of Fmoc-Glu(OtBu) in step 6. Fmoc-Tyr(tBu) is added between steps 8 and 9.

### EXAMPLE 7

### Synthesis of Compound No. 6: Ac-cyclo[Cys-Asp-His-D-Phe-Arg-Trp-Cys]-NH₂

Can be prepared according to Example 1, with the exception that Fmoc-Arg(Pbf) is not used in step 8. Fmoc-Asp is used instead of Fmoc-Glu(OtBu) in step 6.

### EXAMPLE 8

### Synthesis of Compound No. 7: Ac-Tyr-Arg-cyclo[Cys-Asp-His-D-Phe-Arg-Trp-Cys]-NH₂

Can be prepared according to Example 1, with the exception that Fmoc-Asp is used instead of Fmoc-Glu(OtBu) in step 6. Fmoc-Tyr(tBu) is added between steps 8 and 9.

### EXAMPLE 9

### Synthesis of Compound No. 8: Ac-cyclo[Cys-Gln-His-D-Phe-Arg-Trp-Cys]-NH₂

Can be prepared according to Example 1, with the exception that Fmoc-Arg(Pbf) is not used in step 8. Fmoc-Gln is used instead of Fmoc-Glu(OtBu) in step 6.

### EXAMPLE 10

### Synthesis of Compound No. 9: Ac-Tyr-Arg-cyclo[Cys-Gln-His-D-Phe-Arg-Trp-Cys]-OH

Can be prepared according to Example 1, with the exception that: Step 1 Fmoc-Cys(Trt) is not used; Fmoc-Glu(Trt) is used instead of Fmoc-Glu(OtBu) in step 6. In addition, preloaded Fmoc-Cys(Trt)-Wang resin (Wang, J. Am. Chem. Soc. 95:1328-33, 1972) is used instead of Rink resin.

### EXAMPLE 11

### Synthesis of Compound No. 10: Ac-Tyr-Arg-cyclo[Cys-Glu-His-D-Phe-Arg-Trp-Cys]-OMe

Can be prepared according to Example 10. After the cleavage, cyclization, and purification, the peptide (Compound No. 9) is dissolved in dry methanol. Then, hydrochloride gas is bubbled into the methanol solution for about half minute. The reaction is allowed to proceed at room temperature for ten minutes. The solvents are removed under vacuum, and the final product is purified as specified in Example 1.

### EXAMPLE 12

### Synthesis of Compound No. 11: Tyr-Arg-cyclo[Cys-Gly-His-D-Phe-Arg-TrM-Cys]-NH₂

Can be prepared according to Example 1, with the exception that Fmoc-Gly is used instead of Fmoc-Glu(OtBu) in step 6. Fmoc-Tyr(tBu) is added after step 8. Acetylation with acetic anhydride in step 9 is omitted.

### EXAMPLE 13

### Synthesis of Compound No. 12: Ac-Tyr-Arg-cyclo[Cys-Gly-His-D-Phe-Arg-Trp-Cys]-NH₂

Can be prepared according to Example 1, with the exception that Fmoc-Gly is used instead of Fmoc-Glu(OtBu) in step 6. Fmoc-Tyr(tBu) is used between steps 8 and 9.

### EXAMPLE 14

### Synthesis of Compound No. 13: Ac-Tyr-Arg-cyclo[Cys-His-His-D-Phe-Arg-Trp-Cys]-NH₂

Can be prepared according to Example 1, with the exception that Fmoc-His is used instead of Fmoc-Glu(OtBu) in step 6. Fmoc-Tyr(tBu) is used between steps 8 and 9.

### EXAMPLE 15

### Synthesis of Compound No. 14: Ac-Tyr-Arg-cyclo[Cys-Ile-His-D-Phe-Arg-Trp-Cys]-NH₂

Can be prepared according to Example 1, with the exception that Fmoc-Ile is used instead of Fmoc-Glu(OtBu) in step 6. Fmoc-Tyr(tBu) is used between steps 8 and 9.

### EXAMPLE 16

### Synthesis of Compound No. 15: Ac-cyclo[Cys-Leu-His-D-Phe-Arg-Trp-Cys]-NH₂

Can be prepared according to Example 1, with the exception that Fmoc-Arg(Pbf) is not used in step 8. Fmoc-Leu is used instead of Fmoc-Glu(OtBu) in step 6.

### EXAMPLE 17

### Synthesis of Compound No. 16: Ac-cyclo[Cys-Lys-His-D-Phe-Arg-Trp-Cys]-NH₂

Can be prepared according to Example 1, with the exception that Fmoc-Arg(Pbf) is not used in step 8. Fmoc-Lys(Boc) is used instead of Fmoc-Glu(OtBu) in step 6.

### EXAMPLE 18

### Synthesis of Compound No. 17: N-methyl-Tyr-Arg-cyclo[Cys-Met-His-D-Phe-Arg-Trp-Cys]-NH₂

Can be prepared according to Example 1, with the exception that acetylation with acetic anhydride in step 9 is not used. Fmoc-N-methyl-Tyr is used after step 8. In addition, Fmoc-Met is used instead of Fmoc-Glu(OtBu) in step 6.

### EXAMPLE 19

### Synthesis of Compound No. 18: Ac-Tyr-Arg-cyclo[Cys-Met-His-D-Phe-Arg-Trp-Cys]-NH₂

Can be prepared according to Example 1, with the exception that Fmoc-Met is used instead of Fmoc-Glu(OtBu) in step 6. Fmoc-Tyr(tBu) is used between steps 8 and 9.

### EXAMPLE 20

### Synthesis of Compound No. 19: Ac-Tyr-Arg-cyclo[Cys-Phe-His-D-Phe-Are-Trp-Cys]-NH₂

Can be prepared according to Example 1, with the exception that Fmoc-Phe is used instead of Fmoc-Glu(OtBu) in step 6. Fmoc-Tyr(tBu) is used between steps 8 and 9.

### EXAMPLE 21

### Synthesis of Compound No. 20: Ac-Tyr-Arg-cyclo[Cys-Pro-His-D-Phe-Arg-Trp-Cys]-NH₂

Can be prepared according to Example 1, with the exception that Fmoc-Pro is used instead of Fmoc-Glu(OtBu) in step 6. Fmoc-Tyr(tBu) is used between steps 8 and 9.

### EXAMPLE 22

### Synthesis of Compound No. 21: Ac-Tyr-Arg-cyclo[Cys-Ser-His-D-Phe-Arg-Trp-Cys]-NH₂

Can be prepared according to Example 1, with the exception that Fmoc-Ser is used instead of Fmoc-Glu(OtBu) in step 6. Fmoc-Tyr(tBu) is used between steps 8 and 9.

### EXAMPLE 23

### Synthesis of Compound No. 22: Ac-Tyr-Arg-cyclo[Cys-Thr-His-D-Phe-Arg-Trp-Cys]-NH₂

Can be prepared according to Example 1, with the exception that Fmoc-Thr is used instead of Fmoc-Glu(OtBu) in step 6. Fmoc-Tyr(tBu) is used between steps 8 and 9.

### EXAMPLE 24

### Synthesis of Compound No. 23: Ac-Tyr-Arg-cyclo[Cys-Trp-His-D-Phe-Arg-Trp-Cys]-NH₂

Can be prepared according to Example 1, with the exception that Fmoc-Trp is used instead of Fmoc-Glu(OtBu) in step 6. Fmoc-Tyr(tBu) is used between steps 8 and 9.

### EXAMPLE 25

### Synthesis of Compound No. 24: Ac-Tyr-Arg-cyclo[Cys-Tyr-His-D-Phe-Arg-Trp-Cys]-NH₂

Can be prepared according to Example 1, with the exception that Fmoc-Tyr(tBu) is used instead of Fmoc-Glu(OtBu) in step 6. Fmoc-Tyr(tBu) is added between steps 8 and 9.

### EXAMPLE 26

### Synthesis of Compound No. 25: Ac-Tyr-Arg-cyclo[Cys-Val-His-D-Phe-Arg-Trp-Cys]-NH₂

Can be prepared according to Example 1, with the exception that Fmoc-Val is used instead of Fmoc-Glu(OtBu) in step 6. Fmoc-Tyr(tBu) is used between steps 8 and 9.

### EXAMPLE 27

### Synthesis of Compound No. 26: Ac-Arg-cyclo[Cys-Cya-His-D-Phe-Arg-Trp-Cys]-NH₂

Can be prepared according to Example 1, with the exception that Fmoc-Glu(OtBu) in step 6 is replaced with Fmoc-Cya. In addition, peptide cyclization (forming the disulfide bond) is carried out on resin using 10 equivalents of iodine in DMF at room temperature for 2 h.

### EXAMPLE 28

### Synthesis of Compound No. 27: Ac-D-Arg-cyclo[Cys-Cya-His-D-Phe-Arg-Trp-Cys]-NH₂

Can be prepared according to Example 1, with the exception that Fmoc-Glu(OtBu) in step 6 and Fmoc-Arg(pbf) in step 8 are replaced with Fmoc-Cya and Fmoc-D-Arg(pbf), respectively. In addition, peptide cyclization is carried out on resin using 10 equivalents of iodine in DMF at room temperature for 2 h.

### EXAMPLE 29

### Synthesis of Compound No. 28: Ac-Tyr-Arg-cyclo[Cys-Cya-His-D-Phe-Arg-Trp-Cys]-NH₂

Can be prepared according to Example 1, with the exception that Fmoc-Glu(OtBu) in step 6 is replaced with Fmoc-Cya. Fmoc-Tyr(tBu) is added between steps 8 and 9. In addition, peptide cyclization is carried out on resin using 10 equivalents of iodine in DMF for 2 h at room temperature.

### EXAMPLE 30

### Synthesis of Compound No. 29: cyclo[Cys-Glu-His-D-Phe-Arg-Trp-Cys]-NH₂

Can be prepared according to Example 1, with the exception that steps 8 and 9 are omitted.

### EXAMPLE 31

### Synthesis of Compound No. 30: Ac-cyclo[Cys-Glu-His-D-Phe-Arg-Trp-Cys]-NH₂

Can be prepared according to Example 1, with the exception that Fmoc-Arg(Pbf) is not used in step 8.

### EXAMPLE 32

### Synthesis of Compound No. 31: Ac-cyclo[Cys-Glu-His-(4-F-D-Phe)-Arg-Trp-Cys]-NH₂

Can be prepared according to Example 1, with the exception that Fmoc-Arg(Pbf) in step 8 is not used. In addition, Fmoc-4-F-D-Phe is used in step 4 instead of Fmoc-D-Phe.

### EXAMPLE 33

### Synthesis of Compound No. 32: Ac-cyclo[Cys-Glu-His-(4-Cl-D-Phe)-Arg-Trp-Cys]-NH₂

Can be prepared according to Example 1, with the exception that Fmoc-4-Cl-D-Phe is used in step 4 instead of Fmoc-D-Phe. Fmoc-Arg(Pbf) is not used in step 8.

### EXAMPLE 34

### Synthesis of Compound No. 33: Ac-cyclo[Cys-Glu-His-(4-Br-D-Phe)-Arg-Trp-Cys]-NH₂

Can be prepared according to Example 1, with the exception that Fmoc-Arg(Pbf) in step 8 is not used. In addition, Fmoc-4-Br-D-Phe is used instead of Fmoc-D-Phe.

### EXAMPLE 35

### Synthesis of Compound No. 34: Ac-cyclo[Cys-Glu-(1-Me-His)-D-Phe-Arg-Trp-Cys]-NH₂

Can be prepared according to Example 1, with the exception that Fmoc-1-Me-His is used in step 5 instead of Fmoc-His(Trt). Fmoc-Arg(Pbf) in step 8 is omitted.

### EXAMPLE 36

### Synthesis of Compound No. 35: Ac-cyclo[Cys-Glu-His-D-Phe-Arg-Trp-Cys]-Lys-Pro-NH₂

Can be prepared according to Example 1, with the exception that Fmoc-Lys(Boc) and Fmoc-Pro are used prior to step 1. Fmoc-Arg(Pbf) is not used in step 8.

### EXAMPLE 37

### Synthesis of Compound No. 36: Ac-cyclo[Cys-Glu-His-D-Phe-Arg-Trp-Cys]-Ser-Pro-NH₂

Can be prepared according to Example 1, with the exception that Fmoc-Ser and Fmoc-Pro are used prior to step 1. Fmoc-Arg(Pbf) is not used in step 8.

### EXAMPLE 38

### Synthesis of Compound No. 37: N-propionyl-cyclo[Cys-Glu-His-D-Phe-Arg-Trp-Cys]-NH₂

Can be prepared according to Example 1, with the exception that step 8 is not carried out. In addition, step 9 is carried out with propionic acid/DCC/HOBt instead of acetic anhydride.

### EXAMPLE 39

### Synthesis of Compound No. 38: N-butyryl-cyclo[Cys-Glu-His-D-Phe-Arg-Trp-Cys]-NH₂

Can be prepared according to Example 1, with the exception that step 8 is not carried out. In addition, step 9 is carried out with butyric acid/DCC/HOBt instead of acetic anhydride.

### EXAMPLE 40

### Synthesis of Compound No. 39: N-valeryl-cyclo[Cys-Glu-His-D-Phe-Arg-Trp-Cys]-NH₂

Can be prepared according to Example 1, with the exception that step 8 is not carried out. In addition, step 9 is carried out with valerianic acid/DCC/HOBt instead of acetic anhydride.

### EXAMPLE 41

### Synthesis of Compound No. 40: 3-guanidinopropionyl-cyclo[Cys-Glu-His-D-Phe-Arg-Trp-Cys]-NH₂

The peptide resin Cys(Trt)Glu(OtBu)His(Trt)-D-Phe-Arg(Pbf)Trp(Boc)Cys(Trt)-Rink-PS is assembled by standard Fmoc chemistry as previously described. The resin is then treated with a threefold excess of commercially obtained FmocHNCH₂CH₂COOH activated with DCC/HOBt in DMF for 1.5 hrs. The Fmoc group is removed with 30% piperidine in DMF, and the resin washed with additional DMF and DCM. The resin is then suspended in NMP and treated with 2.0 equivalents of N,N-di(Boc)-1-guanylpyrazole and 2.0 equivalents of DIEA in NMP and shaken overnight at room temperature. (Bernatowicz, Wu, and Matsueda, J. Org. Chem. 57(8):2497-2502, 1992).

The resin is washed extensively with NMP, DCM, and MeOH. A subsequent ninhydrin test for free amine is negative. The resin is cleaved, deprotected, and the resulting peptide cyclized and purified as previously described.

### EXAMPLE 42

### Synthesis of Compound No. 41: 4-guanidinobutyryl-cyclo[Cs-Glu-His-D-Phe-Arg-Trp-Cys]-NH₂

The peptide is prepared as in Example 40 above with the exception that FmocHNCH₂CH₂CH₂COOH is utilized in place of Fmoc-HNCH₂CH₂COOH.

### EXAMPLE 43

### Synthesis of Compound No. 42: 5-guanidinovaleryl-cyclo[Cys-Glu-His-D-Phe-Arg-Trp-Cys]-NH₂

The peptide is prepared as in Example 40 above with the exception that FmocHNCH₂CH₂CH₂CH₂COOH is utilized in place of FmocHNCH₂CH₂COOH.

### EXAMPLE 44

### Synthesis of Compound No. 43: Ac-Day-cyclo[Cys-Glu-His-D-Phe-Arg-Trp-Cys]-NH₂

Can be prepared according to Example 1, with the exception that the ArgCysGluHis-D-PheArgTrpCys resin is not treated with acetic anhydride, but instead with 3.0 equivalents of N-α-Fmoc-N-β-tBoc-L-diaminopropionic acid activated with DCC/HOBt. The N-terminal Fmoc group is removed by treatment with 30% piperidine in DMF. The free N-terminus is treated with 5 equivalents of acetic anhydride and 10 equivalents DIEA in dry DMF for 1 hour at room temperature. Resin cleavage, cyclization, and purification are carried out as in Example 1.

### EXAMPLE 45

### Synthesis of Compound No. 44: Ac-Dab-cyclo[Cys-Glu-His-D-Phe-Arg-Trp-Cys]-NH₂

Can be prepared according to Example 1, with the exception that the Arg-Cys-Glu-His-D-Phe-Arg-Trp-Cys resin is not treated with acetic anhydride, but instead with 3.0 equivalents of N-α-Fmoc-N-γ-tBoc-L-diaminobutyric acid activated with DCC/HOBt. The N-terminal Fmoc group is removed by treatment with 30% piperidine in DMF. The free N-terminus is treated with 5 equivalents of acetic anhydride and 10 equivalents DIEA in dry DMF for 1 hour at room temperature. Resin cleavage, cyclization, and purification are carried out as in Example 1.

### EXAMPLE 46

### Synthesis of Compound No. 45: Arg-cyclo[Cys-Glu-His-D-Phe-Arg-Trb-Cys]-OH

Can be prepared according to Example 1, with the exception that acetylation with acetic anhydride in step 9 is not used. In addition, Wang resin is used instead of Rink resin.

### EXAMPLE 47

### Synthesis of Compound No. 46: D-Arg-cyclo[Cys-Glu-His-D-Phe-Arg-Trp-Cys]-NH₂

Can be prepared according to Example 1, with the exception that Fmoc-Arg(pbf) in step 8 is replaced with Fmoc-D-Arg(pbf). In addition, step 9 of acetylation with acetic acid anhydride is not carried out.

### EXAMPLE 48

### Synthesis of Compound No. 47: Ac-D-Arg-cyclo[Cys-Glu-His-Phe-Arg-Trp-Cys]-NH₂

Can be prepared according to Example 1, with the exception that Fmoc-D-Phe in step 4 and Fmoc-Arg(pbf) in step 8 are replaced with Fmoc-Phe and Fmoc-D-Arg(pbf), respectively.

### EXAMPLE 49

### Synthesis of Compound No. 48: Ac-Arg-cyclo[Cys-Glu-His-D-Phe-Arg-Trp-Cys]-NH₂

Can be prepared according to Example 1.

### EXAMPLE 50

### Synthesis of Compound No. 49: Ac-Arg-cyclo[Cys-Glu-His-D-Phe-Arg-Trp-Cys]-OH

Can be prepared according to Example 1, with the exception that Wang resin is used instead of Rink resin.

### EXAMPLE 51

### Synthesis of Compound No. 50: Ac-Arg-cyclo[Cys-Glu-His-(4-Cl-D-Phe)-Arg-Trp-Cys]-NH₂

Can be prepared according to Example 1, with the exception that Fmoc-4-Cl-D-Phe is used in step 4 instead of Fmoc-D-Phe.

### EXAMPLE 52

### Synthesis of Compound No. 51: Ac-Arg-cyclo[Cys-Glu-(1-Me-His)-D-Phe-Arg-Trp-Cys]-NH₂ and Synthesis of Ac-Arg-cyclo[Cys-Glu-(1-Me-D-His)-D-Phe-Arg-Trp-Cys]-NH₂

Can be prepared according to Example 1, with the exception that Fmoc-(1-Me-His) is used in step 5 instead of Fmoc-His(Trt). Due to the unprotected side chain of Fmoc-(1-Me-His), this residue is racemerized during the coupling, which affords two peptides:
Ac-Arg-cyclo[Cys-Glu-(1-Me-His)-D-Phe-Arg-Trp-Cys]-NH₂ and Ac-Arg-cyclo[Cys-Glu-(1-Me-D-His)-D-Phe-Arg-Trp-Cys]-NH₂

The two peptide-isomers are easily separated on HPLC. The absolute configurations of the 1-Me-His residue in each peptide are defined by two-dimensional NMR techniques with proper standard peptides and controls.

### EXAMPLE 53

### Synthesis of Compound No. 52: Ac-D-Arg-cyclo[Cys-Glu-His-D-Phe-Arg-Trp-Cys]-NH₂

Can be prepared according to Example 1, with the exception that Fmoc-D-Arg(Pbf) is used in step 8 instead of Fmoc-Arg(Pbf).

### EXAMPLE 54

### Synthesis of Compound No. 53: Ac-D-Arg-cyclo[Cys-Glu-His-D-Phe-Arg-Trp-Cys]-OH

Can be prepared according to Example 1, with the exception that Fmoc-D-Arg(pbf) is used instead of Fmoc-Arg(pbf) in step 8. In addition, Wang resin is used instead of Rink resin.

### EXAMPLE 55

### Synthesis of Compound No. 54: Ac-hArg-cyclo[Cys-Glu-His-D-Phe-Arg-Trp-Cys]-NH₂

Can be prepared according to Example 1, with the exception that Fmoc-hArg(Pbf) is used in step 8 instead of Fmoc-Arg(Pbf).

### EXAMPLE 56

### Synthesis of Compound No. 55: Ac-Cit-cyclo[Cys-Glu-His-D-Phe-Arg-Trp-Cys]-NH₂

Can be prepared according to Example 1, with the exception that Fmoc-Cit is used in step 8 instead of Fmoc-Arg(Pbf).

### EXAMPLE 57

### Synthesis of Compound No. 56: Ac-Cit-cyclo[Cys-Glu-(1-Me-His)-D-Phe-Arg-Trp-Cys]-NH₂ and Synthesis of Ac-Cit-cyclo[Cys-Glu-(1-Me-D-His)-D-Phe-Arg-Try-Cys]-NH₂

Can be prepared according to Example 1, with the exception that Fmoc-1-Me-His is used in step 5 instead of Fmoc-His(Trt). Fmoc-Cit is used instead of Fmoc-Arg(Pbf) in step 8. Due to the unprotected side chain of Fmoc-(1-Me-His), this residue is racemerized during the coupling, which affords two peptides:
Ac-Cit-cyclo[Cys-Glu-(1-Me-His)-D-Phe-Arg-Trp-Cys]-NH₂ and Ac-Cit-cyclo[Cys-Glu-(1-Me-D-His)-D-Phe-Arg-Trp-Cys]-NH₂.

The two peptide-isomers are easily separated on HPLC. The absolute configurations of the 1-Me-His residue in each peptide are defined by two-dimensional NMR techniques with proper standard peptides and controls.

### EXAMPLE 58

### Synthesis of Compound No. 57: Ac-Leu-cyclo[Cys-Glu-His-D-Phe-Arg-Trp-Cys]-NH₂

Can be prepared according to Example 1, with the exception that Fmoc-Leu is used instead of Fmoc-Arg(Pbf) in step 8.

### EXAMPLE 59

### Synthesis of Compound No. 58: Ac-Lys-cyclo[Cys-Glu-His-D-Phe-Arg-Trp-Cys]-NH₂

Can be prepared according to Example 1, with the exception that Fmoc-Lys(Boc) is used in step 8 instead of Fmoc-Arg(Pbf).

### EXAMPLE 60

### Synthesis of Compound No. 59: Ac-Lys(ipr)-cyclo[Cys-Glu-His-D-Phe-Arg-Trp-Cys]-NH₂

Can be prepared according to Example 1 with the exception that Fmoc-Lys(ipr)(Boc) is used in step 8 instead of Fmoc-Arg(Pbf).

### EXAMPLE 61

### Synthesis of Compound No. 60: Ac-nLeu-cyclo[Cys-Glu-His-D-Phe-Arg-Trp-Cys]-NH₂

Can be prepared according to Example 1, with the exception that Fmoc-nLeu is used instead of Fmoc-Arg(Pbf) in step 8.

### EXAMPLE 62

### Synthesis of Compound No. 61: Ac-nLeu-cyclo[Cys-Glu-His-D-Phe-Arg-Trp-Cys]-Ser-Pro-NH₂

Can be prepared according to Example 1, with the exception that Fmoc-Ser and Fmoc-Pro are used prior to step 1. In addition, Fmoc-nLeu is used instead of Fmoc-Arg(Pbf) in step 8.

### EXAMPLE 63

### Synthesis of Compound No. 62: Ac-Orn-cyclo[Cys-Glu-His-D-Phe-Arg-Trp-Cys]-NH₂

Can be prepared according to Example 1, with the exception that Fmoc-Orn is used in step 8 instead of Fmoc-Arg(Pbf).

### EXAMPLE 64

### Synthesis of Compound No. 63: Ac-Val-cyclo[Cys-Glu-His-D-Phe-Arg-Trp-Cys]-NH₂

Can be prepared according to Example 1, with the exception that Fmoc-Val is used instead of Fmoc-Arg(Pbf) in step 8.

### EXAMPLE 65

### Synthesis of Compound No. 64: N-(2-naphthalenesulfonyl)-D-Arg-cyclo[Cys-Glu-His-D-Phe-Arg-Trp-Cys]-NH₂

Can be prepared according to Example 1, with the exception that Fmoc-Arg(pbf) in step 8 and acetic anhydride in step 9 are replaced with Fmoc-D-Arg(pbf) and 2-naphthalenesulfonylchloride, respectively.

### EXAMPLE 66

### Synthesis of Compound No. 65: N-(4-(2-naphthalenesulfonamido)-4-oxo-butyryl)-D-Arg-cyclo[Cys-Glu-His-D-Phe-Arg-Trp-Cys]-NH₂

Can be prepared according to Example 1, with the exception that Fmoc-Arg(pbf) in step 8 and acetic anhydride in step 9 are replaced with Fmoc-D-Arg(pbf) and succinic anhydride, respectively. Attaching the naphthalene 2′-sulfonamide is carried out as follows: after step 9, the resin is swollen in DCM and washed several times with dry DMF. Then, 5 equivalents of naphthalene 2′-sulfonamide, 10 equivalents of PyBOP, and 10 equivalents of DIEA in dry DMF are added to the resin with a catalytic amount of DMAP (4-(N,N′-dimethylamino)pyridine). The coupling reaction is allowed to proceed at room temperature for 3 h, and the resin is washed and dried.

### EXAMPLE 67

### Synthesis of Compound No. 66: 3-(4-hydroxyphenyl)propionyl-Arg-cyclo[Cys-Glu-His-D-Phe-Arg-Trp-Cys]-NH₂

Can be prepared according to Example 1, with the exception that the Arg-Cys-Glu-His-D-Phe-Arg-Trp-Cys resin is not treated with acetic anhydride, but instead with an excess of 3-(4-hydroxyphenyl)propionic acid activated with DCC/HOBt. The cyclization and purification are carried out as in Example 1.

### EXAMPLE 68

### Synthesis of Compound No. 67: 3-(4-methylbenzoyl)propionyl-Arg-cyclo[Cys-Glu-His-D-Phe-Arg-Trp-Cys]-NH₂

Can be prepared according to Example 1, with the exception that the Arg-Cys-Glu-His-D-Phe-Arg-Trp-Cys resin is not treated with acetic anhydride, but instead with an excess of 3-(4-methylbenzoyl)propionic acid activated with DCC/HOBt. The cyclization and purification are carried out as in Example 1.

### EXAMPLE 69

### Synthesis of Compound No. 68: Tyr-Arg-cyclo[Cys-Glu-His-D-Phe-Arg-Trp-Cys]-NH₂

Can be prepared according to Example 1, with the exception that acetylation with acetic anhydride in step 9 is not used. Fmoc-Tyr(tBu) is added after step 8.

### EXAMPLE 70

### Synthesis of Compound No. 69: Tyr-Arg-cyclo[Cys-Glu-His-D-Phe-Arg-Trp-Cys]-OH

Can be prepared according to Example 1, with the exception that acetylation with acetic anhydride in step 9 is not used. Fmoc-Tyr(tBu) is added after step 8. In addition, Wang resin is used instead of Rink resin.

### EXAMPLE 71

### Synthesis of Compound No. 70: Tyr-Arg-cyclo[Cys-Glu-His-D-Phe-Arg-Trp-Cys]-NH—(CH₂)₆—NH₂

Can be prepared according to Example 1, with the exception that 1,6-diaminohexane trityl resin (Nash, Bycroft, and Chan, Tet. Lett. 37(15):2625-28, 1996) is used instead of Rink resin. In addition, step 9 is not carried out.

### EXAMPLE 72

### Synthesis of Compound No. 71: Tyr-Arg-cyclo[Cys-Glu-His-D-Phe-Arg-Trp-Cys]-Glu-NH₂

Can be prepared according to Example 1, with the exception that Fmoc-Glu is used prior to step 1. Fmoc-Tyr(tBu) is added after step 8. Acetylation with acetic anhydride in step 9 is omitted.

### EXAMPLE 73

### Synthesis of Compound No. 72: Ac-Tyr-Arg-cyclo[Cys-Glu-His-D-Phe-Arg-Trp-Cys]-NH₂

Can be prepared according to. Example 1, with the exception that Fmoc-Tyr(tBu) is added between steps 8 and 9.

### EXAMPLE 74

### Synthesis of Compound No. 73: Ac-Tyr-Arg-cyclo[Cys-Glu-His-D-Phe-Arg-Trp-Cys]-OH

Can be prepared according to Example 1, with the exception that Fmoc-Tyr(tBu) is added between steps 8 and 9. Wang resin is used instead of Rink resin.

### EXAMPLE 75

### Synthesis of Compound No. 74: N-succinyl-Tyr-Arg-cyclo[Cys-Glu-His-D-Phe-Arg-Trp-Cys]-NH₂

Can be prepared according to Example 1, with the exception that step 9 is carried out with succinyl anhydride instead of acetic anhydride. Fmoc-Tyr(tBu) is added between steps 8 and 9.

### EXAMPLE 76

### Synthesis of Compound No. 75: N-glutaryl-Tyr-Arg-cyclo[Cys-Glu-His-D-Phe-Arg-Trp-Cys]-NH₂

Can be prepared according to Example 1, with the exception that step 9 is carried out with glutaryl anhydride instead of acetic anhydride. Fmoc-Tyr(tBu) is added between steps 8 and 9.

### EXAMPLE 77

### Synthesis of Compound No. 76: N-glutaryl-Tyr-Arg-cyclo[Cys-Glu-His-D-Phe-Arg-Trp-Cys]-OH

Can be prepared according to Example 1, with the exception that step 9 is carried out with glutaryl anhydride instead of acetic anhydride. Fmoc-Tyr(tBu) is added between steps 8 and 9. Wang resin is used instead of Rink resin.

### EXAMPLE 78

### Synthesis of Compound No. 77: N-gluconoyl-Tyr-Arg-cyclo[Cys-Glu-His-D-Phe-Arg-Trp-Cys]-NH₂

Can be prepared according to Example 1, with the exception that step 9 is not carried out. Fmoc-Tyr(tBu) is added between steps 8 and 9. The peptide is dissolved in DMF and reacted with gluconolactone/DMAP overnight. The final product is then purified.

### EXAMPLE 79

### Synthesis of Compound No. 78: Ac-Tyr-Arg-cyclo[Cys-Glu-His-D-Phe-Arg-Trp-Cys]-alcohol

Commercially available Fmoc-Cys(Trt)alcohol is attached to commercially available trichloroacetimidate derivatized Wang resin according to published procedure (Yan and Mayer, J. Org. Chem. 68(3):1161-62, 2003). The peptide chain is then extended in the conventional manner to obtain the resin-bound Tyr-Arg-Cys-Glu-His-D-Phe-Arg-Trp-Cys alcohol sequence. Acetylation of the α-amino group is carried out as above with 5 equivalents of acetic anhydride and 10 equivalents DIEA in dry DMF for 1 hour at room temperature. Resin cleavage, cyclization, and purification are carried out as in the above examples.

### EXAMPLE 80

### Synthesis of Compound No. 79: Ac-Tyr-D-Arg-cyclo[Cys-Glu-His-D-Phe-Arg-Trp-Cys]-NH₂

Can be prepared according to Example 1, with the exception that Fmoc-D-Arg(Pbf) is used instead of Fmoc-Arg(Pbf) in step 8. Fmoc-Tyr(tBu) is added between steps 8 and 9.

### EXAMPLE 81

### Synthesis of Compound No. 80: Ac-Tyr-Arg-cyclo[dCys-Glu-His-D-Phe-Arg-Trp-Cys]-NH₂

Can be prepared according to Example 1, with the exception that Fmoc-D-Cys is used in step 7 instead of Fmoc-Cys(Trt). Fmoc-Tyr(tBu) is added between steps 8 and 9.

### EXAMPLE 82

### Synthesis of Compound No. 81: Ac-Tyr-Arg-cyclo[Cys-Glu-(1-Me-His)-D-Phe-Arg-Trp-Cys]-NH₂ and Synthesis of Compound No. 82: Ac-Tyr-Arg-cyclo[Cys-Glu-(1-Me-D-His)-D-Phe-Arg-Trp-Cys]-NH₂

Can be prepared according to Example 1, with the exception that Fmoc-(1-Me-His) is used in step 5 instead of Fmoc-His(Trt). In addition, Fmoc-Tyr(tBu) is added between steps 8 and 9. Due to the unprotected side chain of Fmoc-(1-Me-His), this residue is racemerized during the coupling, which affords two peptides:
Ac-Tyr-Arg-cyclo[Cys-Glu-(1-Me-His)-D-Phe-Arg-Trp-Cys]-NH₂ and Ac-Tyr-Arg-cyclo[Cys-Glu-(1-Me-D-His)-D-Phe-Arg-Trp-Cys]-NH₂.

The two peptide-isomers are easily separated on HPLC. The absolute configurations of the 1-Me-His residue in each peptide are defined by two-dimensional NMR techniques with proper standard peptides and controls.

### EXAMPLE 83

### Synthesis of Compound No. 84: Ac-Tyr-Arg-cyclo[Cys-Glu-(1-Me-His)-(4-F-D-Phe)-Arg-Trp-Cys]-NH₂ and Synthesis of Compound No. 85: Ac-Tyr-Arg-cyclo[Cys-Glu-(1-Me-D-His)-(4-F-D-Phe)-Arg-Trp-Cys]-NH₂

Can be prepared according to Example 1, with the exception that Fmoc-1-Me-His is used in step 5 instead of Fmoc-His(Trt). Fmoc-4-F-D-Phe is used instead of Fmoc-D-Phe in step 4. Fmoc-Tyr(tBu) is added between steps 8 and 9. Due to the unprotected side chain of Fmoc-(1-Me-His), this residue is racemerized during the coupling, which affords two peptides:
Ac-Tyr-Arg-cyclo[Cys-Glu-(1-Me-His)-(4-F-D-Phe)-Arg-Trp-Cys]-NH₂ and Ac-Tyr-Arg-cyclo[Cys-Glu-(1-Me-D-His)-(4-F-D-Phe)-Arg-Trp-Cys]-NH₂.

The two peptide-isomers are easily separated on HPLC. The absolute configurations of the 1-Me-His residue in each peptide are defined by two-dimensional NMR techniques with proper standard peptides and controls.

### EXAMPLE 84

### Synthesis of Compound No. 86: Ac-Tyr-Arg-cyclo[Cys-Glu-His-(4-Cl-D-Phe)-Arg-Trp-Cys]-NH₂

Can be prepared according to Example 1, with the exception that Fmoc-4-Cl-D-Phe is used in step 4 instead of Fmoc-D-Phe. In addition, Fmoc-Tyr(tBu) is added between steps 8 and 9.

### EXAMPLE 85

### Synthesis of Compound No. 87: Ac-Tyr-Arg-cyclo[Cys-Glu-(1-Me-His)-(4-Cl-D-Phe)-Arg-Trp-Cys]-NH₂ and Synthesis of Compound No. 88: Ac-Tyr-Arg-cyclo[Cys-Glu-(1-Me-D-His)-(4-Cl-D-Phe)-Arg-Trp-Cys]-NH₂

Can be prepared according to Example 1, with the exception that Fmoc-4-Cl-D-Phe is used in step 4 instead of Fmoc-D-Phe and Fmoc-(1-Me-His) is used in step 5 instead of Fmoc-His(Trt), respectively. In addition, Fmoc-Tyr(tBu) is added between steps 8 and 9. Due to the unprotected side chain of Fmoc-(1-Me-His), this residue is racemerized during the coupling, which affords two peptides:
Ac-Tyr-Arg-cyclo[Cys-Glu-(1-Me-His)-(4-Cl-D-Phe)-Arg-Trp-Cys]-NH₂ and Ac-Tyr-Arg-cyclo[Cys-Glu-(1-Me-DHis)-(4-Cl-D-Phe)-Arg-Trp-Cys]-NH₂.

The two peptide-isomers are easily separated on HPLC. The absolute configurations of the 1-Me-His residue in each peptide are defined by two-dimensional NMR techniques with proper standard peptides and controls.

### EXAMPLE 86

### Synthesis of Compound No. 89: Ac-Tyr-Arg-cyclo[Cys-Glu-His-(4-Br-D-Phe)-Arg-Trp-Cys]-NH₂

Can be prepared according to Example 1, with the exception that Fmoc-4-Br-D-Phe is used instead of Fmoc-D-Phe in step 4. Fmoc-Tyr(tBu) is added between steps 8 and 9.

### EXAMPLE 87

### Synthesis of Compound No. 90: Ac-Tyr-Arg-cyclo[Cys-Glu-(1-Me-His)-(4-Br-D-Phe)-Arg-Trp-Cys]-NH₂ and Synthesis of Compound No. 91: Ac-Tyr-Arg-cyclo[Cys-Glu-(1-Me-D-His)-(4-Br-D-Phe)-Arg-Trp-Cys]-NH₂

Can be prepared according to Example 1, with the exception that Fmoc-4-Br-D-Phe is used in step 4 instead of Fmoc-D-Phe and Fmoc-(1-Me-His) is used in step 5 instead of Fmoc-His(Trt), respectively. In addition, Fmoc-Tyr(tBu) is added between steps 8 and 9. Due to the unprotected side chain of Fmoc-(1-Me-His), this residue is racemerized during the coupling, which affords two peptides:
Ac-Tyr-Arg-cyclo[Cys-Glu-(1-Me-His)-(4-Br-D-Phe)-Arg-Trp-Cys]-NH₂ and Ac-Tyr-Arg-cyclo[Cys-Glu-(1-Me-D-His)-(4-Br-D-Phe)-Arg-Trp-Cys]-NH₂.

The two peptide-isomers are easily separated on HPLC. The absolute configurations of the 1-Me-His residue in each peptide are defined by two-dimensional NMR techniques with proper standard peptides and controls.

### EXAMPLE 88

### Synthesis of Compound No. 92: Ac-Tyr-Arg-cyclo[Cys-Glu-His-(4-Me-D-Phe)-Arg-Trp-Cys]-NH₂

Can be prepared according to Example 1, with the exception that Fmoc-4-Me-D-Phe is used in step 4 instead of Fmoc-D-Phe. Fmoc-Tyr(tBu) is added between en steps 8 and 9.

### EXAMPLE 89

### Synthesis of Compound No. 93: Ac-Tyr-Arg-cyclo[Cys-Glu-His-(4-OMe-D-Phe)-Arg-Trp-Cys]-NH₂

Can be prepared according to Example 1, with the exception that Fmoc-4-OMe-D-Phe is used in step 4 instead of Fmoc-D-Phe. Fmoc-Tyr(tBu) is added between steps 8 and 9.

### EXAMPLE 90

### Synthesis of Compound No. 94: Ac-Tyr-Arg-cyclo[Cys-Glu-(1-Me-His)-(4-OMe-D-Phe)-Arg-Trp-Cys]-NR₂ and Synthesis of Compound No. 95: Ac-Tyr-Arg-cyclo[Cys-Glu-(1-Me-D-His)-(4-OMe-D-Phe)-Arg-Trp-Cys]-NH₂

Can be prepared according to Example 1, with the exception that Fmoc-1-Me-His is used in step 5 instead of Fmoc-His(Trt). Fmoc-4-OMe-D-Phe is used instead of Fmoc-D-Phe in step 4. Fmoc-Tyr(tBu) is added between steps 8 and 9. Due to the unprotected side chain of Fmoc-(1-Me-His), this residue i s racemerized during the coupling, which affords two peptides:
Ac-Tyr-Arg-cyclo[Cys-Glu-(1-Me-His)-(4-OMe-D-Phe)-Arg-Trp-Cys]-NR₂ and Ac-Tyr-Arg-cyclo[Cys-Glu-(1-Me-D-His)-(4-OMe-D-Phe)-Arg-Trp-Cys]-NR₂.

The two peptide-isomers are easily separated on HPLC. The absolute configurations of the 1-Me-His residue in each peptide are defined by two-dimensional NMR techniques with proper standard peptides and controls.

### EXAMPLE 91

### Synthesis of Compound No. 96: Ac-Tyr-Arg-cyclo[Cys-Glu-(3-Me-His)-D-Phe-Arg-Trp-Cys]-NH₂

Can be prepared according to Example 1, with the exception that Fmoc-3-Me-His is used in step 5 instead of Fmoc-His(Trt). Fmoc-Tyr(tBu) is added between steps 8 and 9.

### EXAMPLE 92

### Synthesis of Compound No. 99: Ac-Tyr-Arg-cyclo[Cys-Glu-(1-Bzl-His)-D-Phe-Arg-Trp-Cys]-NH₂ Synthesis of Compound No. 100: Ac-Tyr-Arg-cyclo[Cys-Glu-(1-Bzl-D-His)-DPhe-Arg-Trp-Cys]-NH₂

Can be prepared according to Example 1, with the exception that Fmoc-1-Bzl-His is used in step 5 instead of Fmoc-His(Trt). Fmoc-Tyr(tBu) is added between steps 8 and 9. Due to the unprotected side chain of Fmoc-(1-Bzl-His), this residue is racemerized during the coupling, which affords two peptides:
Ac-Tyr-Arg-cyclo[Cys-Glu-(1-Bzl-His)-D-Phe-Arg-Trp-Cys]-NH₂ and Ac-Tyr-Arg-cyclo[Cys-Glu-(1-Bzl-D-His)-D-Phe-Arg-Trp-Cys]-NH₂

The two peptide-isomers are easily separated on HPLC. The absolute configurations of the 1-Bzl-His residue in each peptide are defined by two-dimensional NMR techniques with proper standard peptides and controls.

### EXAMPLE 93

### Synthesis of Compound No. 101: Ac-Tyr-Arg-cyclo[Cys-Glu-(1-Bom-His)-D-Phe-Arg-Trp-Cys]-NH₂

Can be prepared according to Example 1, with the exception that Fmoc-1-Bom-His is used in step 5 instead of Fmoc-His(Trt). Fmoc-Tyr(tBu) is added between steps 8 and 9.

### EXAMPLE 94

### Synthesis of Compound No. 110: Ac-Tyr-Arg-cyclo[Cys-Glu-(β-(2-furyl)-Ala)-D-Phe-Arg-Trp-Cys]-NH₂

Can be prepared according to Example 1, with the exception that Fmoc-β-(2-furyl)-Ala is used in step 5 instead of Fmoc-His(Trt). In addition, Fmoc-Tyr(tBu) is added between steps 8 and 9.

### EXAMPLE 95

### Synthesis of Compound No. 111: Ac-Tyr-Arg-cyclo[Cys-Glu-(β-(thien-2-yl)-Ala)-D-Phe-Arg-Trp-Cys]-NH₂

Can be prepared according to Example 1, with the exception that Fmoc-β-(thien-2-yl)-Ala is used in step 5 instead of Fmoc-His(Trt). In addition, Fmoc-Tyr(tBu) is added between steps 8 and 9.

### EXAMPLE 96

### Synthesis of Compound No. 112: Ac-Tyr-Arg-cyclo[Cys-Glu-(β-(1,3-thiazol-4-yl)-Ala)-D-Phe-Arg-Trp-Cys]-NH₂

Can be prepared according to, Example 1, with the exception that Fmoc-β-(1,3-thiazol-4-yl)-Ala is used in step 5 instead of Fmoc-His(Trt). In addition, Fmoc-Tyr(tBu) is added between steps 8 and 9.

### EXAMPLE 97

### Synthesis of Compound No. 113: Ac-Tyr-Arg-cyclo[Cys-Glu-(β-(pyridin-4-yl)-Ala)-D-Phe-Arg-Try-Cys]-NH₂

Can be prepared according to Example 1, with the exception that Fmoc-β-(pyridin-4-yl)-Ala is used in step 5 instead of Fmoc-His(Trt). In addition, Fmoc-Tyr(tBu) is added between steps 8 and 9.

### EXAMPLE 98

### Synthesis of Compound No. 114: Ac-Tyr-Arg-cyclo[Cys-Glu-His-D-Phe-Arg-Trp-Cys]-glycinol

Can be prepared according to Example 1, with the exception that glycinol 2-chlorotrityl resin (Barbos, Chatzi, Gatos, and Stavropoulos, Int. J. Pept. Protein Res. 37(6):513-20, 1991) is used instead of Rink resin.

### EXAMPLE 99

### Synthesis of Compound No. 115: Ac-Tyr-Arg-cyclo[Cys-Glu-His-D-Phe-Arg-Trp-Cys]-2-(2-aminoethoxy)ethanol

Can be prepared according to Example 1, with the exception that 2-(2-aminoethoxy) ethanol 2-chlorotrityl resin (Barbos, Chatzi, Gatos, and Stavropoulos, Int. J. Pept. Protein Res. 37(6):513-20, 1991) is used instead of Rink resin.

### EXAMPLE 100

### Synthesis of Compound No. 116: Ac-Tyr-Arg-cyclo[Cys-Glu-His-D-Phe-Arg-Trp-Cys]-Ser alcohol

Can be prepared according to Example 1, with the exception that Wang resin is used instead of Rink resin. Wang resin was preloaded with Fmoc-serinol(tBu) according to a published method (Yan and Mayer, J. Org. Chem. 68:1161-62, 2003) prior to step 1. Tyr(tBu) is used between steps 8 and 9.

### EXAMPLE 101

### Synthesis of Compound No. 117: Ac-Tyr-Arg-cyclo[Cys-Glu-His-D-Phe-Arg-Trp-Cys]-NH—(CH₂)₆—NH₂

Can be prepared according to Example 1, with the exception that 1,6-diaminohexane trityl resin (Nash, Bycroft, and Chan, Tet. Lett. 37(15):2625-28, 1996) is used instead of Rink resin.

### EXAMPLE 102

### Synthesis of Compound No. 118: Ac-Tyr-Arg-cyclo[Cys-Glu-His-D-Phe-Arg-Trp-Cys]-Glu-NH₂

Can be prepared according to Example 1, with the exception that Fmoc-Glu(OtBu) is used prior to step 1. Fmoc-Tyr(tBu) is added between steps 8 and 9.

### EXAMPLE 103

### Synthesis of Compound No. 119: Ac-Tyr-Arg-cyclo[Cys-Glu-His-D-Phe-Arg-Trp-Cys]-Ser-Pro-NH₂

Can be prepared according to Example 1, with the exception that Fmoc-Ser and Fmoc-Pro are used prior to step 1. In addition, Fmoc-Tyr is used between steps 8 and 9.

### EXAMPLE 104

### Synthesis of Compound No. 120: Ac-Tyr-Arg-cyclo[Cys-Glu-His-D-Phe-Arg-Trp-Cys]-Ser-Pro alcohol

Can be prepared according to Example 1, with the exception that Wang resin is used instead of Rink resin. Wang resin was preloaded with Fmoc-prolinol according to a published method (Yan and Mayer, J. Org. Chem. 68:1161-62, 2003), and then Fmoc-Ser(tBu) was added prior to step 1. In addition, Fmoc-Tyr(tBu) is used between steps 8 and 9.

### EXAMPLE 105

### Synthesis of Compound No. 121: Ac-Tyr-Arg-cyclo[Cys-Glu-His-D-Phe-Arg-Trp-Cys]-Lys-Pro-NH₂

Can be prepared according to Example 1, with the exception that Fmoc-Lys(Boc) and Fmoc-Pro are used prior to step 1. Fmoc-Tyr(tBu) is used between steps 8 and 9.

### EXAMPLE 106

### Synthesis of Compound No. 122: Ac-Tyr-Arg-cyclo[Cys-Glu-His-D-Phe-Arg-Trp-Cys]-Lys-Pro alcohol

Can be prepared according to Example 1, with the exception that Wang resin is used instead of Rink resin. Wang resin was preloaded with Fmoc-prolinol according to a published method (Yan and Mayer, J. Org. Chem. 68:1161-62, 2003), and then Fmoc-Lys(Boc) was added prior to step 1. In addition, Fmoc-Tyr(tBu) is used between steps 8 and 9.

### EXAMPLE 107

### Synthesis of Compound No. 123: Ac-Tyr-Arg-cyclo[Cys-Glu-His-D-Phe-Arg-Trp-Cys]-Arg-Phe-NH₂

Can be prepared according to Example 1, with the exception that Fmoc-Arg(Pbf) and Fmoc-Phe are used prior to step 1. Fmoc-Tyr(tBu) is used between steps 8 and 9.

### EXAMPLE 108

### Synthesis of Compound No. 124: Ac-Tyr-Cit-cyclo[Cys-Glu-His-D-Phe-Arg-Trp-Cys]-NH₂

Can be prepared according to Example 1, with the exception that Fmoc-Cit is used instead of Fmoc-Arg(Pbf) in step 8, and Fmoc-Tyr(tBu) is added between steps 8 and 9.

### EXAMPLE 109

### Synthesis of Compound No. 125: Ac-Tyr-Cit-cyclo[Cys-Glu-(1-Me-His)-D-Phe-Arg-Trp-Cys]-NH₂ and Synthesis of Ac-Tyr-Cit-cyclo[Cys-Glu-(1-Me-D-His)-D-Phe-Arg-Trp-Cys]-NH₂

Can be prepared according to Example 1, with the exception that Fmoc-1-Me-His is used in step 5 instead of Fmoc-His(Trt). Fmoc-Cit is used instead of Fmoc-Arg(Pbf) in step 8. Fmoc-Tyr(tBu) is added between steps 8 and 9. Due to the unprotected side chain of Fmoc-(1-Me-His), this residue is racemerized during the coupling, which affords two peptides:
Ac-Tyr-Cit-cyclo[Cys-Glu-(1-Me-His)-D-Phe-Arg-Trp-Cys]-NH₂ and Ac-Tyr-Cit-cyclo[Cys-Glu-(1-Me-D-His)-D-Phe-Arg-Trp-Cys]-NH₂.

The two peptide-isomers are easily separated on HPLC. The absolute configurations of the 1-Me-His residue in each peptide are defined by two-dimensional NMR techniques with proper standard peptides and controls.

### EXAMPLE 110

### Synthesis of Compound No. 126: Ac-Tyr-hArg-cyclo[Cys-Glu-His-D-Phe-Arg-Trp-Cys]-NH₂

Can be prepared according to Example 1, with the exception that Fmoc-hArg(Pbf) is used in step 8 instead of Fmoc-Arg(Pbf). Fmoc-Tyr (OtBu) is added between steps 8 and 9.

### EXAMPLE 111

### Synthesis of Compound No. 127: Ac-Tyr-(1-β-hArg)-cyclo[Cys-Glu-His-D-Phe-Arg-Trp-Cys]-NH₂

Can be prepared according to Example 1, with the exception that Fmoc-1-β-hArg(Pbf) is used instead of Fmoc-Arg(Pbf) in step 8. Fmoc-Tyr(tBu) is used between steps 8 and 9.

### EXAMPLE 112

### Synthesis of Compound No. 128: Ac-Tyr-Lys-cyclo[Cys-Glu-His-D-Phe-Arg-Trp-Cys]-NH₂

Can be prepared according to Example 1, with the exception that Fmoc-Lys(Boc) is used in step 8 instead of Fmoc-Arg(Pbf). Fmoc-Tyr(tBu) is used between steps 8 and 9.

### EXAMPLE 113

### Synthesis of Compound No. 129: Ac-Tyr-Ser-cyclo[Cys-Glu-His-D-Phe-Arg-Trp-Cys]-NH₂

Can be prepared according to Example 1, with the exception that Fmoc-Ser is used instead of Fmoc-Arg(Pbf) in step 8. Fmoc-Tyr(tBu) is used between steps 8 and 9.

### EXAMPLE 114

### Synthesis of Compound No. 130: Ac-Tyr-Val-cyclo[Cys-Glu-His-D-Phe-Arg-Trp-Cys]-NH₂

Can be prepared according to Example 1, with the exception that Fmoc-Val is used instead of Fmoc-Arg(Pbf) in step 8. Fmoc-Tyr(tBu) is used between steps 8 and 9.

### EXAMPLE 115

### Synthesis of Compound No. 131: N-succinyl-Tyr-Arg-cyclo[Cys-Glu-His-D-Phe-Arg-Trp-Cys]-OH

Can be prepared according to Example 1, with the exception that step 9 is carried out with succinyl anhydride instead of acetic anhydride. Fmoc-Tyr(tBu) is added between steps 8 and 9. Wang resin is used instead of Rink resin.

### EXAMPLE 116

### Synthesis of Compound No. 132: cyclo[hCys-His-D-Phe-Arg-Trp-Cys]-NH₂

Can be prepared according to Example 1, with the exception that Fmoc-Glu(OtBu) and Fmoc-Arg(Pbf) in steps 6and 8, respectively, are not used. In addition, acetylation with acetic anhydride in step 9 is not used. Finally, homocysteine is used instead of cysteine in step 7.

### EXAMPLE 117

### Synthesis of Compound No. 133: cyclo[hCys-His-D-Phe-Arg-Trp-Cys]-OH

Can be prepared according to Example 1, with the exception that Fmoc-Glu(OtBu) in step 6, Fmoc-Arg(pbf) in step 8, and acetylation with acetic anhydride in step 9 are not used. Homocysteine is used instead of cysteine in step 7. Wang resin is used instead of Rink resin.

### EXAMPLE 118

### Synthesis of Compound No. 134: cyclo[hCys-His-(4-F-D-Phe)-Arg-Trp-Cys]-NH₂

Can be prepared according to Example 1, with the exception that Fmoc-Glu(OtBu) in step 6, Fmoc-Arg(pbf) in step 8, and acetylation with acetic anhydride in step 9 are not used. In addition, Fmoc-hCys(Trt) is used instead of Fmoc-Cys(Trt) in step 7, and Fmoc-(4-F-D-Phe) is used instead of Fmoc-D-Phe in step 4.

### EXAMPLE 119

### Synthesis of Compound No. 135: cyclo[hCys-His-(4-Cl-D-Phe)-Arg-Trp-Cys]-NH₂

Can be prepared according to Example 1, with the exception that Fmoc-Glu(OtBu) in step 6, Fmoc-Arg(pbf) in step 8, and acetylation with acetic anhydride in step 9 are not used. In addition, Fmoc-hCys(Trt) is used in step 7, and Fmoc-4-Cl-D-Phe is used instead of Fmoc-D-Phe in step 4.

### EXAMPLE 120

### Synthesis of Compound No. 136: Ac-cyclo[hCys-His-Phe-Arg-Trp-Cys]-NH₂

Can be prepared according to Example 1, with the exception that Fmoc-Glu(OtBu) in step 6 and Fmoc-Arg(pbf) in step 8 are not used. In addition, Fmoc-D-Phe in step 4 and Fmoc-Cys(Trt) in step 7 are replaced with Fmoc-Phe and Fmoc-hCys(Trt), respectively.

### EXAMPLE 121

### Synthesis of Compound No. 137: Ac-cyclo[hCys-His-D-Phe-Arg-Trp-Cys]-NH₂

Can be prepared according to Example 1, with the exception that Fmoc-Glu(OtBu) and Fmoc-Arg(Pbf) in steps 6 and 8, respectively, are not used. In addition, homocysteine is used instead of cysteine in step 7.

### EXAMPLE 122

### Synthesis of Compound No. 138: Ac-cyclo[hCys-His-D-Phe-Arg-Trp-Cys]-OH

Can be prepared according to Example 1, with the exception that homocysteine is used instead of cysteine in step 7, and Fmoc-Arg(Pbf) is omitted from step 8. Wang resin is used instead of Rink resin.

### EXAMPLE 123

### Synthesis of Compound No. 139: Ac-cyclo[hCys-His-(4-F-D-Phe)-Arg-Trp-Cys]-NH₂

Can be prepared according to Example 1, with the exception that Fmoc-Glu(OtBu) in step 6 and Fmoc-Arg(pbf) in step 8 are not used. In addition, Fmoc-hCys(Trt) and Fmoc-(4-F-D-Phe) are used instead of Fmoc-Cys(Trt) in step 7 and Fmoc-D-Phe in step 4, respectively.

### EXAMPLE 124

### Synthesis of Compound No. 140: Ac-cyclo[hCys-His-(4-Cl-D-Phe)-Arg-Trp-Cys]-NH₂

Can be prepared according to Example 1, with the exception that Fmoc-Glu(OtBu) and Fmoc-Arg(pbf) in steps 6 and 8 are not used. In addition, Fmoc-hCys(Trt) and Fmoc-4-Cl-D-Phe are used instead of Fmoc-Cys(Trt) and Fmoc-D-Phe, respectively, in steps 4 and 7.

### EXAMPLE 125

### Synthesis of Compound No. 141: N-cyclopropanecarbonyl-cyclo[hCys-His-D-Phe-Arg-Trp-Cys]-NH₂

Can be prepared according to Example 1, with the exception that Fmoc-Glu(OtBu) in step 6 and Fmoc-Arg(pbf) in step 8 are not used. Fmoc-Cys(Trt) in step 7 is replaced with Fmoc-hCys(Trt). In addition, in step 9, acetic acid anhydride is replaced with cyclopropane carboxylic acid, which is pre-activated with DIC (1,3-diisopropyl-carbodiimide)/HOBt (1-hydroxylbenzotriazole).

### EXAMPLE 126

### Synthesis of Compound No. 142: N-cyclobutanecarbonyl-cyclo[hCys-His-D-Phe-Arg-Trp-Cys]-NH₂

Can be prepared according to Example 1, with the exception that Fmoc-Glu(OtBu) in step 6 and Fmoc-Arg(pbf) in step 8 are not used. Fmoc-hCys(Trt) is used in step 7 instead of Fmoc-Cys(Trt). In addition, in step 9, acetic anhydride is replaced with cyclobutane carboxylic acid, which is pre-activated with DIC (1,3-diisopropyl-carbodiimide)/HOBt (1-hydroxylbenzotriazole).

### EXAMPLE 127

### Synthesis of Compound No. 143: N-cyclopentanecarbonyl-cyclo[hCys-His-D-Phe-Arg-Trp-Cys]-NH₂

Can be prepared according to Example 1, with the exception that Fmoc-Glu(OtBu) in step 6 and Fmoc-Arg(pbf) in step 8 are not used. Fmoc-Cys(Trt) in step 7 is replaced with Fmoc-hCys(Trt). In addition, in step 9, acetic acid anhydride is replaced with cyclopentane carboxylic acid, which is pre-activated with DIC (1,3-diisopropyl-carbodiimide)/HOBt (1-hydroxylbenzotriazole).

### EXAMPLE 128

### Synthesis of Compound No. 144: N-cyclohexanecarbonyl-cyclo[hCys-His-D-Phe-Arg-Trp-Cys]-NH₂

Can be prepared according to Example 1, with the exception that Fmoc-Glu(OtBu) in step 6 and Fmoc-Arg(pbf) in step 8 are not used. Fmoc-hCys(Trt) is used in step 7 instead of Fmoc-Cys(Trt). In addition, in step 9, acetic anhydride is replaced with cyclohexane carboxylic acid, which is pre-activated with DIC (1,3-diisopropyl-carbodiimide)/HOBt (1-hydroxylbenzotriazole).

### EXAMPLE 129

### Synthesis of Compound No. 145: N-hexanoyl-cyclo[hCys-His-D-Phe-Arg-Trp-Cys]NH₂

Can be prepared according to Example 1, with the exception that Fmoc-Glu(OtBu) in step 6 and Fmoc-Arg(pbf) in step 8 are not used. Fmoc-hCys(Trt) is used in step 7 instead of Fmoc-Cys(Trt). In addition, in step 9 acetic anhydride is replaced with n-hexanoic acid, which is pre-activated with DIC (1,3-diisopropylcarbodiimide)/HOBt (1-hydroxylbenzotriazole).

### EXAMPLE 130

### Synthesis of Compound No. 146: N-benzoyl-cyclo[hCys-His-D-Phe-Arg-Trp-Cys]-NH₂

Can be prepared according to Example 1, with the exception that Fmoc-Glu(OtBu) in step 6 and Fmoc-Arg(pbf) in step 8 are not used. Fmoc-hCys(Trt) is used in step 7 instead of Fmoc-Cys(Trt). In addition, in step 9, acetic anhydride is replaced with benzoic acid, which is pre-activated with DIC (1,3-diisopropylcarbodiimide)/HOBt (1-hydroxylbenzotriazole).

### EXAMPLE 131

### Synthesis of Compound No. 147: 4-phenylbutyryl-cyclo[hCys-His-D-Phe-Arg-Trp-Cys]-NH₂

Can be prepared according to Example 1, with the exception that Fmoc-Glu(OtBu) in step 6 and Fmoc-Arg(pbf) in step 8 are not used. Fmoc-hCys(Trt) is used in step 7 instead of Fmoc-Cys(Trt). In addition, in step 9, acetic anhydride is replaced with 4-phenylbutyric acid, which is pre-activated with DIC (1,3-diisopropylcarbodiimide)/HOBt (1-hydroxylbenzotriazole).

### EXAMPLE 132

### Synthesis of Compound No. 148: 3-guanidinopropionyl-cyclo[hCys-His-D-Phe-Arg-Trp-Cys]-NH₂

Can be prepared according to Example 1, with the exception that Fmoc-Glu(OtBu) in step 6 is not used. Fmoc-Cys(Trt) in step 7 and Fmoc-Arg(pbf) in step 8 are replaced with Fmoc-hCys(Trt) and Fmoc-β-Ala(Fmoc-3-amino propionic acid), respectively. In addition, step 9, acetylation is replaced the following treatment (guanidylation): After Fmoc deprotection, the resin is incubated with 10 equivalents of N,N′-bis(tert-butoxycarbonyl)-1H-pyrazole-1-carboxamidine and 10 equivalents of DIEA in NMP (N-methylpyrrolidone) overnight at room temperature.

### EXAMPLE 133

### Synthesis of Compound No. 149: 5-guanidinovaleryl-cyclo[hCys-His-D-Phe-Arg-Trp-Cys]-NH₂

Can be prepared according to Example 1, with the exception that Fmoc-Glu(OtBu) in step 6 is not used. Fmoc-Cys(Trt) in step 7 and Fmoc-Arg(pbf) in step 8 are replaced with Fmoc-hCys(Trt) and Fmoc-5-amino-valeric acid, respectively. In addition, step 9, acetylation is replaced the following treatment (guanidylation): After Fmoc deprotection, the resin is incubated with 10 equivalents of N,N′-bis(tert-butoxycarbonyl)-1H-pyrazole-1-carboxamidine and 10 equivalents of DIEA in NMP (N-methylpyrrolidone) overnight at room temperature.

### EXAMPLE 134

### Synthesis of Compound No. 150: N-phenylsulfonyl-cyclo[hCys-His-D-Phe-Arg-Trp-Cys]-NH₂

Can be prepared according to Example 1, with the exception that Fmoc-Glu(OtBu) in step 6 and Fmoc-Arg(pbf) in step 8 are not used. In addition, Fmoc-hCys(Trt) in step 7 used instead of Fmoc-Cys(Trt). Acetic anhydride in step 9 is replaced with phenylsulfonylchloride.

### EXAMPLE 135

### Synthesis of Compound No. 151: N-(2-naphthalenesulfonyl)-cyclo[hCys-His-D-Phe-Arg-Trp-Cys]-NH₂

Can be prepared according to Example 1, with the exception that Fmoc-Glu(OtBu) in step 6 and Fmoc-Arg(pbf) in step 8 are not used. In addition, Fmoc-hCys(Trt) in step 7 used instead of Fmoc-Cys(Trt). Acetic anhydride in step 9 is replaced with 2-naphthalenesulfonylchloride.

### EXAMPLE 136

### Synthesis of Compound No. 152: N-(4-phenylsulfonamido-4-oxo-butyryl)-cyclo[hCys-His-D-Phe-Arg-Try-Cys]-NH₂

Can be prepared according to Example 1, with the exception that Fmoc-Glu(OtBu) in step 6 and Fmoc-Arg(pbf) in step 8 are not used. Fmoc-hCys(Trt) is used in step 7 instead of Fmoc-Cys(Trt). In step 9, acetic anhydride is replaced with succinic acid anhydride. In addition, one more step is added after step 9. Attaching the phenylsulfonamide is as follows: after the step 9, the resin is swollen in DCM and washed several times with dry DMF. Then, 5 equivalents of phenylsulfonamide, 10 equivalents of PyBOP, and 10 equivalents of DIEA in dry DMF are added to the resin with a catalytic amount of DMAP (4-(N,N′-dimethylamino)pyridine). The coupling reaction is allowed to proceed at room temperature for 3 h, and the resin is then washed and dried.

### EXAMPLE 137

### Synthesis of Compound No. 153: Arg-cyclo[hCys-His-D-Phe-Arg-Trp-Cys]-NH₂

Can be prepared according to Example 1, with the exception that Fmoc-Glu(OtBu) and acetylation with acetic anhydride in steps 6 and 9, respectively, are not used. In addition, homocysteine is used instead of cysteine in step 7.

### EXAMPLE 138

### Synthesis of Compound No. 154: D-Arg-cyclo[hCys-His-D-Phe-Arg-Trp-Cys]-NH₂

Can be prepared according to Example 1, with the exception that Fmoc-Arg(pbf) in step 8 is replaced with Fmoc-D-Arg(pbf) and Fmoc-Glu(OtBu), and acetylation with acetic anhydride in steps 6 and 9, respectively, are not used. Finally, homocysteine is used instead of cysteine in step 7.

### EXAMPLE 139

### Synthesis of Compound No. 155: Arg-cyclo[hCys-His-D-Phe-Arg-Trp-Cys]-OH

Can be prepared according to Example 1, with the exception that Fmoc-Glu(OtBu) acetylation with acetic anhydride in steps 6 and 9, respectively, are not used. In addition, Wang resin is used instead of Rink resin. Finally, homocysteine is used instead of cysteine in step 7.

### EXAMPLE 140

### Synthesis of Compound No. 156: Arg-cyclo[hCys-(1-Me-His)-D-Phe-Arg-Trp-Cys]-NH₂ and Synthesis of Compound No. 157: Arg-cyclo[hCys-(1-Me-D-His)-D-Phe-Arg-Trp-Cys]-NH₂

Can be prepared according to Example 1, with the exception that Fmoc-(1-Me-His) is used in step 5 instead of Fmoc-His(Tit). Fmoc-hCys(Trt) is used instead of Fmoc-Cys(Trt) in step 6. In addition, acetylation with acetic anhydride in step 9 is not used. Due to the unprotected side chain of Fmoc-(1-Me-His), this residue is racemerized during the coupling, which affords two peptides:
Arg-cyclo[hCys-(1-Me-His)-D-Phe-Arg-Trp-Cys]-NH₂ and Arg-cyclo[hCys-(1-Me-D-His)-D-Phe-Arg-Trp-Cys]-NH₂.

The two peptide-isomers are easily separated on HPLC. The absolute configurations of the 1-Me-His residue in each peptide are defined by two-dimensional NMR techniques with proper standard peptides and controls.

### EXAMPLE 141

### Synthesis of Compound No. 158: Ac-Arg-cyclo[hCys-His-D-Phe-Arg-Trp-Cys]-NH₂

Can be prepared according to Example 1, with the exception that Fmoc-Glu(OtBu) in step 6 is not used. In addition, homocysteine is used instead of cysteine in step 7.

### EXAMPLE 142

### Synthesis of Compound No. 159: Ac-Arg-cyclo[hCys-His-D-Phe-Arg-Trp-Cys]-OH

Can be prepared according to Example 1, with the exception that Fmoc-Glu(OtBu) in step 6 is not used. In addition, homocysteine is used instead of cysteine in step 7. Finally, Wang resin is used instead of Rink resin.

### EXAMPLE 143

### Synthesis of Compound No. 160: Ac-nLeu-cyclo[hCys-His-D-Phe-Arg-Trp-Cys]-NH₂

Can be prepared according to Example 1, with the exception that Fmoc-Glu(OtBu) in step 6 is not used. Fmoc-Cys(Trt) in step 7 and Fmoc-Arg(pbf) in step 8 are replaced with Fmoc-hCys(Trt) and Fmoc-nLeu, respectively.

### EXAMPLE 144

### Synthesis of Compound No. 161: N-phenylsulfonyl-Gly-cyclo[hCys-His-D-Phe-Arg-Trp-Cys]-NH₂

Can be prepared according to Example 1, with the exception that Fmoc-Glu(OtBu) in step 6 is not used. In addition, Fmoc-hCys(Trt) and Fmoc-Gly are used in steps 7 and 8 instead of Fmoc-Cys(Trt) and Fmoc-Arg(pbf), respectively. Acetic anhydride in step 9 is replaced with phenylsulfonylchloride.

### EXAMPLE 145

### Synthesis of Compound No. 162: Tyr-Arg-cyclo[hCys-His-D-Phe-Arg-Trp-Cys]-NH₂

Can be prepared according to Example 1, with the exception that Fmoc-Glu(OtBu) and acetylation with acetic anhydride in steps 6 and 9, respectively, are not used. In addition, Fmoc-Tyr(tBu) is added between steps 8 and 9. Finally, homocysteine is used instead of cysteine in step 7.

### EXAMPLE 146

### Synthesis of Compound No. 163: Tyr-Arg-cyclo[hCys-His-D-Phe-Arg-Trp-Cys]-OH

Can be prepared according to Example 1, with the exception that acetylation with acetic anhydride in step 9 is not used, and homocysteine is used instead of cysteine in step 7. In addition, Fmoc-Tyr(tBu) is added after step 8. Finally, Wang resin is used instead of Rink resin.

### EXAMPLE 147

### Synthesis of Compound No. 164: Ac-Tyr-Arg-cyclo[hCys-His-D-Phe-Arg-Trp-Cys]-NH₂

Can be prepared according to Example 1, with the exception that homocysteine is used instead of cysteine in step 7. Fmoc-Glu(OtBu) is not used in step 6. Fmoc-Tyr(tBu) is added between steps 8 and 9.

### EXAMPLE 148

### Synthesis of Compound No. 165: Ac-Tyr-Arg-cyclo[hCys-His-D-Phe-Arg-Trp-Cys]-OH

Can be prepared according to Example 1, with the exception that Fmoc-Glu(OtBu) is not used. In addition, homocysteine is used instead of cysteine in step 7. Fmoc-Tyr(tBu) is added after step 8. Finally, Wang resin is used instead of Rink resin.

### EXAMPLE 149

### Synthesis of Compound No. 166: Ac-Tyr-Arg-cyclo[hCys-Glu-His-D-Phe-Arg-Trp-Cys]-NH₂

Can be prepared according to Example 1, with the exception that Fmoc-Tyr(tBu) is used between steps 8 and 9. Homocysteine is used instead of cysteine in step 7.

### EXAMPLE 150

### Synthesis of Compound No. 167: Ac-cyclo[hCys-His-(β-cyclohexyl-D-Ala)-Arg-Trp-Cys]-NH₂

Can be prepared according to Example 1, with the exception that Fmoc-Glu(OtBu) in step 6 and Fmoc-Arg(pbf) in step 8 are not used. In addition, Fmoc-hCys(Trt) and Fmoc-(β-cyclohexyl-D-Ala) are used instead of Fmoc-Cys(Trt) in step 7 and Fmoc-D-Phe in step 4, respectively.

### EXAMPLE 151

### Synthesis of Compound No. 168: Ac-cyclo[hCys-His-D-Phe-Arg-Trp-penicillamine]-NH₂

Can be prepared according to Example 1, with the exception that Fmoc-Glu(OtBu) in step 6 and Fmoc-Arg(pbf) in step 8 are not used. In addition, Fmoc-penicillamine(Trt) and Fmoc-hCys(Trt) are used instead of Fmoc-Cys(Trt) in steps 1 and 7, respectively.

### EXAMPLE 152

### Synthesis of Compound No. 169: Ac-cyclo[hCys-His-(4-Cl-D-Phe)-Arg-Trp-penicillamine]-NH₂

Can be prepared according to Example 1, with the exception that Fmoc-Glu(OtBu) in step 6 and Fmoc-Arg(pbf) in step 8 are not used. Fmoc-Cys(Trt) in steps 1 and 7, and Fmoc-D-Phe in step 4, are replaced with Fmoc-penicillamine(Trt), Fmoc-hCys(Trt), and Fmoc-4-Cl-D-Phe, respectively.

### EXAMPLE 153

### Synthesis of Compound No. 170: N-hexanoyl-cyclo[hCys-His-D-Phe-Arg-Trp-penicillamine]-NH₂

Can be prepared according to Example 1, with the exception that Fmoc-Glu(OtBu) in step 6 and Fmoc-Arg(pbf) in step 8 are not used. Fmoc-hCys(Trt) and Fmoc-penicillamine(Trt) are used in steps 7 and 1, respectively, instead of Fmoc-Cys(Trt). In addition, in step 9, acetic anhydride is replaced with n-hexanoic acid, which is pre-activated with DIC (1,3-diisopropylcarbodiimide)/HOBt (1-hydroxylbenzo-triazole).

### EXAMPLE 154

### Synthesis of Compound No. 171: N-cyclopentanecarbonyl-cyclo[hCys-His-D-Phe-Arg-Trp-penicillamine]-NH₂

Can be prepared according to Example 1, with the exception that Fmoc-Glu(OtBu) in step 6 and Fmoc-Arg(pbf) in step 8 are not used. Fmoc-Cys(Trt) in steps 1 and 7 are replaced with Fmoc-penicillamine(Trt) and Fmoc-hCys(Trt), respectively. In addition, in step 9, acetic acid anhydride is replaced with cyclopentane carboxylic acid, which is pre-activated with DIC (1,3-diisopropylcarbodiimide)/HOBt (1-hydroxyl-benzotriazole).

### EXAMPLE 155

### Synthesis of Compound No. 172: N-cyclohexanecarbonyl-cyclo[hCys-His-D-Phe-Arg-Try-penicillamine]-NH₂

Can be prepared according to Example 1, with the exception that Fmoc-Glu(OtBu) in step 6 and Fmoc-Arg(pbf) in step 8 are not used. Fmoc-hCys(Trt) and Fmoc-penicillamine(Trt) are used in steps 7 and 1, respectively, instead of Fmoc-Cys(Trt). In addition, in step 9, acetic anhydride is replaced with cyclohexane carboxylic acid, which is pre-activated with DIC (1,3-diisopropylcarbodiimide)/HOBt (1-hydroxyl-benzotriazole).

### EXAMPLE 156

### Synthesis of Compound No. 173: N-benzoyl-cyclo[hCys-His-D-Phe-Arg-Trp-penicillamine]-NH₂

Can be prepared according to Example 1, with the exception that Fmoc-Glu(OtBu) in step 6 and Fmoc-Arg(pbf) in step 8 are not used. Fmoc-hCys(Trt) and Fmoc-penicillamine(Trt) are used in steps 7 and 1, respectively, instead of Fmoc-Cys(Trt). In addition, in step 9, acetic anhydride is replaced with benzoic acid, which is pre-activated with DIC (1,3-diisopropylcarbodiimide)/HOBt (1-hydroxylbenzotriazole).

### EXAMPLE 157

### Synthesis of Compound No. 174: 4-phenylbutyryl-cyclo[hCys-His-D-Phe-Arg-Trp-penicillamine]-NH₂

Can be prepared according to Example 1, with the exception that Fmoc-Glu(OtBu) in step 6 and Fmoc-Arg(pbf) in step 8 are not used. Fmoc-hCys(Trt) and Fmoc-penicillamine(Trt) are used in steps 7 and 1, respectively, instead of Fmoc-Cys(Trt). In addition, in step 9, acetic anhydride is replaced with 4-phenylbutyric acid, which is pre-activated with DIC (1,3-diisopropylcarbodiimide)/HOBt (1-hydroxyl-benzotriazole).

### EXAMPLE 158

### Synthesis of Compound No. 175: N-(phenylsulfonyl)-cyclo[hCys-His-D-Phe-Arg-Trp-penicillamine]-NH₂

Can be prepared according to Example 1, with the exception that Fmoc-Glu(OtBu) in step 6 and Fmoc-Arg(pbf) in step 8 are not used. In addition, Fmoc-hCys(Trt) and Fmoc-penicillamine(Trt) are used in steps 7 and 1, respectively, instead of Fmoc-Cys(Trt). Acetic anhydride in step 9 is replaced with phenylsulfonylchloride.

### EXAMPLE 159

### Synthesis of Compound No. 176: (4-benzenesulfonamide)butyryl-cyclo[hCys-His-D-Phe-Arg-Trp-penicillamine]-NH₂

Can be prepared according to Example 1, with the exception that Fmoc-Glu(OtBu) in step 6 is not used. In step 8, Fmoc-Arg(pbf) is replaced with Fmoc-γ-amino-butyric acid. In addition, Fmoc-hCys(Trt) and Fmoc-penicillamine(Trt) are used in steps 7 and 1, respectively, instead of Fmoc-Cys(Trt). Acetic anhydride in step 9 is replaced with phenylsulfonylchloride.

### EXAMPLE 160

### Synthesis of Compound No. 177: Ac-nLeu-cyclo[hCys-His-D-Phe-Arg-Trp-penicillamine]-NH₂

Can be prepared according to Example 1, with the exception that Fmoc-Glu(OtBu) in step 6 is not used. Fmoc-Cys(Trt) in steps 1 and 7, and Fmoc-Arg(pbf) in step 8, are replaced with Fmoc-penicillamine(Trt), Fmoc-hCys(Trt) and Fmoc-nLeu, respectively.

### EXAMPLE 161

### Synthesis of Compound No. 178: N-phenylsulfonyl-Gly-cyclo[hCys-His-D-Phe-Arg-Trp-penicillamine]-NH₂

Can be prepared according to Example 1, with the exception that Fmoc-Glu(OtBu) in step 6 is not used. In addition, Fmoc-penicillamine(Trt), Fmoc-hCys(Trt) and Fmoc-Gly are used in steps 1, 7, and 8 instead of Fmoc-Cys(Trt), Fmoc-Cys(Trt), and Fmoc-Arg(pbf), respectively. Acetic anhydride in step 9 is replaced with phenylsulfonyl-chloride.

### EXAMPLE 162

### Synthesis of Compound No. 179: cyclo[3-thiopropionyl-His-D-Phe-Arg-Trp-hCys]-NH₂

Can be prepared according to Example 1, with the exception that Fmoc-Glu(OtBu) in step 6, Fmoc-Arg(pbf) in step 8, and acetylation with acetic anhydride in step 9 are not used. In addition, Fmoc-hCys(Trt) and (S-Trt)-3-thiopropionic acid are used instead of Fmoc-Cys(Trt) in steps 1 and 7, respectively.

### EXAMPLE 163

### Synthesis of Compound No. 180: cyclo[Cys-His-D-Phe-Arg-Trp-hCys]-NH₂

Can be prepared according to Example 1, with the exception that Fmoc-Glu(OtBu) and Fmoc-Arg(pbf) in steps 6 and 8 are not used. Acetylation with acetic anhydride in step 9 is not used. In addition, Fmoc-hCys(Trt) is used instead of Fmoc-Cys(Trt) in step 1.

### EXAMPLE 164

### Synthesis of Compound No. 181: cyclo[Cys-His-(4-F-D-Phe)-Arg-Trp-hCys]-NH₂

Can be prepared according to Example 1, with the exception that Fmoc-Glu(OtBu) in step 6, Fmoc-Arg(pbf) in step 8, and acetylation with acetic anhydride in step 9 are not used. In addition, Fmoc-hCys(Trt) and Fmoc-(4-F-D-Phe) are used instead of Fmoc-Cys(Trt) in step 1 and Fmoc-D-Phe in step 4, respectively.

### EXAMPLE 165

### Synthesis of Compound No. 182: cyclo[Cys-His-(4-Cl-D-Phe)-Arg-Trp-hCys]-NH₂

Can be prepared according to Example 1, with the exception that Fmoc-Glu(OtBu) and Fmoc-Arg(pbf) in steps 6 and 8 are not used. Acetylation with acetic anhydride in step 9 is not used. In addition, Fmoc-hCys(Trt) and Fmoc-4-Cl-D-Phe are used instead of Fmoc-Cys(Trt) and Fmoc-D-Phe, respectively, in steps 1 and 4.

### EXAMPLE 166

### Synthesis of Compound No. 183: Ac-cyclo[Cys-His-(4-Cl-D-Phe)-Arg-Trp-hCys]-NH₂

Can be prepared according to Example 1, with the exception that Fmoc-Glu(OtBu) and Fmoc-Arg(pbf) in steps 6 and 8 are not used. In addition, Fmoc-hCys(Trt) and Fmoc-4-Cl-D-Phe are used instead of Fmoc-Cys(Trt) and Fmoc-D-Phe, respectively, in steps 1 and 4.

### EXAMPLE 167

### Synthesis of Compound No. 184: Ac-cyclo[Cys-His-(4-F-D-Phe)-Arg-Trp-hCys]-NH₂

Can be prepared according to Example 1, with the exception that Fmoc-Glu(OtBu) in step 6 and Fmoc-Arg(pbf) in step 8 are not used. In addition, Fmoc-hCys(Trt) and Fmoc-4-F-D-Phe are used instead of Fmoc-Cys(Trt) in step 1 and Fmoc-D-Phe in step 4, respectively.

### EXAMPLE 168

### Synthesis of Compound No. 185: Ac-cyclo[Cys-His-(4-Cl-D-Phe)-Arg-Trp-hCys]-NH₂

Can be prepared according to Example 1, with the exception that Fmoc-Glu(OtBu) in step 6 and Fmoc-Arg(pbf) in step 8 are not used. In addition, Fmoc-hCys(Trt) and Fmoc-4-Cl-D-Phe are used instead of Fmoc-Cys(Trt) in step 1 and Fmoc-D-Phe in step 4, respectively.

### EXAMPLE 169

### Synthesis of Compound No. 186: Arg-cyclo[Cys-His-D-Phe-Arg-Trp-hCys]-NH₂

Can be prepared according to Example 1, with the exception that Fmoc-Glu(OtBu) in step 6 and acetylation with acetic anhydride in step 9 are not used. In addition, Fmoc-hCys(Trt) is used instead of Fmoc-Cys(Trt) in step 1.

### EXAMPLE 170

### Synthesis of Compound No. 187: Arg-cyclo[Cys-His-(4-F-D-Phe)-Arg-Trp-hCys]-NH₂

Can be prepared according to Example 1, with the exception that Fmoc-Glu(OtBu) in step 6 and acetylation with acetic anhydride in step 9 are not used. In addition, Fmoc-hCys(Trt) and Fmoc-4-F-D-Phe are used instead of Fmoc-Cys(Trt) in step 1 and Fmoc-D-Phe in step 4, respectively.

### EXAMPLE 171

### Synthesis of Compound No. 188: Arg-cyclo[Cys-His-(4-Cl-D-Phe)-Arg-Trp-hCys]NH₂

Can be prepared according to Example 1, with the exception that Fmoc-Glu(OtBu) in step 6 and acetylation with acetic anhydride in step 9 are not used. In addition, Fmoc-hCys(Trt) and Fmoc-4-Cl-D-Phe are used instead of Fmoc-Cys(Trt) and Fmoc-D-Phe, respectively, in steps 1 and 4.

### EXAMPLE 172

### Synthesis of Compound No. 189: Ac-Arg-cyclo[Cys-His-D-Phe-Arg-Trp-hCys]-NH₂

Can be prepared according to Example 1, with the exception that Fmoc-Glu(OtBu) in step 6 is not used. In addition, Fmoc-hCys(Trt) is used instead of Fmoc-Cys(Trt) in step 1.

### EXAMPLE 173

### Synthesis of Compound No. 190: Ac-Arg-cyclo[Cys-His-(4-F-D-Phe)-Arg-Trp-hCys]-NH₂

Can be prepared according to Example 1, with the exception that Fmoc-Glu(OtBu) in step 6 is not used. In addition, Fmoc-hCys(Trt) and Fmoc-4-F-D-Phe are used instead of Fmoc-Cys(Trt) in step 1 and Fmoc-D-Phe in step 4, respectively.

### EXAMPLE 174

### Synthesis of Compound No. 191: Ac-Arg-cyclo[Cys-His-(4-Cl-D-Phe)-Ar-g-Trp-hCys]-NH₂

Can be prepared according to Example 1, with the exception that Fmoc-Glu(OtBu) in step 6 is not used. In addition, Fmoc-hCys(Trt) and Fmoc-4-Cl-D-Phe are used instead of Fmoc-Cys(Trt) in step 1 and Fmoc-D-Phe in step 4, respectively.

### EXAMPLE 175

### Synthesis of Compound No. 192: Ac-Tyr-Arg-cyclo[Cys-Glu-His-D-Phe-Arg-Trp-hCys]-NH₂

Can be prepared according to Example 1, with the exception that Fmoc-hCys(Trt) is used instead of Fmoc-Cys(Trt) in step 1. Fmoc-Tyr(tBu) is added between steps 8 and 9.

### EXAMPLE 176

### Synthesis of Compound No. 193: Ac-cyclo[hCys-His-D-Phe-Arg-Trp-hCys]-NH₂

Can be prepared according to Example 1, with the exception that Fmoc-hCys(Trt) is used instead of Fmoc-Cys(Trt) in steps 1 and 7. Fmoc-Glu(OtBu) is not used in step 6. Fmoc-Arg(Pbf) is not used in step 8.

### EXAMPLE 177

### Synthesis of Compound No. 194: Arg-cyclo[hCys-His-D-Phe-Arg-Trp-hCys]-NH₂

Can be prepared according to Example 1, with the exception that Fmoc-hCys(Trt) is used instead of Fmoc-Cys(Trt) in steps 1 and 7. Fmoc-Glu(OtBu) is not used in step 6. Acetylation with acetic anhydride in step 9 is not used.

### EXAMPLE 178

### Synthesis of Compound No. 195: Ac-Arg-cyclo[hCys-His-D-Phe-Arg-Trp-hCys]-NH₂

Can be prepared according to Example 1, with the exception that Fmoc-hCys(Trt) is used instead of Fmoc-Cys(Trt) in steps 1 and 7. Fmoc-Glu(OtBu) is not used in step 6.

### EXAMPLE 179

### Synthesis of Compound No. 196: Ac-Tyr-Arg-cyclo[hCys-His-D-Phe-Arg-Trp-hCys]-NH₂

Can be prepared according to Example 1, with the exception that Fmoc-hCys(Trt) is used instead of Fmoc-Cys(Trt) in steps 1 and 7. Fmoc-Glu(OtBu) is not used in step 6. Fmoc-Tyr(tBu) is added between steps 8 and 9.

### EXAMPLE 180

### Synthesis of Compound No. 197: Ac-Tyr-Arg-cyclo[hCys-Glu-His-D-Phe-Arg-Trp-hCys]-NH₂

Can be prepared according to Example 1, with the exception that Fmoc-hCys(Trt) is used instead of Fmoc-Cys(Trt) in steps 1 and 7. Fmoc-Tyr(tBu) is added between steps 8 and 9.

### EXAMPLE 181

### Synthesis of Compound No. 198: Ac-cyclo(s-CH2)-S)[Cys-His-D-Phe-Arg-Trp-Cys]-NH₂

Can be prepared according to Example 1, with the exception that Fmoc-Glu(OtBu) in step 6 and Fmoc-Arg(pbf) in step 8 are not used. In addition, after the cleavage and deprotection of the linear peptide from the resin, the cyclization to form the disulfide bond is not carried out. Instead, the crude peptide (200 mg) is suspended in 200 mL of dichloromethane/acetonitrile (1 :1 v/v) containing 3 mL of 1.0 M TBAF (tetrabutyl ammonium fluoride in THF) and stirring at room temperature for 30 min. Then, 3 mL of glacial acetic acid is added to quench the reaction. The solvents are removed under vacuum.

### EXAMPLE 182

### Synthesis of Compound No. 83: Ac-Tyr-Arg-cyclo[Cys-Glu-His-(4-F-D-Phe)-Arg-Trp-Cys]-NH₂

The side-chain protection scheme of amino acids is consistent with standard t-butyloxycarbonyl tBoc chemistry, as shown in Scheme B below: Cys(4-MeBzl), Trp(CHO), 4-F-D-Phe, His(3-bom), Glu(O-cHx), Cys(4-MeBzl), Arg(p-Tos), Tyr(2-BrZ). Commercially available MBHA resin (Midwest Biotech) is utilized as the solid support. The couplings are carried out either manually by single coupling each residue with a three-fold excess of amino acid activated with DCC/HOBt or by automated methods using an ABI 431A or ABI 433A synthesizer programmed with the manufacturer's standard t-Boc protocol. N-terminal acetylation is accomplished with 5 equivalents acetic anhydride, 10 equivalents DIEA in dry DMF, 1 hour at room temperature. The tryptophan formyl group is deprotected by treatment of the resin-bound peptide with 20% piperidine in DMF, followed by washing with DMF and dichloromethane. The peptides are simultaneously cleaved from the resin and deprotected by treatment with liquid hydrogen fluoride at 0° C. for 1 hour in the presence m-cresol and thiocresol scavengers. The peptides are recovered by ether precipitation, washed with ether, extracted into aqueous acetic acid, and lyophilized.

Cyclization Protocol

The oxidation of the free cysteine sulfhydryl groups is accomplished either by air oxidation in 0.2 M ammonium acetate buffer containing 20% dimethyl sulfoxide (DMSO) at pH 7.0, or by treatment with 2,2′-pyridyldisulfide in 2.7 M guanidine buffer containing 30% DMSO. In each case, the final product is isolated by high performance liquid chromatography.

Purification

Purification is accomplished using standard preparative HPLC techniques. Immediately following the cyclization, the peptide is diluted and loaded onto an HPLC column and eluted with an aqueous 0.1% trifluoroacetic acid/acetonitrile gradient while monitoring at 214 nm. The appropriate fractions are pooled and lyophilized. Further characterization of the final product is performed using analytical HPLC and mass spectral analysis.

Conversion to Acetate Salt

The peptide is by adsorbed onto a 2.1×25 cm Zorbax C18 preparative column, which is equilibrated with 0.1% TFA/H₂O. The column is then washed with 2 volumes of 0.1 M ammonium acetate/5% acetonitrile followed by 2 column volumes of water. The peptide is eluted using 2% acetic acid and lyophilized.

The product is characterized using mass spectrometry and HPLC purity detected using acceptable methods in the art and is summarized in Table 2 below.

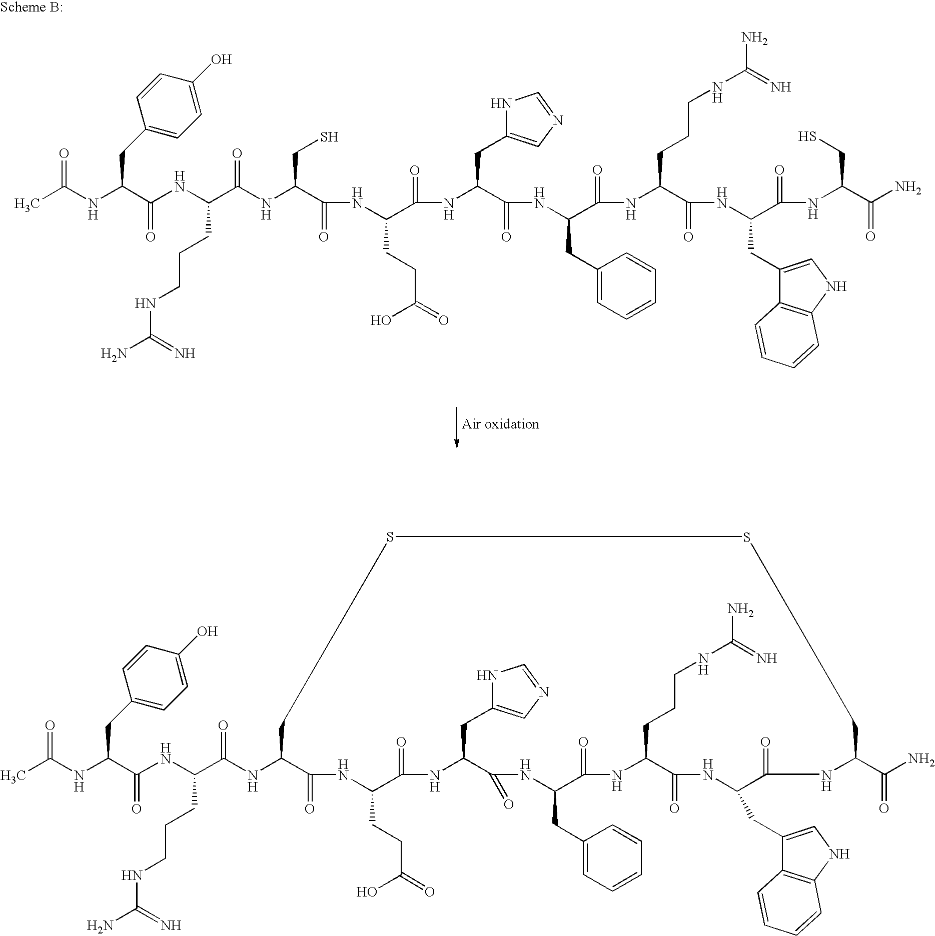

### EXAMPLE 183

### Synthesis of Compound No. 97: Ac-Tyr-Arg-cyclo[Cys-Glu-(5-Me-His)-D-Phe-Arg-Trp-Cys]-NH₂ and Synthesis of Compound No. 98: Ac-Tyr-Arg-cyclo[Cys-Glu-(5-Me-D-His)-D-Phe-Arg-Trp-Cys]-NH₂

Can be prepared according to Example 182, with the exception that Boc-5-Me-(D/L)-His(3-Boc) is used in step 5 instead of Boc-His(3-Bom). The two peptide-isomers are easily separated on HPLC, which affords:
Ac-Tyr-Arg-cyclo[Cys-Glu-(5-Me-His)-D-Phe-Arg-Trp-Cys]-NH₂ and Ac-Tyr-Arg-cyclo[Cys-Glu-(5-Me-D-His)-D-Phe-Arg-Trp-Cys]-NH₂.

The absolute configurations of the 5-Me-His residue in each peptide are defined by two-dimensional NMR techniques with proper standard peptides and controls.

### EXAMPLE 184

### Synthesis of Compound No. 102: Ac-Tyr-Arg-cyclo[Cys-Glu-(1-pyrazolyl-Ala)-D-Phe-Arg-Trp-Cys]-NH₂ and Synthesis of Ac-Tyr-Arg-cyclo[Cys-Glu-(1-pyrazolyl-D-Ala)-D-Phe-Arg-Trp-Cys]-NH₂

Can be prepared according to Example 182, with the exception that Boc-1-Pyrazolyl-(D/L)Ala is used in step 5 instead of Boc-His(3-Bom). The two peptide-isomers are easily separated on HPLC, which affords:
Ac-Tyr-Arg-cyclo[Cys-Glu-(1-pyrazolyl-D-Ala)-D-Phe-Arg-Trp-Cys]-NH₂ and Ac-Tyr-Arg-cyclo[Cys-Glu-(1-pyrazolyl-Ala)-D-Phe-Arg-Trp-Cys]-NH₂

The absolute configurations of this His residue replacement in each peptide are defined by two-dimensional NMR techniques with proper standard peptides and controls.

### EXAMPLE 185

### Synthesis of Compound No. 103: Ac-Tyr-Arg-cyclo[Cys-Glu-(4-phenyl-1H-imidazol-2-yl-Ala)-D-Phe-Arg-Trp-Cys]-NH₂ and Synthesis of Compound No. 104: Ac-Tyr-Arg-cyclo[Cys-Glu-(4-phenyl-1H-imidazol-2-yl-D-Ala)-D-Phe-Arg-Trp-Cys]-NH₂

Can be prepared according to Example 182, with the exception that Boc-4-phenyl-1H-imidazolyl-(D/L)Ala is used in step 5 instead of Boc-His(3-Bom). The two peptide-isomers are easily separated on HPLC, which affords:
Ac-Tyr-Arg-cyclo[Cys-Glu-(4-phenyl-1H-imidazol-2-yl-D-Ala)-D-Phe-Arg-Trp-Cys]-NH₂ Ac-Tyr-Arg-cyclo[Cys-Glu-(4-phenyl-1H-imidazol-2-yl-Ala)-D-Phe-Arg-Trp-Cys]-NH₂

The absolute configurations of this His residue replacement in each peptide are defined by two-dimensional NMR techniques with proper standard peptides and controls.

### EXAMPLE 186

### Synthesis of Compound No. 105: Ac-Tyr-Arg-cyclo[Cys-Glu-(2-pyrazine-Ala)-D-Phe-Arg-Trp-Cys]-NH₉ and Synthesis of Ac-Tyr-Arg-cyclo[Cys-Glu-(2-pyrazine-D-Ala)-D-Phe-Arg-Trp-Cys]-NH₂

Can be prepared according to Example 182, with the exception that Boc-2-Pyrazine-(D/L)Ala is used in step 5 instead of Boc-His(3-Bom). The two peptide-isomers are easily separated on HPLC, which affords:
Ac-Tyr-Arg-cyclo[Cys-Glu-(2-pyrazine-D-Ala)-D-Phe-Arg-Trp-Cys]-NH₂ and Ac-Tyr-Arg-cyclo[Cys-Glu-(2-pyrazine-Ala)-D-Phe-Arg-Trp-Cys]-NH₂

The absolute configurations of this His residue replacement in each peptide are defined by two-dimensional NMR techniques with proper standard peptides and controls.

### EXAMPLE 187

### Synthesis of Compound No. 106: Ac-Tyr-Arg-cyclo[Cys-Glu-(β-(1,2,4-triazol-3-yl)-Ala)-D-Phe-Arg-Trp-Cys]-NH₂, Synthesis of Compound No. 107: Ac-Tyr-Arg-cyclo[Cys-Glu-(β-(1,2,4-triazol-3-yl)-D-Ala)-D-Phe-Arg-Trp-Cys]-NH₂, Synthesis of Compound No. 108: Ac-Tyr-Arg-cyclo[Cys-Glu-(β-((1-benzyl)-1,2,4-triazol-3-yl)-Ala)-D-Phe-Arg-TrM-Cys]-NH₂ and Synthesis of Compound No. 109: Ac-Tyr-Arg-cyclo[Cys-Glu-(β-((1-benzyl)-1,2,4-triazol-3-yl)-D-Ala)-D-Phe-Arg-Trp-Cys]-NH₂

Can be prepared according to Example 182, with the exception that Boc-(β-((1-benzyl)-1,2,4-triazol-3-yl)-(D/L)Ala is used in step 5 instead of Boc-His(3-Bom). During HF cleavage, the benzyl protecting-group is partially removed, and the synthesis yields four peptide-isomers. The four peptide-isomers are easily separated on HPLC, which affords:
Ac-Tyr-Arg-cyclo[Cys-Glu-(β-((1-benzyl)-1,2,4-triazol-3-yl)-D-Ala)-D-Phe-Arg-Trp-Cys]-NH₂, Ac-Tyr-Arg-cyclo[Cys-Glu-(β-(1,2,4-triazol-3-yl)-D-Ala)-D-Phe-Arg-Trp-Cys]-NH₂, Ac-Tyr-Arg-cyclo[Cys-Glu-(β-((1-benzyl)-1,2,4-triazol-3-yl)-Ala)-D-Phe-Arg-Trp-Cys]-NH₂, and Ac-Tyr-Arg-cyclo[Cys-Glu-(β-(1,2,4-triazol-3-yl)-Ala)-D-Phe-Arg-Trp-Cys]-NH₂.

The absolute configurations of this histidine residue replacement in each peptide are defined by two-dimensional NMR techniques with proper peptide standards and controls.

TABLE 2
Analytical Data

| | Theoretical MW | Observed MW | |
|---|---|---|---|
| Compound No. | (Daltons) | (Daltons) | HPLC purity (%) |
| 1 | 890.06 | 889.8 | 91.2 |
| 2 | 1268.47 | 1268.6 | 99.3 |
| 3 | 1280.5 | 1280.16 | 98.4 |
| 4 | 1365.6 | 1364.84 | 99.1 |
| 5 | 1323.5 | 1322.73 | 99.4 |
| 6 | 1005.2 | | |
| 7 | 1324.52 | 1324.07 | 95.8 |
| 8 | 1018.2 | | |
| 9 | 1338.5 | | 95.0 |
| 10 | 1352.6 | | 99.0 |
| 11 | 1224.4 | | >95 |
| 12 | 1266.49 | 1266.21 | 98.6 |
| 13 | 1346.58 | 1345.67 | 99.1 |
| 14 | 1322.6 | 1322.53 | 98.8 |
| 15 | 1003.2 | | |
| 16 | 1018.2 | | |
| 17 | 1311.5 | | |
| 18 | 1340.63 | 1340.14 | 97.6 |
| 19 | 1356.62 | 1356.56 | 86.0 |
| 20 | 1306.56 | 1306.28 | 97.5 |
| 21 | 1296.52 | 1296.02 | 98.1 |
| 22 | 1310.55 | 1310.15 | 98.2 |
| 23 | 1395.65 | 1395.02 | 98.0 |
| 24 | 1372.6 | 1372.9 | 94.6 |
| 25 | 1308.57 | 1308.27 | 98.6 |
| 26 | 1197.39 | | |
| 27 | 1197.39 | | |
| 28 | 1360.56 | 1360.2 | 95.7 |
| 29 | 977.1 | | 99.0 |
| 30 | 1019.2 | | |
| 31 | 1037.2 | | |
| 32 | 1053.6 | | |
| 33 | 1098.1 | | |
| 34 | 1033.21 | 1033.22 | 97.5 |
| 35 | 1244.5 | 1244.4 | 90.3 |
| 36 | 1203.4 | | >95 |
| 37 | 1033.2 | | |
| 38 | 1047.2 | | |
| 39 | 1061.2 | | |
| 40 | 1090.26 | 1089.96 | 90.2 |
| 41 | 1104.3 | | |
| 42 | 1132.34 | 1132.47 | 97.3 |
| 43 | 1105.3 | | |
| 44 | 1119.3 | | |
| 45 | 1134.3 | | 99.0 |
| 46 | 1133.32 | 1132.7 | 96.6 |
| 47 | 1175.37 | 1175.2 | 98.6 |
| 48 | 1175.4 | | |
| 49 | 1176.4 | | 99.0 |
| 50 | 1209.8 | | >95 |
| 51 | 1189.4 | 1189.56 | 98.7 |
| 52 | 1175.40 | 1175.4 | 98.0 |
| 53 | 1176.35 | | |
| 54 | 1189.4 | | |
| 55 | 1176.3 | | |
| 56 | 1190.38 | 1190.35 | 96.2 |
| 57 | 1132.3 | | |
| 58 | 1147.3 | | |
| 59 | 1189.4 | | |
| 60 | 1132.3 | | |
| 61 | 1316.5 | | >95 |
| 62 | 1133.3 | | |
| 63 | 1118.3 | | |
| 64 | 1323.55 | 1323.3 | 94.7 |
| 65 | 1422.64 | 1422.8 | 92.9 |
| 66 | 1281.5 | | |
| 67 | 1307.5 | | |
| 68 | 1296.48 | | |
| 69 | 1297.49 | 1297.29 | 96.1 |
| 70 | 1395.7 | | 90.0 |
| 71 | 1425.62 | 1425.69 | 97.9 |
| 72 | 1338.54 | | |
| 73 | 1339.53 | 1339.34 | 96.7 |
| 74 | 1396.6 | | |
| 75 | 1416.6 | | |
| 76 | 1411.59 | 1141.51 | 97.3 |
| 77 | 1474.6 | | |
| 78 | 1325.5 | | >95.0 |
| 79 | 1338.55 | 1338.52 | 96.6 |
| 80 | 1338.5 | | |
| 81 | 1352.6 | | >94.0 |
| 82 | 1352.6 | 1356.2 | 88.3 |
| 83 | 1356.54 | 1355.95 | 96.0 |
| 84 | 1370.56 | 1370.27 | 96.5 |
| 85 | 1370.56 | 1369.85 | 99.8 |
| 86 | 1372.99 | 1372.19 | 95.5 |
| 87 | 1387.02 | 1387.1 | 95.0 |
| 88 | 1387.02 | 1386.50 | 94.4 |
| 89 | 1417.4 | | 92.0 |
| 90 | 1431.47 | 1431.1 | 97.0 |
| 91 | 1431.47 | 1431.91 | 95.0 |
| 92 | 1352.57 | 1352.16 | 95.8 |
| 93 | 1368.57 | 1368.27 | 96.9 |
| 94 | 1382.6 | 1382.86 | 97.8 |
| 95 | 1382.60 | 1382.40 | 98.6 |
| 96 | 1352.57 | 1352.15 | 96.1 |
| 97 | 1352.57 | 1352.1 | 92.9 |
| 98 | 1352.57 | 1352.2 | 99.2 |
| 99 | 1428.67 | 1428.48 | 97.0 |
| 100 | 1428.67 | 1428.54 | 96.6 |
| 101 | 1458.7 | 1458.5 | 99.4 |
| 102 | 1338.55 | 1338.2 | 95.0 |
| 103 | 1414.64 | 1414.1 | 95.0 |
| 104 | 1414.64 | 1413.7 | 95.0 |
| 105 | 1350.56 | 1349.8 | 95.0 |
| 106 | 1339.53 | 1338.6 | 97.4 |
| 107 | 1339.53 | 1338.8 | 99.2 |
| 108 | 1429.66 | 1429.1 | 96.7 |
| 109 | 1429.66 | 1429.4 | 89.5 |
| 110 | 1338.54 | 1338.49 | 96.4 |
| 111 | 1354.61 | 1354.10 | 96.5 |
| 112 | 1355.60 | 1355.51 | 94.2 |
| 113 | 1349.57 | 1349.08 | 89.9 |
| 114 | 1382.6 | | >95 |
| 115 | 1426.6 | | >95 |
| 116 | 1412.6 | | >95 |
| 117 | 1437.7 | | 90.0 |
| 118 | 1467.66 | 1467.24 | 97.6 |
| 119 | 1522.4 | | >95 |
| 120 | 1509.7 | | >95 |
| 121 | 1563.8 | 1563.1 | 99.9 |
| 122 | 1550.8 | | >95 |
| 123 | 1641.9 | 1641.8 | 98.1 |
| 124 | 1339.53 | 1339.2 | 94.9 |
| 125 | 1353.56 | 1353.5 | 94.3 |
| 126 | 1352.57 | 1351.66 | 93.3 |
| 127 | 1352.57 | 1352.58 | 87.0 |
| 128 | 1310.5 | | |
| 129 | 1271.5 | 1271.4 | 98.0 |
| 130 | 1281.5 | | |
| 131 | 1397.57 | 1397.2 | 96.8 |
| 132 | 862.05 | 862.2 | 98.4 |
| 133 | 863.04 | 862.95 | 94.9 |
| 134 | 880.04 | 880.6 | 99.4 |
| 135 | 896.50 | 896.2 | 98.6 |
| 136 | 904.09 | 903.9 | 99.8 |
| 137 | 904.09 | 904.2 | 99.3 |
| 138 | 905.08 | 905.15 | 98.9 |
| 139 | 922.08 | 922.6 | 99.0 |
| 140 | 938.54 | 938.2 | 96.2 |
| 141 | 930.13 | 930.0 | 99.7 |
| 142 | 944.15 | 943.6 | 99.5 |
| 143 | 958.18 | 958.0 | 99.0 |
| 144 | 972.20 | 971.6 | 99.0 |
| 145 | 960.19 | 959.6 | 99.0 |
| 146 | 966.16 | 965.5 | 99.0 |
| 147 | 1008.24 | 1007.8 | 99.0 |
| 148 | 975.17 | 974.6 | 96.5 |
| 149 | 1003.22 | 1002.8 | 99.1 |
| 150 | 1002.21 | 1002.4 | >99 |
| 151 | 1052.27 | 1052.3 | 95.8 |
| 152 | 1101.30 | 1100.8 | 98.8 |
| 153 | 1018.24 | 1018.1 | 97.9 |
| 154 | | | |
| 155 | 1019.23 | 1019.01 | 97.0 |
| 156 | 1032.27 | 1032.4 | 79.9 |
| 157 | 1032.27 | 1032.4 | 95.9 |
| 158 | 1060.28 | 1060.31 | 98.4 |
| 159 | 1061.26 | 1061.19 | 97.7 |
| 160 | 1017.25 | 1017.0 | 99.0 |
| 161 | 1059.26 | 1058.6 | 99.5 |
| 162 | 1181.42 | 1181.3 | 97.6 |
| 163 | 1182.4 | 1182.32 | 94.7 |
| 164 | 1223.46 | 1222.89 | 98.1 |
| 165 | 1224.44 | 1224.47 | 98.9 |
| 166 | 1352.6 | | |
| 167 | 910.14 | 910.2 | 97.8 |
| 168 | 932.14 | 931.6 | 97.3 |
| 169 | 966.59 | 966.2 | 93.5 |
| 170 | 988.25 | 987.6 | 99.0 |
| 171 | 986.24 | 986.0 | 99.7 |
| 172 | 1000.26 | 999.6 | 99.0 |
| 173 | 994.21 | 993.6 | 99.8 |
| 174 | 1036.29 | 1035.6 | 99.0 |
| 175 | 1030.26 | 1029.4 | 99.0 |
| 176 | 1115.37 | 1114.6 | 95.5 |
| 177 | 1045.31 | 1045.2 | 99.8 |
| 178 | 1087.32 | 1086.6 | 97.8 |
| 179 | 847.03 | 846.8 | 97.5 |
| 180 | 862.05 | 862.2 | 93.7 |
| 181 | 880.04 | 879.9 | 99.1 |
| 182 | 896.50 | 896.3 | 96.2 |
| 183 | 904.09 | 904.4 | 98.0 |
| 184 | 922.08 | 922.3 | 98.7 |
| 185 | 938.54 | 938.1 | 98.9 |
| 186 | 1018.24 | 1017.7 | 92.3 |
| 187 | 1036.23 | 1036.4 | 93.9 |
| 188 | 1052.69 | 1052.5 | 98.4 |
| 189 | 1060.28 | 1060.4 | 97.3 |
| 190 | 1078.27 | 1078.6 | 98.3 |
| 191 | 1094.72 | 1094.3 | 99.5 |
| 192 | 1352.6 | 1352.48 | 90.0 |
| 193 | 918.1 | | 90.0 |
| 194 | 1132.3 | | 90.0 |
| 195 | 1074.3 | 1073.7 | 99.0 |
| 196 | 1237.5 | | 99.0 |
| 197 | 1366.6 | | 78.0 |
| 198 | 904.09 | 903.5 | 84.7 |

### EXAMPLE 188

### Construction of MC Receptor Expression Plasmids

Construction of human MC1 expression plasmid: Human MC1 cDNA is cloned by PCR using human genomic DNA (Clontech Cat. # 6550-1) as a template. A forward hMC1 gene-specific primer containing initiation codon (ATG) and EcoRI site and a reverse hMC1 gene specific primer containing a stop codon and XbaI site are used in the PCR. The full-length hMC1 cDNA generated by PCR is cloned into pUC18/SmaI plasmid (Pharmacia Cat. # 27-5266-01), and the correct hMC1 cDNA is confirmed by DNA sequencing. The sequenced pUC18hMC1 is digested with EcoRI and XbaI, and the hMC1 cDNA fragment is then subcloned into pcDNA3.1 (Invitrogen Cat. # V790-20) to generate expression plasmid pCDNA3-hMC1.

Construction of human MC3 expression plasmid: Human MC3 cDNA is cloned by PCR using human genomic DNA (Clontech Cat. # 6550-1) as a template. A forward hMC3 gene-specific primer containing initiation codon (ATG) and EcoRI site and a reverse hMC3 gene specific primer containing a stop codon and XbaI site are used in the PCR. The full-length hMC3 cDNA generated by PCR is cloned into pUC18/SmaI plasmid (Pharmacia Cat# 27-5266-01), and the correct hMC3 cDNA is confirmed by DNA sequencing. The sequenced pUC18hMC3 is digested with EcoRI and XbaI, and the hMC3 cDNA fragment is then subcloned into pcDNA3.1 (Invitrogen Cat. # V790-20) to generate expression plasmid pCDNA3-hMC3.

Construction of human MC4 expression plasmid: Human MC4 (hMC4) cDNA is cloned in a similar way as hMC3 cDNA by PCR using human fetal brain cDNA-(Clontech Cat. # 7402-1) as a template. The hMC4 cDNA PCR product is digested with EcoRI/XbaI, and then subcloned into pCIneo (Promega Cat. # E1841) and sequenced. The resulting hMC4R plasmid has two mutations, which are then corrected to create the hMC4 cDNA encoding the correct hMC4 protein. The corrected hMC4 cDNA is then subcloned into pcDNA3.1 to generate expression plasmid pCDNA3-hMC4.

Construction of human MC5 expression plasmid: Human MC5 cDNA is cloned by PCR using human genomic DNA (Clontech Cat. # 6550-1) as a template. A forward hMC5 gene-specific primer containing initiation codon (ATG) and HindIII site and a reverse hMC5 gene specific primer containing a stop codon and XbaI site are used in the PCR. The full-length hMC5 cDNA generated by PCR is cloned into pUC18/SmaI plasmid (Pharmacia Cat. # 27-5266-01), and the correct hMC5 cDNA is confirmed by DNA sequencing. The sequenced pUC18hMC5 is digested with EcoRI and XbaI, and the hMC5 cDNA fragment is then subcloned into pcDNA3.1 (Invitrogen Cat. # V790-20) to generate expression plasmid pCDNA3-hMC5.

Stable HEK-293 cells expressing human MCRs: Stable 293 cells expressing all hMCRs are generated by co-transfecting HEK-293 cells with pCDNA3-hMC4R and a CRE-luciferase reporter plasmid following the protocol of Lipofectamine Plus Reagent (Invitrogen, Cat. # 10964-013). For selection of stable transfectants, the Genticin (G418) is added to the media at a concentration of 300 μg/mL 48 hours after the start of transfection. After 2-3 weeks, 40-50 of isolated clones are selected, propagated, and assayed for luciferase activity using a Luciferase Reporter Gene Assay kit (Roche, Cat. # 1814036). Around five stable clones with highly stimulated luciferase activities by 10 nM NDP-αMSH are established.

### EXAMPLE 189

### Melanocortin Receptor Whole Cell cAMP Accumulation Assay

Hank's Balanced Salt Solution without phenol red (HBSS-092), 1 M HEPES, Dulbecco's Modified Eagle Media (DMEM), Fetal Bovine Serum (FBS), Antibiotic/Antimycotic Solution, and sodium acetate are obtained from GibcoBRL. Triton X-100, ascorbic acid, cAMP, and 3-isobutyl-1-methyl-xanthine (IBMX) are purchased from Sigma. Bovine Serum Albumin (BSA) is obtained from Roche. SPA PVT antibody-binding beads type II anti-sheep beads and ¹²⁵I cAMP are obtained from Amersham. Anti-goat cAMP antibody is obtained from ICN. Enzyme Free Cell Dissociation Solution Hank's based is obtained from Specialty Media. NDP-αMSH is obtained from Calbiochem. Dimethylsulfoxide (DMSO) is obtained from Aldrich.

Compound Preparation

In the agonist assay, compounds are prepared as 10 mM and NDP-AMSH (control) as 33.3 μM stock solutions in 100% DMSO. These solutions are serially diluted in 100% DMSO. The compound plate is further diluted in compound dilution buffer (HBSS-092, 1 mM Ascorbic Acid, 1 mM IBMX, 0.6% DMSO, 0.1% BSA) to yield a final concentration range in the assay between 600 nM-6 pM for compound and 100 nM-1 pM for NDP-αMSH control in 0.5% DMSO. Twenty μL of compound solution are transferred from this plate into four PET 96-well plates (all assays are performed in duplicate for each receptor).

Cell Culture and Cell Stimulation

HEK 293 cells stably transfected with the human MC3R or MC4R are grown in DMEM containing 10% FBS and 1% Antibiotic/Antimycotic Solution. On the day of the assay, the cells are dislodged with enzyme free cell dissociation solution and re-suspended in cell buffer (HBSS-092, 0.1% BSA, 10 mM HEPES) at 1×10⁶ cells/mL. Forty μL of cell suspension are added per well to PET 96-well plates containing 20 μL of diluted compound or control. Plates are incubated at 37° C. in a waterbath for 20 minutes. The assay is stopped by adding 50 μL Quench Buffer (50 mM sodium acetate, 0.25% Triton X-100).

Determination of cAMP Concentrations

Radioligand binding assays are run in SPA buffer (50 mM sodium acetate, 0.1% BSA). The beads, antibody, and radioligand are diluted in SPA buffer to provide sufficient volume for each 96-well plate. To each quenched assay well is added 100 μL cocktail containing 33.33 μL of beads, 33.33 μL antibody, and 33.33 μL ¹²⁵I-cAMP. This is based on a final concentration of 6.3 mg/mL beads, 0.65% anti-goat antibody, and 61 pM of ¹²¹I-cAMP (containing 25,000-30,000 CPM) in a final assay volume of 210 μL. The plates are counted in a Wallac MicroBeta counter after a 12-hour incubation.

The data are converted to pmol of cAMP using a standard curve assayed under the same conditions. The data are analyzed using Activity Base software to generate agonist potencies (EC50), and percent relative efficacy data compared to NDP-αMSH.

TABLE 3
MC4 Potency and Selectivity

| | | MC1/MC4 |
|---|---|---|
| Compound No. | MC4 Kᵢ (nM) | selectivity |
| 1 | 127.80 | 3.91 |
| 2 | 0.39 | 10.70 |
| 3 | 0.41 | 4.00 |
| 4 | 0.23 | 0.26 |
| 5 | 0.42 | 5.00 |
| 6 | 2.15 | 35.74 |
| 7 | 0.82 | 15.00 |
| 8 | 1.43 | 3.33 |
| 9 | 2.39 | 10.00 |
| 10 | 0.10 | 9.50 |
| 11 | 1.26 | 11.00 |
| 12 | 1.10 | 6.72 |
| 13 | 0.34 | 10.65 |
| 14 | 0.35 | 12.54 |
| 15 | 0.67 | 14.75 |
| 16 | 0.83 | 2.94 |
| 17 | 0.57 | 10.42 |
| 18 | 0.35 | 8.15 |
| 19 | 0.53 | 7.64 |
| 20 | 0.48 | 4.81 |
| 21 | 0.22 | 10.27 |
| 22 | 0.27 | 6.85 |
| 23 | 0.26 | 10.54 |
| 24 | 0.44 | 8.00 |
| 25 | 0.32 | 11.00 |
| 26 | 0.71 | 38.90 |
| 27 | 1.05 | 30.11 |
| 28 | 1.18 | 26.35 |
| 29 | 3.18 | 15.00 |
| 30 | 2.36 | 38.48 |
| 31 | 0.75 | 57.02 |
| 32 | 0.37 | 66.88 |
| 33 | 0.35 | 79.54 |
| 34 | 43.42 | 11.52 |
| 35 | 1.03 | 1.17 |
| 36 | 1.66 | 1.22 |
| 37 | 1.81 | 36.99 |
| 38 | 2.55 | 28.16 |
| 39 | 2.08 | 19.67 |
| 40 | 0.96 | 25.92 |
| 41 | 0.60 | 58.47 |
| 42 | 0.40 | 44.63 |
| 43 | 1.06 | 11.00 |
| 44 | 0.95 | 15.00 |
| 45 | 3.03 | 30.47 |
| 46 | 0.73 | |
| 47 | 53.32 | |
| 48 | 0.43 | 26.80 |
| 49 | 3.14 | 35.35 |
| 50 | 0.21 | 36.10 |
| 51 | 6.52 | 76.75 |
| 52 | 0.55 | 30.54 |
| 53 | 8.68 | |
| 54 | 0.48 | 20.85 |
| 55 | 1.67 | 28.81 |
| 56 | 23.39 | 21.38 |
| 57 | 2.26 | 29.00 |
| 58 | 0.81 | 31.69 |
| 59 | 0.86 | 20.92 |
| 60 | 1.51 | 29.95 |
| 61 | 0.87 | 1.70 |
| 62 | 0.75 | 46.91 |
| 63 | 2.28 | 30.51 |
| 64 | 0.62 | 4.12 |
| 65 | 6.53 | 2.70 |
| 66 | 0.83 | 13.23 |
| 67 | 0.26 | 9.15 |
| 68 | 0.63 | 14.08 |
| 69 | 3.00 | 18.38 |
| 70 | 0.30 | 2.00 |
| 71 | 2.11 | 5.13 |
| 72 | 0.78 | 22.31 |
| 73 | 8.78 | 12.77 |
| 74 | 1.21 | 12.00 |
| 75 | 2.31 | 6.00 |
| 76 | 24.23 | 6.00 |
| 77 | 0.41 | 28.38 |
| 78 | 7.28 | 9.00 |
| 79 | 0.57 | 21.79 |
| 80 | 5.27 | 8.24 |
| 81 | 5.93 | 101.69 |
| 82 | 300.86 | 1.66 |
| 83 | 0.26 | 45.95 |
| 84 | 3.32 | 150.60 |
| 85 | 188.06 | 2.66 |
| 86 | 0.13 | 66.21 |
| 87 | 1.11 | 316.25 |
| 88 | 55.14 | 9.07 |
| 89 | 0.11 | 71.43 |
| 90 | 0.86 | 237.22 |
| 91 | 23.65 | 21.14 |
| 92 | 0.52 | 12.06 |
| 93 | 0.65 | 1.48 |
| 94 | 5.12 | 16.97 |
| 95 | 155.83 | 3.21 |
| 96 | 4.01 | 20.87 |
| 97 | 0.58 | 8.03 |
| 98 | 11.54 | 7.43 |
| 99 | 5.66 | 88.42 |
| 100 | 300.24 | 1.67 |
| 101 | 14.00 | 0.97 |
| 102 | 105.01 | 4.76 |
| 103 | 6.62 | 75.59 |
| 104 | 135.91 | 3.68 |
| 105 | 20.80 | 24.04 |
| 106 | 20.88 | 23.95 |
| 107 | 500.00 | 1.00 |
| 108 | 31.36 | 5.99 |
| 109 | 82.70 | 6.05 |
| 110 | 117.22 | 4.27 |
| 111 | 65.19 | 7.67 |
| 112 | 88.97 | 5.62 |
| 113 | 37.01 | 13.51 |
| 114 | 1.35 | 4.00 |
| 115 | 1.15 | 2.00 |
| 116 | 2.00 | 4.00 |
| 117 | 0.63 | 1.00 |
| 118 | 4.59 | 4.52 |
| 119 | 0.57 | 0.86 |
| 120 | 0.40 | 1.00 |
| 121 | 0.34 | 0.74 |
| 122 | 0.30 | 0.90 |
| 123 | 1.13 | 2.42 |
| 124 | 2.36 | 18.11 |
| 125 | 19.94 | 25.08 |
| 126 | 0.74 | 22.64 |
| 127 | 0.28 | 20.25 |
| 128 | 0.89 | 22.46 |
| 129 | 2.18 | 22.16 |
| 130 | 1.98 | 26.88 |
| 131 | 11.18 | 7.00 |
| 132 | 0.34 | 77.32 |
| 133 | 9.08 | 31.29 |
| 134 | 0.13 | 68.42 |
| 135 | 0.06 | 120.27 |
| 136 | 55.30 | 7.01 |
| 137 | 0.32 | 54.60 |
| 138 | 3.08 | 38.81 |
| 139 | 0.38 | 44.29 |
| 140 | 0.20 | 128.15 |
| 141 | 0.19 | 83.00 |
| 142 | 0.11 | 35.55 |
| 143 | 0.08 | 19.27 |
| 144 | 0.30 | 14.85 |
| 145 | 0.73 | 3.82 |
| 146 | 0.32 | 27.43 |
| 147 | 0.07 | 0.86 |
| 148 | 0.10 | 51.98 |
| 149 | 0.07 | 51.85 |
| 150 | 2.35 | 12.88 |
| 151 | 4.35 | 14.00 |
| 152 | 1.77 | 7.73 |
| 153 | 0.10 | 41.81 |
| 154 | 0.21 | 36.00 |
| 155 | 0.68 | 55.84 |
| 156 | 1.31 | 158.41 |
| 157 | 28.42 | 17.60 |
| 158 | 0.08 | 50.25 |
| 159 | 0.74 | 49.41 |
| 160 | 0.05 | 0.90 |
| 161 | 0.08 | 2.18 |
| 162 | 0.08 | 30.07 |
| 163 | 2.28 | 19.46 |
| 164 | 0.38 | 7.79 |
| 165 | 1.45 | 13.53 |
| 166 | 25.05 | 9.38 |
| 167 | 93.07 | 3.36 |
| 168 | 1.35 | 212.71 |
| 169 | 0.03 | 1804.00 |
| 170 | 0.13 | 9.00 |
| 171 | 0.10 | 75.11 |
| 172 | 0.15 | 26.45 |
| 173 | 0.37 | 29.10 |
| 174 | 0.23 | 4.98 |
| 175 | 1.29 | |
| 176 | 0.49 | |
| 177 | 0.05 | |
| 178 | 0.38 | |
| 179 | 93.46 | 5.35 |
| 180 | 16.46 | 30.38 |
| 181 | 6.07 | 45.25 |
| 182 | 0.89 | 185.74 |
| 183 | 9.37 | 53.39 |
| 184 | 2.51 | 97.44 |
| 185 | 0.47 | 269.59 |
| 186 | 5.21 | 11.44 |
| 187 | 2.02 | 20.76 |
| 188 | 0.92 | 29.56 |
| 189 | 2.72 | 23.31 |
| 190 | 0.17 | 367.10 |
| 191 | 0.26 | 127.33 |
| 192 | 36.70 | 1.00 |
| 193 | 2.59 | 26.59 |
| 194 | 2.93 | 10.61 |
| 195 | 0.87 | 32.56 |
| 196 | 2.10 | 4.98 |
| 197 | 21.81 | 1.00 |
| 198 | 16.72 | 13.07 |
| 191 | 0.26 | 127.33 |
| 192 | 36.70 | 1.00 |
| 193 | 2.59 | 26.59 |
| 194 | 2.93 | 10.61 |
| 195 | 0.87 | 32.56 |
| 196 | 2.10 | 4.98 |
| 197 | 21.81 | 1.00 |
| 198 | 16.72 | 13.07 |

1-10. (canceled)

11. A compound selected from the group consisting of Compound Numbers 1-198.

12. (canceled)

13. The compound of claim 11, wherein the compound is AC-D-Arg-cyclo[Cys-Glu-His-D-Phe-Arg-Trp-Cys]-NH₂.

14. The compound of claim 11, wherein the compound is Ac-cyclo[hCys-His-D-Phe-Arg-Trp-Cys]-NH₂.

15. A pharmaceutical composition comprising a pharmaceutically acceptable carrier and at least one compound as claimed by claim 11.

16. A method for agonizing the MC4 receptor, comprising the step of administering to a patient in need thereof a pharmaceutically effective amount of at least one compound of any one as claimed in claim 11.

17. A method of treating obesity in a mammal, comprising the step of administering to the mammal in need thereof a pharmaceutically effective amount of at least one compound of any one as claimed in claim 11.

18. A method of treating diabetes mellitus in a mammal, comprising the step of administering to the mammal in need thereof a pharmaceutically effective amount of at least one compound as claimed in claim 11.

19. A method of treating male and/or female sexual dysfunction in a mammal, comprising the step of administering to the mammal in need thereof a pharmaceutically effective amount of at least one compound as claimed in claim 11.

20-23. (canceled)

24. The compound of claim 11, wherein the compound is Ac-Arg-cyclo[Cys-Glu-His-D-Phe-Arg-Trp-Cys]-NH₂.

25. The compound of claim 11, wherein the compound is cyclo[hCys-His-D-Phe-Arg-Trp-Cys]-NH₂.

26. The compound of claim 11, wherein the compound is 3-guanidinopropionyl-cyclo[hCys-His-D-Phe-Arg-Trp-Cys]-NH₂.

27. The compound of claim 11, wherein the compound is 5-guanidinovaleryl-cyclo[hCys-His-D-Phe-Arg-Trp-Cys]-NH₂.

28. The compound of claim 11, wherein the compound is Arg-cyclo[hCys-His-D-Phe-Arg-Trp-Cys]-OH.
